(19) United States
(12) Patent Application Publication
(10) Pub. No.: US 2026/0308656 A1
(43) Pub. Date: Oct. 8, 2026

(54) METHODS AND DEVICES FOR THE RAPID SEPARATION AND DETECTION OF MICROBIAL CELLS

(71) Applicant: SHEMRON HOLDINGS INC., NORTH YORK (CA)

(72) Inventors: SAMAD TALEBPOUR, RICHMOND HILL (CA); STEPHEN WESLEY LEONARD, MARKHAM (CA); ANNA KHIMCHENKO, THORNHILL (CA); SUKHDEV MANKU, TORONTO (CA)

(21) Appl. No.: 19/479,593

(22) PCT Filed: Apr. 26, 2024
(86) PCT No.: PCT/CA2024/050572

§ 371 Date: Oct. 28, 2025

Related U.S. Application Data
(60) Provisional application No. 63/462,857, filed on Apr. 28, 2023.
(60) Provisional application No. 63/472,222, filed on Jun. 9, 2023.

(51) Int. Cl. C12Q 1/04; C12Q 1/24; C12Q 1/6813; C12Q 1/6844; C12Q 1/6869; C12Q 1/689

(52) U.S. Cl. CPC C12Q 1/04; C12Q 1/24; C12Q 1/6813; C12Q 1/6844; C12Q 1/6869; C12Q 1/689

(57) ABSTRACT

Methods are provided for the rapid detection of microbial cells. In some embodiments, microbial cells are obtained from a sample and inoculated onto a solid growth medium, incubated and monitored to detect microcolonies. Microbial cells from a microcolony ate harvested to provide microbial cells for rapid nucleic acid amplification and/or sequencing. The microbial cells may be obtained from a sample containing whole blood by lysing host blood cells with a blood lysis reagent and separating the microbial cells to obtain a suspension. A method of separating microbial cells from a whole blood sample is provided in which the whole blood sample is mixed with a blood lysis reagent containing saponin and sodium polyanethole sulfonate to form a first mixture, and subsequently mixed with an alkaline buffer to form a second mixture. A separation process is then employed to separate microbial cells from the second mixture.

### CROSS-REFERENCE TO RELATED APPLICATION

This application claims priority to U.S. Provisional Patent Application No. 63/462,857, titled “METHODS FOR RAPIDLY DETECTING ANTIMICROBIAL RESISTANCE FROM HARVESTED MICROCOLONIES” and filed on Apr. 28, 2023, the entire contents of which is incorporated herein by reference, and claims priority to U.S. Provisional Patent Application No. 63/472,222, titled “METHODS AND DEVICES FOR THE RAPID SEPARATION AND DETECTION OF MICROBIAL CELLS” and filed on Jun. 9, 2023, the entire contents of which is incorporated herein by reference, and claims priority to U.S. Provisional Patent Application No. 63/537,732, titled “METHODS AND DEVICES FOR THE RAPID SEPARATION AND DETECTION OF MICROBIAL CELLS” and filed on Sep. 11, 2023, the entire contents of which is incorporated herein by reference.

### BACKGROUND

The present disclosure relates to the growth, detection and characterization of microbial cells.

Identifying causative organisms of microbial infection and determining their antimicrobial susceptibility profile is the main goal of diagnostic routing in clinical microbiology laboratories. As a common practice, this task is currently performed by drawing patient blood into culture bottles containing antibiotic absorbing agents, incubating the bottle in an environment that promotes growth of the blood microbial cell content, performing Gram stain to classify bacterial cells in terms of cell wall characteristic and morphology, sub-culturing the cells on solid growth media such as agar plates to obtain pure microbial colonies, partially or fully identifying the microbial cells, suspending the colony content in a media in a manner by which the cell concentration falls in a desired range, incubating aliquots of the cell-suspension in contact with different doses of selected antimicrobials in appropriate medium, and determining the minimum inhibitory concentration (MIC) from the growth profiles of the cell aliquots. The major shortcomings of this diagnostic routing are the long time to result (of order of few days) and the possibility of preferential growth in the case of polymicrobial samples.

### SUMMARY

Methods are provided for the rapid detection of microbial cells. In some embodiments, microbial cells are obtained from a sample and inoculated onto a solid growth medium, incubated and monitored to detect microcolonies. Microbial cells from a microcolony may be harvested to provide microbial cells for subsequent analysis, such as rapid nucleic acid amplification and/or sequencing. The microbial cells may be obtained from a sample containing whole blood by lysing host blood cells with a blood lysis reagent and separating the microbial cells to obtain a suspension. A method of separating microbial cells from a whole blood sample is provided in which the whole blood sample is mixed with a blood lysis reagent containing saponin and sodium polyanethole sulfonate to form a first mixture, and subsequently mixed with an alkaline buffer to form a second mixture. A separation process is then employed to separate microbial cells from the second mixture.

Accordingly, in a first aspect, there is provided a method of performing nucleic acid amplification on nucleic acids derived from a microcolony of microbial cells, the method comprising:
detecting a presence of the microcolony on a solid growth medium;
harvesting microbial cells from the microcolony while a diameter of the microcolony remains below 100 microns, thereby obtaining harvested microbial cells;
lysing the harvested microbial cells, thereby obtaining a lysate; and
in the absence of an intervening growth step, performing nucleic acid amplification to amplify at least one nucleic acid residing within the lysate, thereby obtaining an amplification product.

In some example implementations, the method further comprises detecting a presence of the amplification product.

In some example implementations of the method, at least one nucleic acid amplified by nucleic acid amplification comprises an antimicrobial resistance gene.

In some example implementations of the method, the nucleic acid amplification is configured to amplify a plurality of nucleic acids.

In some example implementations of the method, at least one of the plurality of nucleic acids comprises an antimicrobial resistance gene.

In some example implementations of the method, the plurality of nucleic acids comprise a set of antimicrobial resistance genes.

In some example implementations of the method, at least one of the plurality of nucleic acids comprises an antimicrobial resistance gene and at least one of the plurality of nucleic acids is uniquely associated with a microbial species.

In some example implementations of the method, at least one of the plurality of nucleic acids comprises an antimicrobial resistance gene and at least one of the plurality of nucleic acids is uniquely associated with a microbial genus.

In some example implementations of the method, at least one of the plurality of nucleic acids comprises an antimicrobial resistance gene and at least one of the plurality of nucleic acids is uniquely associated with a microbial strain.

In some example implementations of the method, at least two of the plurality of nucleic acids are respectively associated with antimicrobial resistance genes, and wherein at least two of the plurality of plurality of nucleic acids are respectively associated with a unique microbial genus, strain or species.

In some example implementations of the method, at least two of the nucleic acids are amplified in a multiplexed amplification reaction.

In some example implementations of the method, the nucleic acid amplification is performed in absence of performing nucleic acid extraction from the lysate.

In some example implementations of the method, the microbial cells are Gram positive microbial cells and lysing of the microbial cells is performed by heat lysis.

In some example implementations, the method further comprises performing sequencing on the amplification product. The nucleic acid amplification step may comprise whole genome amplification.

In some example implementations, the method further comprises, prior to detecting the presence of the microcolony:
obtaining a sample;
in the absence of culturing the sample, inoculating microbial cells from the sample onto the solid growth medium; and
incubating the solid growth medium and monitoring the solid growth medium for the presence of one or more microcolonies.

Inoculating microbial cells from the sample onto the solid growth medium may comprise directly contacting the sample with the solid growth medium.

In some example implementations of the method, the sample is a urine sample.

In some example implementations, the method further comprises, prior to inoculating microbial cells from the sample onto the solid growth medium:
separating microbial cells from the sample and resuspending the microbial cells to obtain a microbial suspension;
wherein inoculating the microbial cells from the sample comprises contacting the microbial suspension with the solid growth medium.

In some example implementations of the method, the sample comprises whole blood, and wherein separating the microbial cells from the sample comprises:
contacting the sample with an alkaline blood lysis reagent comprising saponin and sodium polyanethole sulfonate, thereby obtaining a mixture;
separating microbial cells from the mixture and resuspending the microbial cells to obtain the microbial suspension.

In some example implementations of the method, the microbial cells are inoculated onto the solid growth medium such that an areal fraction of residual sample debris is between 10% and 50%.

In some example implementations of the method, the microbial cells are inoculated onto the solid growth medium such that an areal fraction of residual sample debris is between 10% and 40%.

In some example implementations of the method, the microbial cells are inoculated onto the solid growth medium such that an areal fraction of residual sample debris is between 10% and 30%.

In some example implementations of the method, the microbial cells are inoculated onto the solid growth medium such that an average lateral dimension of residual sample debris is between 1 and 10 micrometers.

In some example implementations of the method, the microcolony is a first microcolony, and wherein a second microcolony is detected on the solid growth medium, the method further comprising:
after harvesting the first microcolony, continuing to monitor the second microcolony until the second microcolony grows to a size suitable for performing a downstream assay;
harvesting the second microcolony; and
employing microbial cells harvested from the second microcolony to perform the downstream assay.

In some example implementations of the method, the downstream assay is selected from matrix-assisted laser desorption/ionization and antimicrobial susceptibility testing.

In another aspect, there is provided a method of performing nucleic acid amplification on nucleic acids derived from a microcolony of microbial cells, the method comprising:
detecting a presence of a microcolony on a solid growth medium;
monitoring the microcolony to determine when the microcolony has grown to a colony that contains a sufficient biomass to facilitate sequencing;
harvesting microbial cells from the colony;
lysing the harvested microbial cells, thereby obtaining a lysate; and
in the absence of an intervening growth step, and in the absence of an intervening amplification step, employing nucleic acids from the lysate to perform library preparation for sequencing.

In some example implementations, the method further comprises performing sequencing based on the prepared library.

In another aspect, there is provided a method of performing a Gram stain microbial cells harvested from a microcolony, the method comprising:
detecting a presence of the microcolony on a solid growth medium;
harvesting microbial cells from the microcolony while a diameter of the microcolony remains below 100 microns, thereby obtaining harvested microbial cells;
in the absence of an intervening growth step, performing a Gram stain on the harvested microbial cells.

In another aspect, there is provided a method of performing nucleic acid amplification on nucleic acids derived from a microcolony of microbial cells, the method comprising:
detecting a presence of the microcolony on a solid growth medium;
harvesting material from the microcolony while a diameter of the microcolony remains below 100 microns; and
in the absence of an intervening growth step, performing nucleic acid amplification on nucleic acids residing with the harvested material.

In another aspect, there is provided a method of detecting an infection, the method comprising:
obtaining a sample;
in the absence of culturing the sample, inoculating microbial cells from the sample onto a solid growth medium;
incubating the solid growth medium and monitoring the solid growth medium for a presence of one or more microcolonies;
detecting a plurality of microcolonies on the solid growth medium; and
employing the number of microcolonies to determine a measure associated with a severity of the infection.

In another aspect, there is provided a method of monitoring an infection, the method comprising:
a) obtaining a sample;
b) in the absence of culturing the sample, inoculating microbial cells from the sample onto a solid growth medium; and
c) incubating the solid growth medium and monitoring the solid growth medium for a presence of one or more microcolonies.
d) detecting a plurality of microcolonies on the solid growth medium;
e) repeating steps a) to d) after a time delay;
f) repeating step e) one or more times; and
g) employing a time dependence of the number of detected microcolonies to monitor an infection.

In another aspect, there is provided method of processing a whole blood sample suspected of containing microbial cells, the method comprising:
collecting the whole blood sample and mixing the whole blood sample with a blood lysis reagent to obtain a first mixture, the blood lysis reagent comprising saponin and sodium polyanethole sulfonate;
contacting the first mixture with an alkaline buffer and obtaining a second mixture with a viscosity that is less than a viscosity of the first mixture; and
separating microbial cells from the second mixture to obtain a suspension comprising the microbial cells.

In some example implementations of the method, the first mixture is contacted with the second mixture after a time delay between 15 minutes and 24 hours to permit partial digestion of blood debris within the first mixture prior to contact with the alkaline buffer.

In some example implementations of the method, the first mixture is contacted with the second mixture after a time delay between 30 minutes and 24 hours to permit partial digestion of blood debris within the first mixture prior to contact with the alkaline buffer.

In some example implementations of the method, the first mixture is contacted with the second mixture after a time delay between 40 minutes and 24 hours to permit partial digestion of blood debris within the first mixture prior to contact with the alkaline buffer.

In some example implementations of the method, the first mixture is contacted with the second mixture after a time delay between 15 minutes and 1 hour to permit partial digestion of blood debris within the first mixture prior to contact with the alkaline buffer.

In some example implementations of the method, the first mixture is contacted with the second mixture after a time delay between 30 minutes and 1 hour to permit partial digestion of blood debris within the first mixture prior to contact with the alkaline buffer.

In some example implementations of the method, the first mixture is contacted with the second mixture after a time delay between 40 minutes and 1 hour to permit partial digestion of blood debris within the first mixture prior to contact with the alkaline buffer.

In some example implementations of the method, a volume of the whole blood sample is between 5 ml and 10 ml.

In some example implementations, the method further comprises lysing microbial cells within the suspension to obtain a lysate; and performing nucleic acid amplification to amplify at least one nucleic acid residing within the lysate, thereby obtaining an amplification product. The method may further comprise detecting a presence of the amplification product.

In some example implementations of the method, at least one nucleic acid amplified by nucleic acid amplification comprises an antimicrobial resistance gene.

In some example implementations of the method, the nucleic acid amplification is configured to amplify a plurality of nucleic acids. At least one of the plurality of nucleic acids may comprise an antimicrobial resistance gene. The plurality of nucleic acids may comprise a set of antimicrobial resistance genes. At least one of the plurality of nucleic acids may comprise an antimicrobial resistance gene and at least one of the plurality of nucleic acids is uniquely associated with a microbial species. At least one of the plurality of nucleic acids may comprise an antimicrobial resistance gene and at least one of the plurality of nucleic acids is uniquely associated with a microbial genus. At least one of the plurality of nucleic acids may comprise an antimicrobial resistance gene and at least one of the plurality of nucleic acids is uniquely associated with a microbial strain. At least two of the plurality of nucleic acids may be respectively associated with antimicrobial resistance genes, and wherein at least two of the plurality of plurality of nucleic acids are respectively associated with a unique microbial genus, strain or species. At least two of the nucleic acids may be amplified in a multiplexed amplification reaction.

In some example implementations of the method, the nucleic acid amplification is performed in absence of performing nucleic acid extraction from the lysate.

In some example implementations, the method further comprises performing sequencing on the amplification product.

In some example implementations, the method further comprises inoculating the suspension of microbial cells onto a solid growth medium; and monitoring the solid growth medium for a presence of a microcolony. The method may further comprise detecting the presence of the microcolony.

In some example implementations of the method, a composition of the blood lysis reagent and the time delay between forming the first mixture and contacting the first mixture with the alkaline buffer may be selected such that after inoculating the suspension of microbial cells onto the solid growth medium, residual blood debris from the suspension partially covers an inoculated surface of the solid growth medium with a spatial areal fraction of less than 50 percent.

In some example implementations of the method, a composition of the blood lysis reagent and the time delay between forming the first mixture and contacting the first mixture with the alkaline buffer are selected such that after inoculating the suspension of microbial cells onto the solid growth medium, residual blood debris from the suspension partially covers an inoculated surface of the solid growth medium with a spatial areal fraction of less than 40 percent.

In some example implementations of the method, a composition of the blood lysis reagent and the time delay between forming the first mixture and contacting the first mixture with the alkaline buffer are selected such that after inoculating the suspension of microbial cells onto the solid growth medium, residual blood debris from the suspension partially covers an inoculated surface of the solid growth medium with a spatial areal fraction of less than 30 percent.

In some example implementations of the method, a composition of the blood lysis reagent and the time delay between forming the first mixture and contacting the first mixture with the alkaline buffer are selected such that after inoculating the suspension of microbial cells onto the solid growth medium, residual blood debris from the suspension partially covers an inoculated surface of the solid growth medium with a spatial areal fraction of less than 20 percent.

In some example implementations of the method, a composition of the blood lysis reagent and the time delay between forming the first mixture and contacting the first mixture with the alkaline buffer are selected such that a mean size of residual blood debris particles residing within the suspension is less than 10 micrometers.

In some example implementations of the method, the whole blood sample is collected into an evacuated vessel containing the blood lysis reagent.

In some example implementations of the method, the microbial cells are separated from the second mixture within one hour of contacting the first mixture with the alkaline buffer.

In some example implementations of the method, the blood lysis reagent is provided such that, after mixing the blood lysis reagent with the whole blood sample, the first mixture comprises a concentration of saponin between 0.75 and 60 mg/ml and concentration of sodium polyanethole sulfonate between 0.35 and 50 mg/ml.

In some example implementations of the method, a pH of the blood lysis reagent, prior to contact with the whole blood sample, resides between 3.5 and 8.

In some example implementations of the method, a concentration of calcium ions in the blood lysis reagent, relative to dry weight of saponin, is less than 0.5% w/w.

In some example implementations of the method, a concentration of calcium ions in the blood lysis reagent, relative to dry weight of saponin, is between 0.1% w/w and 0.5% w/w.

In some example implementations of the method, a pH of the alkaline buffer is between 8 to 9.

In some example implementations of the method, the blood lysis reagent further comprises cyclodextrin having a concentration between 0.1 mM and 20 mM.

In some example implementations of the method, the blood lysis reagent further comprises cyclodextrin having a concentration between 1 to 5 mM per 3% w/w of saponin.

In some example implementations of the method, the whole blood sample is collected and mixed with the blood lysis reagent at a collection site, and wherein the first mixture is received at a processing location that is remote from the collection site prior to contacting the first mixture with the alkaline buffer.

In some example implementations, the method further comprises transporting the first mixture from the collection site to the processing location.

In some example implementations of the method, the alkaline buffer is mixed with the first mixture within a fluidic cartridge, and an automated instrument is employed to process the fluidic cartridge to performed automated separation of the microbial cells.

In some example implementations of the method, the second mixture is formed by contacting a portion of the first mixture with the alkaline buffer.

In another aspect, there is provided a kit comprising for processing a whole blood sample suspected of containing microbial cells, the kit comprising:
a vessel comprising a blood lysis reagent, the blood lysis reagent comprising saponin and sodium polyanethole sulfonate, the vessel having a sufficiently low internal pressure to facilitate collection of the whole blood sample between 5 ml and 10 ml from a subject, such that when the whole blood sample is drawn into the vessel under pressure, a first mixture is formed; and
a second vessel comprising an alkaline buffer, the alkaline buffer being configured such that after mixing the first mixture with the alkaline buffer to form a second mixture, a viscosity of the second mixture is less than a viscosity of the first mixture.

In some example implementations of the kit, a concentration of calcium ions in the blood lysis reagent is less than 0.5% w/w.

In some example implementations of the kit, a concentration of calcium ions in the blood lysis reagent is between 0.1% w/w and 0.5% w/w.

In some example implementations of the kit, the blood lysis reagent further comprises cyclodextrin having a concentration between 0.1 mM and 20 mM.

In another aspect, there is provided a lytic blood collection vessel comprising a blood lysis reagent, the blood lysis reagent comprising saponin and sodium polyanethole sulfonate, the vessel having a pressure sufficiently to facilitate collection of a whole blood sample of between 5 ml and 10 ml from a subject, such that when the whole blood sample is drawn into the vessel, a first mixture is formed, wherein a concentration of calcium ions in the blood lysis reagent is between 0.1% and 0.5% w/w.

In another aspect, there is provided a method of harvesting a microcolony from a solid growth medium, the method comprising:
applying a potential difference between the solid growth medium and an electrically conductive elongate member, such that the electrically conductive elongate member has a positive polarity relative to the solid growth medium; and
bringing a distal end region of the electrically conductive elongate member into sufficiently close proximity with the microcolony to facilitate electric-field-mediated collection of microbial cells from the microcolony onto the distal end region.

In some example implementations of the method, the distal end region comprises an electrically insulating layer preventing direct electrical contact between an electrically conductive portion of the electrically conductive elongate member and the collected microbial cells.

In some example implementations of the method, the potential difference is between 3V and 50V.

In another aspect, there is provided a method of harvesting a microcolony from a solid growth medium, the method comprising:
providing a fluidic transfer device comprising a fluidic transfer tube, the fluidic transfer tube having a distal end comprising an aperture suitable for aspirating and dispensing fluids;
controlling the fluidic transfer device to aspirate a harvesting buffer;
positioning the fluidic transfer tube such that a distal end thereof contacts the solid growth medium with the aperture surrounding the microcolony, thereby enclosing the microcolony and forming a seal between the fluidic transfer tube and the solid growth medium;
controlling the fluidic transfer device to dispense at least a portion of the harvesting buffer such that a least a portion of the dispensed harvesting buffer enters the solid growth medium, thereby liberating microbial cells from the microcolony and forming a suspension comprising the liberated microbial cells; and
controlling the fluidic transfer device to aspirate at least a portion of the suspension.

In some example implementations of the method, during the dispensing of the harvesting buffer and the aspiration of the suspension, a distal end of the fluidic transfer device resides below a surface of the solid growth medium. The distal end of the fluidic transfer device may extend below the surface of the solid growth medium to a depth between 0.1 and 0.6 mm. The distal end of the fluidic transfer device may extend below the surface of the solid growth medium to a depth between 0.1 and 0.4 mm. The distal end of the fluidic transfer device may extend below the surface of the solid growth medium to a depth between 0.1 and 0.6 mm.

A further understanding of the functional and advantageous aspects of the disclosure can be realized by reference to the following detailed description and drawings.

### BRIEF DESCRIPTION OF THE DRAWINGS

Embodiments are described with reference to the accompanying drawings. In the drawings, like reference numbers can indicate identical or functionally similar elements. The drawing in which an element first appears is generally indicated by the left-most digit in the corresponding reference number.

FIG. 1A is a flow chart illustrating an example method of rapidly detecting antimicrobial resistance markers based on harvesting microbial cells from a microcolony grown direct from sample and performing nucleic acid amplification on nucleic acids released from the harvested microbial cells.

FIG. 1B is a flow chart illustrating an example method of rapidly performing a nucleic acid amplification assay panel based on microbial cells harvested from a microcolony grown direct from sample.

FIG. 2 is a flow chart illustrating an example method of rapidly performing sequencing based on microbial cells harvested from a microcolony grown direct from sample.

FIG. 3 presents the average time to positivity and the diameter of microcolony for some microbial species.

FIG. 4A presents the number of bacterial cells harvested from a microcolony incubated for 4 hours after cell suspension spreading. Each data point is averaged over 5 microcolonies.

FIG. 4B schematically presents an example method of performing microcolony harvesting by electrical attraction and repulsion.

FIG. 4C presents the number of bacterial cells harvested from a microcolony incubated for 4 hours after cell suspension spreading, employing the suction and electrical harvesting methods. Each data point is averaged over 5 microcolonies.

FIG. 5 presents images of Gram-stained Escherichia coli ATCC 35218 microcolony (4 hours incubation) harvested by affinity surfaces having different adhering material

FIG. 6 presents images of Gram-stained Staphylococcus aureus ATCC 25923 microcolony (4 hours incubation) harvested by affinity surfaces having different adhering material.

FIG. 7 presents the time to positivity of RT-LAMP assay run on cell suspensions harvested from Staphylococcus aureus and E. coli microcolonies.

FIG. 8 presents the time to positivity of RT-LAMP assay run on cell suspensions harvested from Staphylococcus aureus and E. coli microcolonies which had been grown for 4 hours following spreading.

FIG. 9A presents the time to positivity of RT-LAMP assay run on cell suspensions harvested from microcolonies which had been grown for 4 hours following spreading.

FIG. 9B compares the time to positivity of RT-LAMP assay run on two aliquots of cell suspensions harvested from microcolonies, which had been grown for 4 hours following spreading; one aliquot was subjected to heat lysis but the other aliquot tested without lysis.

FIG. 10A presents the CT values for PCR assays performed on cell suspensions harvested from Staphylococcus aureus ATCC 29223 colonies grown 4-hour or 6 hours after spreading.

FIG. 10B compares the CT values for PCR assays performed on two aliquots of cell suspensions harvested from Escherichia coli and Staphylococcus aureus microcolonies grown 4-hour after spreading.

FIG. 11A illustrates a section of a blood agar plate imaged by an upright reflected-illumination (epi) bright-field (BF) metallurgical microscope with 5× infinite plan objective. One μL of microbial cell suspension, obtained from whole blood which was treated by selectively lysing with a blood lysis reagent composed of saponin and sodium polyanethole sulfonate (SPS), followed by two centrifugal wash cycles, was dispensed on the plate and allowed to air dry before obtaining the microscopic image. The region over which the sample had spread is indicated by 312.

FIG. 11B illustrates a section of a blood agar plate imaged by an upright reflected-illumination (epi) bright-field (BF) metallurgical microscope with 5× infinite plan objective. One μL of microbial cell suspension, obtained from whole blood which was treated by selectively lysing with an alkaline blood lysis reagent including saponin, SPS, Triton-X100, and carbonate-bicarbonate buffer, followed by 2 wash cycles, was dispensed on the plate and allowed to air dry before taking the microscopic image.

FIG. 11C illustrates the blood debris size distribution obtained using the blood lysis reagent employed when processing the sample according to the method described with reference to FIG. 3B. One μL of microbial cell suspension, obtained from whole blood, which was treated by selectively lysing with an alkaline blood lysis reagent including saponin, SPS, Triton-X100, and carbonate-bicarbonate buffer followed by 2 or 4 wash cycles was dispensed on the plate and was allowed to air dry before taking microscopic image by 10× infinite plan objective. The image was analyzed for particle size distribution and the histogram of the particle size distributions was plotted for both 2 wash cycle (left) and 4 wash cycle (right).

FIG. 12 illustrates a section of mini-culture regions (MCRs) formed on agar plates after dispensing of 1 μL of microbial cell suspension obtained by centrifugally separating a whole blood sample spiked with Proteus mirabilis (PM), imaged by a bright-field (BF) metallurgical microscope with 5× infinite plan objectives at time points of 0 hour, 2 hours, 3 hours, and 4 hours following incubation. The arrows indicate some of the PM microcolonies which can be visually discerned relative to the blood lysis debris.

FIG. 13 illustrates example steps for differentiating microbial colonies on the MCRs of FIG. 6 from the blood lysis debris via time-lapse image analysis. Imaging data acquired at different time points (0, 2, 3 and 4 hours after seeding) was spatially aligned (registered) with respect to 0 hour image, followed by a subtraction of the 0 hour image. Intensity features present within the 0 hour image were classified as background (blood lysis debris) while intensity features appearing in the subtracted images were classified as foreground microcolonies).

FIG. 14A is a table presenting the measured growth parameters of seeded ATCC strains of Gram-positive bacteria, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 14B is a table presenting the measured growth parameters of seeded clinical isolates of Gram-positive bacteria, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 14C is a table presenting the measured growth parameters of additional seeded clinical isolates of Gram-positive bacteria, recovered from spiked blood sample via centrifugal separation and subsequent seeding onto agar. The lag time before growth, growth rate, estimated time to positivity and the average time required for the number of cells in a microcolony to reach 104 and 105 CFU are presented for seeded cells growth vs reference growth inside blood culture bottles (liquid culture).

FIG. 14D is a table presenting the measured growth parameters of seeded ATCC strains of Gram-negative bacteria, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 14E is a table presenting the measured growth parameters of seeded clinical isolates of Gram-negative bacteria, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 14F is a table presenting the measured growth parameters of additional seeded clinical isolates of Gram-negative bacteria, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 14G is a table presenting the measured growth parameters of seeded ATCC strains of fungal cells, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 14H is a table presenting the measured growth parameters of seeded clinical isolates of fungi, recovered from spiked blood sample via centrifugal separation and subsequent seeding onto agar. The lag time before growth, growth rate, estimated time to positivity and the average time required for the number of cells in a microcolony to reach 10⁴ and 10⁵ CFU are presented for seeded cells growth vs reference growth inside blood culture bottles (liquid culture).

FIG. 15A plots the number of colony-forming units (CFU) of PM bacterial cells recovered after the centrifugal separation and subsequent seeding onto agar of microbial cells from a spiked whole blood sample at different time points following seeding the final cell suspension and incubating at 37° C. for 4 hours.

FIG. 15B plots the number of CFU of Staphylococcus epidermidis bacterial cells recovered after the centrifugal separation and subsequent seeding onto agar of microbial cells from a spiked whole blood sample at different time points following seeding the final cell suspension and incubating at 37° C. for 4 hours.

FIG. 15C plots the number of CFU of Pseudomonas aeruginosa bacterial cell recovered after the centrifugal separation and subsequent seeding onto agar of microbial cells from a spiked whole blood sample at different time points following seeding the final cell suspension and incubating at 37° C. for 4 hours.

FIG. 15D plots the number of CFU of Escherichia coli bacterial cell recovered after the centrifugal separation and subsequent seeding onto agar of microbial cells from a spiked whole blood sample at different time points following seeding the final cell suspension and incubating at 37° C. for 6 hours.

FIG. 16 illustrates the determination, via optical microscopy, of the positivity of a spiked blood sample for Candida albicans cells (visible inside ovals) after separation from whole blood sample and incubating for 4 hours.

FIG. 17A illustrates the average diameter versus microcolony cell content plot for E. coli. The plot has been fitted with a power law trendline for enabling the estimation of average microcolony diameters, for example, at 10³ and 10⁵ cell content levels.

FIG. 17B plots the average microcolony diameters at 10³ and 10⁵ cell content levels for various pathogenic gram-positive bacteria prevalent in blood stream infection.

FIG. 17C illustrates the average microcolony diameters at 10³ and 10⁵ cell content levels for various pathogenic gram-negative bacteria prevalent in blood stream infection.

FIG. 18 is a flow chart illustrating a whole blood sample preparation method in which a blood lysis reagent containing saponin and sodium polyanethole sulfonate (SPS) is mixed with an alkaline buffer to form an alkaline blood lysis reagent prior to contact with a whole blood sample, after which a separation process is employed to separate microbial cells from the mixture.

FIG. 19 is a flow chart illustrating an improved whole blood sample preparation method in which the whole blood sample is contacted and mixed with a blood lysis reagent containing saponin and SPS to form a first mixture, and where the first mixture is subsequently contacted with an alkaline buffer to obtain a second mixture, after which a separation process is employed to separate microbial cells from the second mixture.

FIG. 20 presents the time dependence of the viscosity (top) and microscopic morphology (bottom) of a mixture obtained from mixing a whole blood sample with a blood lysis reagent containing saponin and SPS, and the effect of contact with an alkaline buffer after a time delay.

FIG. 21 is a table that presents results from an experimental study of the dependence of the particle debris size and surface coverage, after separation and inoculation of a solid growth medium, on the volume of whole blood, the volume of blood lysis reagent, the delay between mixing the saponin and SPS-containing blood lysis reagent with the alkaline buffer, and the pH of the final mixture of blood lysis reagent, whole blood sample, and alkaline buffer.

FIGS. 22A, 22B, 22C and 22D shows microscope images of cell suspensions inoculated onto an agar plate after processing a whole blood sample according to the method illustrated in FIG. 19, with results shown for different concentrations of calcium in the blood lysis reagent.

FIG. 23 is a table presenting the dependence of microcolony detection on time for microcolonies on a solid growth medium after processing a whole blood sample according to the method illustrated in FIG. 19, demonstrating the impact of the concentration of calcium in the blood lysis reagent on microcolony detection and its time dependence.

FIG. 24A is a table showing the composition of example blood lysis reagents employed in experimental demonstrations of cell recovery and molecular amplification.

FIG. 24B shows the processed whole blood sample at the end of the third wash and before removing the top 1.9 mL supernatant. For the case of a blood lysis reagent that is absent of K2EDTA, a reddish pellet is observed at the bottom of the vessel.

FIG. 24C shows microscope images of cell suspensions inoculated onto an agar plate after processing a whole blood sample according to the method illustrated in FIG. 19, with results shown for two different concentrations of K2EDTA in the blood lysis reagent.

FIG. 24D shows the impact of the presence or absence of K2EDTA in the blood lysis reagent on performance of the blood lysis reagent in the collection tube in terms of providing clear eluate.

FIG. 25 illustrates the recovery of different microbial cells from a 2:6 mixture of a blood lysis reagent and whole blood collected in an SPS blood tube.

FIG. 26 illustrates the RT-LAMP inhibition of amplification of Klebsiella pneumoniae lysate by blood matrices obtained from treating 8 mL of un-spiked blood mixed with several example blood lysis reagent formulations. “BLR-1 Ctrl” and “BLR-2 Ctrl” represent amplification of Klebsiella pneumoniae lysate in a clean phosphate buffer. Each bar represents 20 different blood samples (donors) along with the mean (“x”) TTP for all 20 samples, the median, interquartile range, maximum and minimum.

FIG. 27 presents the difference between the time to positivity (TTP) between the RT-LAMP assay testing the cell suspension obtained from treating spiked blood sample and the positive control. Each TTP represents an average of 4 to 6 RT-LAMP reactions.

FIG. 28A schematically illustrates an example suction method for harvesting microcolonies.

FIG. 28B shows microscopic images of a microcolony, before and after harvesting by the suction method of FIG. 28A, without perturbing neighboring microcolonies.

FIG. 28C illustrates the sensitivity of the example suction harvesting method on the position of tip with respect to the gel surface.

FIG. 28D illustrates the reproducibility of the suction method of FIG. 18A for microcolony harvesting.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

### Definitions

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the term “colony” refers to a multiplicity or population of microorganisms that lie in close proximity to each other, that lie on a surface, and that are the clonal descendants, by in situ replication, of an ancestral microorganism.

As used herein, the term “microcolony” refers to a colony having a diameter that is less than 100 μm. In the case of a non-circular microcolony, the diameter is the effective diameter of the microcolony, which may be calculated as the diameter of a circle with the same area as the spatial region associated with microcolony (e.g. calculated as \sqrt{4√Area/π}).

As used herein, the phrase “microcolony growth phase” refers to the time duration during which a growing colony is a microcolony.

As used herein, the phrase “cell suspension” refers to an aqueous medium that contains microbial cells.

As used herein, the phrase “separation process” refers to a process suitable for separating and optionally concentrating microbial cells. Non-limiting examples of separation processes include centrifugation, filtration, immunomagnetic separation and microfluidic separation.

As used herein, the phrase “sample” refers to a liquid or suspension that contains, may contain, or is suspected of containing one or more microbial cells. Non-limiting examples of samples include body fluids such as urine, lymph fluid, cerebrospinal fluid, blood (e.g. whole blood, blood culture, and plasma), sputum, mucus, wound drainage, saliva, lavage, joint fluid, abscess fluid, homogenized tissue suspensions (including, but not limited to, stool, homogenized suspensions of muscle tissue, brain tissue and liver tissue), and any fluid aspirate or tissue extraction of human and/or other mammalian origin. A sample may be processed or unprocessed and may optionally include one or more reagents or growth media.

As used herein, the phrase “blood cells” refers to mammalian cells present in blood, including, but not limited to, red blood cells (erythrocytes), white blood cells (leukocytes) and blood platelets (thrombocytes).

As used herein, the phrase “blood sample” refers to any sample comprising one or more blood cells. Non-limiting examples of blood samples include whole blood samples, blood culture samples, buffy coat samples and platelet samples.

As used herein, the phrase “whole blood” or “whole blood sample” refers to mammalian blood comprising blood plasma and blood cells. “Whole blood” or “a whole blood sample” may include one or more reagents, such as anticoagulation reagents. For example, as described below in some example embodiments of the present disclosure, whole blood may be collected in a sample bottle that may include one or more reagents such as, but not limited to, anticoagulants including SPS (sodium polyanethole sulfonate), EDTA (ethylenediaminetetraacetic acid), sodium citrate and heparin.

As used herein, the phrase “selective lysis” refers to a blood lysis reagent or lysis process whereby the fraction of microbial cells that remain viable following lysis exceeds the fraction of eukaryotic cells that remain viable following lysis, where the eukaryotic cells are associated with the subject from which the sample was collected.

As used herein, the phrase “effective buffer concentration”, when used with reference to a mixture formed by mixing a volume of a sample with a volume of a blood lysis reagent, where the blood lysis reagent includes a buffer system, refers to the product of the buffer concentration of the blood lysis reagent and a ratio formed by dividing the volume of the blood lysis reagent by the sum of the volume of the blood lysis reagent and the volume of the sample. The effective buffer concentration represents the contribution of the blood lysis reagent to the buffer system in the final mixture (i.e. the dilution factor applied to the buffer concentration of the blood lysis reagent) and may be different than the actual buffer concentration in the final mixture due to buffering components present in the sample.

As used herein, the phrases “microbial cell” and “micro-organism” refers to bacteria (e.g. Gram-positive and Gram-negative bacteria, as well as bacterial spores) and unicellular fungi (such as yeast and molds).

As used herein, the phrase “eukaryotic cell” refers to cells originating from an eukaryotic organism excluding fungi, such as animals, in particular animals containing blood, comprising invertebrate animals such as crustaceans and vertebrates. As used herein, “vertebrates” comprise both cold-blooded animals (fish, reptiles, amphibians) and warm-blooded animals (birds and mammals).

As used herein, the phrase “uncultured sample” refers to any sample suspected of containing a viable microbial organism, where the sample has not yet been thermally incubated in a controlled thermal environment suitable for promoting microbial growth. An “uncultured sample” may include growth media. An example of an uncultured sample is a blood culture bottle containing a blood sample, where the blood culture bottle has not yet been subjected to incubation in a thermal incubator.

As used herein, the phrase “nucleic acid amplification” refers to a process for amplifying or multiplying one or more nucleic acid molecules. In some embodiments, the one or more nucleic acid molecules are from a pathogen, such as virus, bacterium or fungal pathogen. The number of nucleic acid molecules can be amplified according to several different nucleic acid amplification modalities, including, but not limited to, polymerase chain reaction (PCR), real-time PCR, strand displacement amplification (SDA), transcription mediated amplification (TMA), quantitative PCR (qPCR), reverse transcription PCR (RT-PCR), loop-mediated isothermal amplification (LAMP), RT-LAMP, strand displacement amplification (SDA), helicase-dependent amplification (HDA), transcription-mediated amplification (TMA), recombinase polymerase amplification (RPA), nucleic acid sequence-based amplification (NASBA), and variations thereof. In some example nucleic acid amplification modalities, nucleic acid amplification may be performed via or employ thermal cycling, while in other example modalities, nucleic acid amplification may be performed via or employ isothermal amplification.

As used herein, the phrase “target nucleic acid” refers to a nucleic acid comprising a target sequence to be amplified and/or detected. Target nucleic acids may be DNA or RNA, and may be either single-stranded or double-stranded. The target nucleic acid may include other sequences besides the target sequence, which may not be amplified.

As used herein, the phrase “target sequence” refers to the particular nucleotide sequence of the target nucleic acid that is to be amplified and/or detected.

As used herein, the phrase “buffer” refers to any solution with a controlled pH that may serve to dissolve a solid substance (e.g., reagent, sample, or combination thereof) or as a diluent to dilute a liquid (e.g., a liquid reagent, liquid sample, or combination thereof; or a solution of a reagent, sample, or combination thereof).

As used herein, a “polymerase” refers to an enzyme which catalyzes the formation of a new nucleic acid molecule (e.g., DNA or RNA) utilizing an existing nucleic acid molecule (e.g., DNA or RNA) as a template to produce a complementary (or substantially complementary) polynucleotide sequence in the new molecule. In some embodiments, a polymerase is a strand displacement polymerase, which catalyzes the displacement of one strand of a DNA double helix before DNA or RNA synthesis occurs.

As used herein, a “primer” refers to a polynucleotide that can serve to initiate a nucleic acid chain extension reaction.

As used herein, a “probe” refers to a polynucleotide that can hybridize (e.g., specifically) to a target sequence in a nucleic acid, under conditions that allow hybridization, thereby allowing detection of the target sequence or amplified nucleic acid.

As used herein, the term “multiplex PCR” refers to a type of PCR where more than one set of primers is included in a reaction allowing one single target, or two or more different targets to be amplified in a single reaction tube. The multiplex PCR can be, for example, a real-time PCR.

As used herein, the phrase “syndromic panel” refers to a multiplex assay that simultaneously detects multiple different pathogens associated with a similar and/or overlapping clinical symptoms.

As used herein, the phrase “solid growth medium” refers to a growth medium which allows micro-organisms to form colonies on its surface. One example of a solid growth medium is a gel, such as an agar-based gel, the gel being a colloidal system in which a porous network of interconnected particles spans the volume of a liquid medium and allows nutrients to diffuse through the medium to become available to the microorganisms.

As used herein, the phrase “host cells” refers to cells derived from the host or subject that are distinct from commensal (e.g., microbial cells which are part of the host microbiome), infectious (e.g., pathogenic microbes) or contaminating cells (e.g., introduced accidentally during sample collection or preparation). The term “host” may refer to a patient or medical subject, which may be a human or non-human mammal.

As used herein, the phrase “saponin” refers to steroidal saponin, triterpenoid saponin and/or a combination thereof.

### Introduction

As described above, the conventional diagnostic workflow for the determination of the causative microbial organism responsible for an infection, and the antimicrobial susceptibility of the causative microbial organism, is fraught with challenges and time delays. Indeed, the time delay incurred prior to the availability of microbial identification and antimicrobial susceptibility results is typically several days, with the consequence that the current standard of care for treating microbial infections is driven predominantly by empiric therapy, with identification and antimicrobial susceptibility results often being delivered too late to be clinically impactful on a patient-specific basis.

The present inventors set out to address these shortcomings, developing a new approach to detection, identification and antimicrobial susceptibility testing of blood samples. In contrast to the conventional workflow that relies on blood culture, the present inventors developed a new approach based on direct growth of microcolonies. In such microcolony-based methods, a blood sample is processed to obtain a concentrated suspension of microbial cells, which is dispensed onto a solid growth medium. The solid growth medium is incubated to grow microbial colonies, which are autonomously detected via microscopy imaging during the early microcolony phase.

In International Patent No. PCT/CA2019/051895, titled “SYSTEMS AND METHODS FOR MICROCOLONY GROWTH AND MICROBIAL CELL CHARACTERIZATION”, which is incorporated herein by reference in its entirety, the present inventors demonstrated that microcolony detection could be achieved in mere hours for a wide variety of bacterial and fungal species. This rapid microcolony-based detection of the presence of infection presented a significant departure and improvement over conventional blood-culture based approaches to detection.

The present inventors also described how microbial cells could be harvested from a growing microcolony and employed for microbial cell identification via MALDI (matrix-assisted laser desorption/ionization) and/or employed for antimicrobial susceptibility testing (AST). Specifically, International Patent No. PCT/CA2019/051895 describes methods in which the size of a growing microcolony is monitored via intermittent microscopy imaging, with microbial cells being harvested from the microcolony once a measure associated with microcolony size is determined to have exceeded a pre-determined size threshold associated with a sufficient quantity of microbial cells for performing MALDI and/or AST. As described in International Patent No. PCT/CA2019/051895, microcolonies have been observed to grow to contain a sufficiently large number of microbial cells for MALDI and/or AST within approximately 5 hours for a wide range of microbial species. With MALDI results being available in under an hour, and with new rapid AST methods being capable of delivering MIC results within a few hours (such as, for example, the local diffusion AST method described by the present inventors in International Patent No. PCT/CA2019/051895), the microcolony-based method could enable the delivery of full phenotypic AST results within a single shift.

The present inventors further developed this concept to include a presumptive identification step, prior to microcolony harvesting, in which dark field images obtained at different time points during the microcolony growth phase could be processed via a machine learning algorithm to provide an initial determination of microbial cell class, such as a genus and/or species determination. Such methods of presumptive identification are disclosed in International Patent Application No. PCT/CA2021/050884, titled “SYSTEMS AND METHODS FOR CLASSIFICATION OF MICROBIAL CELLS GROWN IN MICROCOLONIES”, which is incorporated herein by reference in its entirety. The presumptive determination of the microbial cell class could be employed, for example, as an initial means of identification, and/or, for example, the selection of suitable subset of antimicrobial agents for antimicrobial susceptibility testing.

Accordingly, the direct microcolony-based method previously disclosed by the present inventors in International Patent Application Nos. PCT/CA2019/051895 and PCT/CA2021/050884 provided a complete workflow that was capable of providing (i) rapid detection of the presence of an infection without a few hours of sample collection based on direct observation of microcolonies, (ii) an initial presumptive identification based on image-based classification, (iii) rapid full microbial identification by performing MALDI on microbial cells harvested from a microcolony, and (iv) same-shift phenotypic AST performed on microbial cells harvested from a microcolony (yielding minimum inhibitory concentration results), with a susceptible/intermediate/resistant determination being made based on the AST and MALDI ID results and established clinical breakpoints. This approach therefore appeared to deliver a complete solution to the problems associated with the conventional workflow in microbiology.

However, despite the utility of the aforementioned direct microcolony-based workflow that leveraged MALDI and AST to provide same-shift phenotypic AST from harvested microcolonies, the present inventors recently came to the realization that the direct microcolony-based method could be adapted to provide additional clinical value by facilitating the detection of antimicrobial resistance genes prior to the delivery of the same-shift full phenotypic AST results. Indeed, while the detection of the presence of infection is arguably the most clinically relevant result that can be provided—a result that is indeed rapidly and unambiguously provided by the direct microcolony-based method via the direct detection of growing microcolonies—the next most relevant and clinically actionable information is a determination of whether or not the infectious microbial pathogen exhibits antimicrobial resistance.

Accordingly, while the same-shift full phenotypic AST results would directly address the presence or absence of antimicrobial resistance, the present inventors sought an approach that would facilitate the clear determination of antimicrobial resistance earlier in the workflow, soon after the detection of the presence of infection.

The molecular detection of antimicrobial resistance is conventionally performed using positive blood culture samples that have a very high abundance of microbial cells. Such an approach has been successfully commercialized, for example, as multiplex syndromic blood culture identification panels including the BioFire® FilmArray® BCID panel, the Roche® ePlex® and the Luminex® Veigene® Bloodstream Infection Testing Panels, each of which include genomic resistance markers.

At the other extreme, attempts have been made to employ nucleic acid amplification to directly direct low-abundance molecular pathogens in whole blood samples. For example, T2 Biosystems® has developed the T2Resistance® Panel, which directly detects resistance genes in microbial pathogens separated from whole blood.

The present inventors realized that both approaches to resistance marker detection, that is, the positive blood culture approach and the direct whole blood approach, have significant drawbacks. The positive blood culture approach enjoys the benefit of a high abundance of microbial cells, facilitating clear and unambiguous detection of resistance markers. Furthermore, the positive blood culture approach is cost-efficient in that a determination of positive is already made prior to performing the assay. On the other hand, the positive blood culture approach suffers from the significant time delay that occurs prior to blood culture positivity, with resistance marker results often being delayed until the second day. Accordingly, even though such multiplex syndromic panels are rapid from the perspective of assay time-to-result, the delay in blood culture positive renders them slow and delayed from a clinical perspective.

In contrast, the direct whole blood approach to the detection of resistance markers excels in providing resistance marker results very quickly, often within hours of sample collection. Unfortunately, the direct whole blood approach to the detection of resistance markers suffers from significant drawbacks that ultimately challenge its adoption. One drawback of direct whole blood detection is the scarcity of microbial cells in blood samples, which often contain low microbial concentrations at the 1 CFU/ml level. This low abundance of target places a high burden on the assay technology, pushing sensitivity close to its limit and risking the detection of false positives. Another challenge with whole blood samples is that the target microbial cells are vastly outnumbered by host blood cells, whose nucleic acid content along with cell-free DNA can outnumber bacterial genetic material by 8 or 9 orders of magnitude.

These challenges are compounded by the low prevalence of positive whole blood samples, with positivity rates typically ranging between 5-10%. While such low prevalence inherently results in a high negative predictive value, the low prevalence effectively amplifies the risk of false positive detection. For example, in a direct detection syndromic panel that tests for numerous resistance markers, the net prevalence of detection of a given resistance marker may be less than 1%, since the net prevalence includes both (i) the prevalence of the resistance marker among positive samples and (ii) the prevalence of positive samples among all samples. Accordingly, even a false positivity rate of 1% can lead to a greater number of false positives than true positives, hampering the ability of such a test to be employed clinically to drive early treatment decision making.

### Microcolony-Based Rapid Detection of Antimicrobial Resistance

Aware of the significant drawbacks of both the positive blood culture and the direct whole blood approach to resistance marker detection, the present inventors sought an improved approach that would capture the benefits of both conventional approaches and avoid their drawbacks. In other words, the present inventors sought a new approach to resistance marker detection that would (i) include an initial detection step to determine which samples were positive (filtering out the negative samples), (ii) provide results within a few hours of receiving a sample, and (iii) perform robust detection of sufficiently high abundance of microbial cells to avoid problems with false positives.

The present inventors realized that this goal could be achieved by adapting the direct microcolony-based approach to perform nucleic acid detection and/or sequencing of microbial cells harvested from a microcolony. Such a microcolony-based nucleic acid detection method could be capable of meeting the requirements outlined above. The key to this realization, as will be described in detail below, was the recognition that a microcolony, even at the early stages, provides a highly localized and purified source of microbial cells that can be efficiently harvested for nucleic acid detection and/or sequencing, in the absence of an intervening microbial growth step between harvesting and nucleic acid amplification and/or sequencing.

Indeed, the microcolony-based detection methods described herein facilitate the direct detection of positive samples, in mere hours, based on initial microscopic imaging during microcolony growth, thereby enabling the identification of positive samples prior to nucleic acid detection. The microcolony-based nucleic acid detection method thus solves the “negative problem” and enables nucleic acid detection to be performed cost-effectively on positive samples only.

Moreover, unlike liquid-culture based approaches to the detection, the present microcolony-based methods in which microcolonies are grown from microbial cells obtained from a sample, without intervening liquid-culture-based incubation of the sample, enables the determination of a quantitative measure based on the number of detected microcolonies. For example, the number of detected microcolonies may be employed to infer a severity of an infection, and/or an effectiveness of antimicrobial therapy. This quantitative aspect of the present methods may also be employed to track a severity of infection, or an effectiveness of antimicrobial therapy, over time, based on detection performed on multiple samples at different points in time. This direct aspect of microcolony-based detection may also be beneficial in facilitating a determination of the inherent and true polymicrobial nature of a sample, unperturbed and unbiased by competition that would otherwise occur in a liquid culture environment and which could mask or skew relative concentrations of sub-populations of different types of microbial cells.

Furthermore, as will be described in detail below, the present microcolony-based detection methods enable nucleic acid amplification and/or sequencing to be initiated quickly, soon after microcolony detection, without requiring further growth (without requiring further cell enrichment). Indeed, it will be shown that even very small microcolonies, such as microcolonies with a lateral extent of only 20-50 microns, typically contain ample microbial cells for efficient and robust nucleic acid detection. Given that microcolony detection and harvesting can be performed within a few hours of the initiation of sample processing, and that subsequent nucleic acid amplification steps can often be performed within an hour (with sequencing results being available within 2-3 hours), the microcolony-based resistance marker detection method is expected to be capable of providing resistance marker results within hours of sample collection, well within a single shift.

Additionally, the present inventors realized that the microcolony-based detection method provides significant benefits in terms achieving a level of enrichment and isolation that is unachievable using either of the conventional approaches of positive blood culture detection and direct whole blood detection. Indeed, in the case of positive blood culture detection, the sample is a complex suspension of microbial cells, growth medium, blood debris, and other components of the blood culture medium, such as antimicrobial absorbing resins. Each of the aforementioned commercially available products thus requires complex microbial cell isolation and nucleic acid extraction steps, even with the advantage of a high microbial cell load in the initial sample. In the case of direct whole blood processing, the low prevalence of microbial cells in the blood sample places a high bar on the sample preparation steps that are needed to isolate the microbial cells and extract the nucleic acids. Indeed, the present inventors have found that it can be very challenging to process a sufficiently high volume of whole blood to facilitate detection of single CFU/ml samples, while also achieving high microbial cell recovery, high microbial cell viability, and to achieve sufficient concentration of the separated microbial cells, while also reducing the concentration of nucleic acid interferents. Such challenges are likely responsible for the hindrance and dearth of commercially available whole blood nucleic acid detection and/or sequencing solutions.

In stark contrast to these limitations of the conventional approaches, the present microcolony-based nucleic acid detection method achieves an enhanced level of isolation and concentration by virtue of the microcolony modality. Unlike the conventional direct whole blood nucleic acid amplification approach, which is entirely performed in the liquid phase, the intermediate solid growth step of the microcolony-based nucleic acid detection method effectively provides an enhanced level of spatial and growth-based isolation and concentration.

Indeed, the microcolony-based nucleic acid detection and/or sequencing methods disclosed herein can achieve high spatial isolation and concentration of microbial cells because the microbial cells are localized at, and replicate within, the small microcolony region on a solid growth medium, in stark contrast to liquid-phase methods which the microbial cells are spatially-distributed and replicate across the entire liquid volume of the liquid growth medium.

This spatial isolation and concentration of the microbial cells of a microcolony enables the efficient harvesting of microbial cells for nucleic acid detection and/or sequencing with high purity and isolation relative to background such as host cell debris and other interferents. The power and utility of this spatial isolation and concentration step can be readily understood and appreciated by comparing the purification and isolation that can be achieved with a microcolony to the purification and isolation that is achieved with conventional centrifugation methods. For example, in a conventional centrifugal processing step, a 1 ml liquid sample may be centrifugally processed to separate microbial cells and the supernatant may be removed to leave 100 μl of microbial cell concentrate. After adding a 1 ml dilution buffer, this step may be repeated, thereby concentrating the microbial cells by a factor of 10 and diluting interfering substances by a factor of 10. If two additional dilution and centrifugation steps are performed, the overall concentration factor of the microbial cells remains 10, while the dilution factor increases to 10³.

In the present microcolony-based methods, the spatially localized nature of the growth and concentration enables a much higher degree of separation. For example, considering an example case in which a 100 μL sample containing 10 CFU is spread over a solid growth medium having a diameter of 60 mm, and the growth medium is incubated to support the growth of microcolonies, 10 microcolonies form. If a given microcolony is detected and efficiently harvested when the microcolony diameter is 50 μm, the spatial concentration achieved is [(60 mm)/(0.05 mm)]²~10⁶. Even if the microcolony is harvested with a harvest diameter of 500 μm, the spatial concentration achieved is [(60 mm)/(0.5 mm)]² ~10⁴. This high degree of spatial concentration—and dilution of interferents—is much higher than what can be achieved with the four rounds of centrifugation of the preceding example. Moreover, unlike conventional centrifugation approaches, in which some interferents can continue to sediment during subsequent rounds of washing and centrifugation, impairing the ability to achieve high dilution factors, the spatial isolation of microcolony localization and growth ensures a high purification factor upon harvesting.

When both modalities are combined, that is, initial centrifugal-based concentration (or another initial separation modality such as filtration or microfluidic separation) followed by subsequent microcolony growth, a remarkable degree of isolation and concentration can be achieved.

Moreover, it is noted that the methods of the present disclosure, which involve the preparation, directly from sample, optionally via an intervening separation step, of microcolonies, with nucleic acid amplification and/or sequencing being performed on harvested microcolonies, may be performed without needing the split an initial sample into multiple aliquots, which can hinder the sensitivity of other approaches to the direct processing of samples that rely on sample splitting initial infection detection and subsequent nucleic acid detection or AST, since in the present example methods, infection detection and downstream processing flow directly from the sample, to the detected microcolony, to the harvesting of microbial cells from the detected microcolony.

The present microcolony based methods are also advantageous in separating the microbial cells from antibiotics that may already be present in the initial sample. Indeed, in the case of whole blood samples that are obtained from patients suspected of sepsis, it is common for empiric antimicrobial therapy to be initiated prior to initial phlebotomy. While an antimicrobial concentration may be reduced as a result of an initial separation step to separate microbial cells from the sample (e.g. centrifugation, filtration and/or microfluidic separation), the subsequently inoculation the separated microbial cells onto the solid growth medium may also effective in diluting the concentration of antimicrobial agents initially present in the sample by diffusion of the antimicrobial agents into the solid growth medium, thereby reducing the local concentration of antimicrobial agents during microcolony growth.

Accordingly, various example embodiments of the present disclosure employ microcolony-based growth and detection, and the harvesting of microbial cells from a detected microcolony, while the colony remains in the microcolony phase, to provide a purified and concentrated source of microbial cells for performing downstream assays and applications, such as rapid nucleic acid detection and/or sequencing, for example, for the rapid detection of antimicrobial resistance markers.

### Microcolony-Based Detection of Resistance Markers and Rapid Syndromic Identification Panels

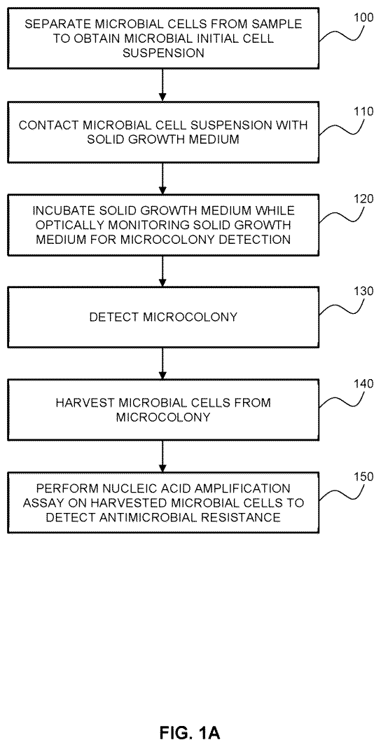

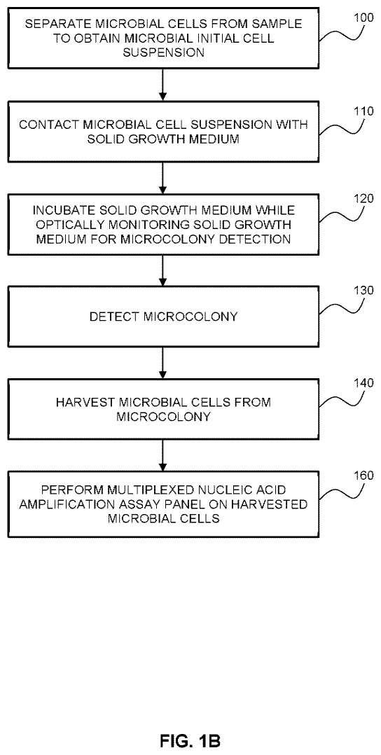

An example method of performing microcolony-based nucleic acid detection is illustrated in FIG. 1A. In step 100, a sample is processed according to a separation method to separate microbial cells from the sample and obtain an initial suspension of microbial cells. In step 110, the suspension of microbial cells is contacted with a solid growth medium (examples of which are described below). The growth medium is incubated to support the growth of microcolonies and optically monitored via microscopy imaging for microcolony detection, as shown at step 120. At step 130, a microcolony is detected and located, which, as demonstrated in the examples provided below, typically occurs within 1-3 hours of inoculation of the solid growth medium. Microbial cells are harvested from a detected microcolony (or a plurality of microcolonies) in step 140 and resuspended to form a concentrated suspension (e.g. resuspended in a buffer). The harvesting step schematically illustrated in FIG. 1A can be performed according to many different approaches, examples of which are described in detail below.

A nucleic acid amplification assay is performed using the harvested and resuspended microbial cells (amplifying nucleic acids released after lysis of the harvested microbial cells) to detect a resistance gene associated with antimicrobial resistance of the microbial cells in step 160, without performing further microbial cell growth after harvesting the microbial cells. A wide range of nucleic acid amplification assays may be performed to determine the presence or absence of at least one antimicrobial resistance gene. In some example implementations, the nucleic acid amplification assay performed on the harvested microbial cells may provide a single result, such as, for example, a nucleic acid amplification assay that targets at least one specific antimicrobial resistance gene of interest, or, for example, a plurality of nucleic acid amplification assays may be performed to detect the presence of a panel of antimicrobial resistance genes. In some example implementations, an antimicrobial resistance panel may target one or more resistance genes exhibited by Gram positive pathogens, such as, but not limited to, mecA, mecC, msrA, erm genes (associated with Staphycocccus), and VanA and VanB genes associated with Enterococcus; and/or one or more resistance genes exhibited by Gram negative pathogens, such as, but not limited to, CTX-M, IMP, KPC, NDM, OXA, VIM genes (associated with Enterobacteriaceae, Pseudomonas, and Acinetobacter).

While FIG. 1A illustrates an example method involving the detection of one or more antimicrobial resistance genes by performing one or more nucleic acid amplification assays based on the microbial cells harvested from a microcolony, in other example embodiments, a target nucleic acid sequence detected by a nucleic acid amplification assay performed based on the microbial cells harvested from the microcolony may characterize an aspect of the microbial cell other than, or in addition to, a presence of an antimicrobial resistance gene.

In some example implementations, a nucleic acid amplification assay performed based on the harvested microbial cells may provide a single assay result, such as, for example, a nucleic acid amplification assay that detects a specific antimicrobial resistance gene of interest, a specific microbial cell kingdom of interest, a specific microbial cell genus of interest, a specific microbial cell species of interest, a specific microbial cell strain of interest, a single Gram status of interest (with the presence or absence of the other Gram status being inferred).

The amplification product, often referred to as amplicons, may be detected by methods known in art, such as turbidity detection, visual detection of binding probes, and fluorescence detection via intercalating dyes or fluorescence probes. In other example methods, amplification products can be separated by size using gel electrophoresis, and visualized using DNA intercalating dyes such as ethidium bromide or SYBR Green. The DNA fragments can be compared to a DNA ladder to determine their size.

FIG. 1B illustrates an example method involving in which a panel of nucleic acid amplification assays are performed based on the microbial cells harvested from the microcolony. Accordingly, in some example implementations, a plurality of nucleic acid amplification assays may be performed based on the harvested microbial cells as an assay panel (e.g. a syndromic panel) configured for the detection of multiple microbial pathogens, with each nucleic acid amplification assay of the assay panel being configured to detect a different identifying characteristic (or type) of the microbial cells, such as, but not limited to, microbial cell kingdom, genus, species, strain, resistance marker, or Gram status (e.g.). In some example implementations, the assay panel may be configured for the detection and identification of a plurality of microbial pathogens. In some example implementations, the assay panel may be configured for the detection and identification of a plurality of microbial pathogens and a plurality of antimicrobial resistance genes. In some example implementations, the assay panel may include one or more replicate assays and/or one or more quality control assays. At least two nucleic acid assays of the assay panel may be spatially multiplexed as separate reactions. At least two nucleic acid assays of the assay panel may be multiplexed within a common reaction. At least two nucleic acid assays of the assay panel may be performed as nested nucleic acid amplification reactions.

A syndromic panel may be configured, for example, to detect a set of pathogens associated with clinical indications such as, but not limited to, bloodstream infections (bacteremia), respiratory infections, urinary tract infections, vaginal and/or sexually transmitted infections, gastrointestinal infections, and antimicrobial resistance.

In some example implementations, the harvested microbial cells are lysed prior to performing the nucleic acid amplification reaction. As described in detail below, the present inventors have found that a wide variety of lysis methods may be employed to successfully detect nucleic acids from harvested microcolonies. In particular, as explained below, the present inventors have found the surprising result that heat lysis may be a sufficient lysis method for a broad range of Gram negative and Gram positive pathogens, unlike the conventional expectation in the art that heat lysis is typically insufficient for the lysis of Gram positive microbial cells.

The present inventors have found that due to the high purity of the harvested microbial cells, after the resuspension of the harvested microbial cells in a buffer (compatible with nucleic acid amplification) and after lysis of the harvested microbial cells within the buffer, the nucleic acids released from the microbial cells may be directly amplified without requiring a nucleic acid extraction or isolation step.

The present inventors have also made the surprising discovery that in some cases, the material from a harvested microcolony may be directly subjected to nucleic acid amplification in the absence of a microbial cell lysis step and in the absence of a nucleic acid extraction or isolation step. Without intending to be limited by theory, it is suspected that the microcolony matrix may include nucleic acids that facilitates nucleic acid detection from the harvested microcolony even the absence of the lysis of microbial cells within the microcolony, and/or that growing microbial cells within the microcolony may be more efficiently lysed during thermal cycles of an amplification reaction, such as, but not-limited to, hot-start PCR.

### Sequencing

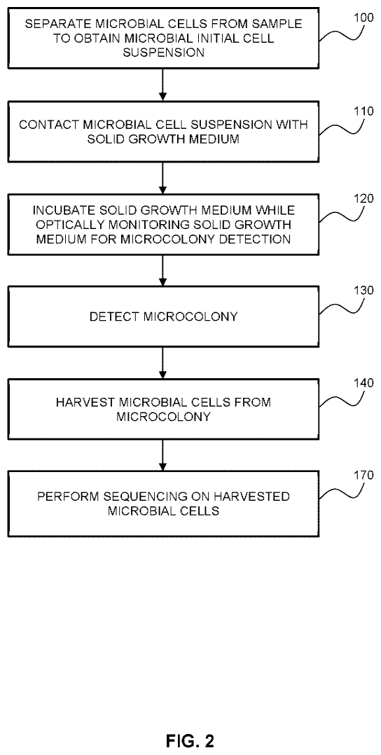

While FIGS. 1A and 1B have disclosed methods for performing rapid nucleic acid detection based on microbial cells harvested from microcolonies, it will be understood that in some example embodiments, the microbial cells harvested from a microcolony may be employed for sequencing, as shown at step 170 of FIG. 2.

In some example embodiments, after detection of microcolony, microbial cells harvested from a microcolony could be employed for sequencing via an intermediate amplification step configured to increase the quantity of nucleic acids to a level that is sufficient for initial sequencing. For example, whole genome sequencing could be initially performed to amplify nucleic acids released from the harvested microbial cells. Without intending to be limited by theory, the present inventors suspect that the purity of the cells harvested from the microcolony, in terms of a reduction in the relative quantity of host DNA when compared to other separation modalities, may improve the fidelity of the resulting amplified nucleic acids, thereby increasing the robustness of the overall sequencing method. Non-limiting methods of whole genome amplification (WGA) include, but are not limited to, polymerase chain reaction (PCR) based methods such as primer extension preamplification PCR (PEP-PCR), degenerate oligonucleotide primer PCR (DOP-PCR), tagged random primer PCR (T-PCR), long and accurate PCR (LA-PCR), ligation-mediated PCR (LM-PCR), interspersed repetitive sequence PCR (IRS-PCR), and isothermal amplification methods such as multiple displacement amplification (MDA), multiple annealing and looping-based amplification cycles (MALBAC), and linear amplification via transposon insertion (LIANTI).

In some example embodiments involving the use of microbial cells harvested from a microcolony for sequencing, the relative purity of the harvested microbial cells may enable sequencing to be performed in the absence of additional steps that would otherwise be employed to enhance the abundance of microbial cell nucleic acids relative to host genomic acids. For example, in some example embodiments, whole genome amplification of nucleic acids obtained from a harvested microcolony may be performed in the absence of blocking primers. Moreover, as described in further detail below, the present example microcolony-based methods enable sequencing-based detection of microbial cells in the absence of dividing a clinical sample.

As will be demonstrated in the examples below, the use of microbial cells harvested from microcolonies addresses several key problems in nucleic acid amplification and sequencing approaches to microbial cell detection. Firstly, as noted above, the ability to rapidly and unambiguously detect microcolonies prior to performing amplification and/or sequencing avoids the cost burden that would otherwise result from the need to perform such assays on negative samples. Secondly, the spatial purification associated with microcolony localization, growth and harvesting substantially reduces or even avoids problems associated with debris and assay inhibitors, enabling harvested microbial cells to be processed via nucleic acid amplification and sequencing assays with minimal or no nucleic acid extraction steps. Thirdly, the initial growth phase prior to microcolony detection and harvesting increases the microbial load, thereby enhancing the sensitivity of the nucleic acid amplification assays and/or reducing the burden in sample preparation for sequencing applications.

In some example implementations, microbial cells harvested from a microcolony are analyzed via next-generation sequencing (NGS). Non-limiting examples of NGS include sequencing-by-synthesis methods, sequencing-by-ligation methods, sequencing-by-hybridization methods, and nanopore sequencing methods.

In some example implementations, after the resuspension of the harvested microbial cells in a buffer and after lysis of the harvested microbial cells within the buffer, the nucleic acids released from the microbial cells may be sequenced without requiring a nucleic acid extraction or isolation step.

Various example steps of the methods disclosed in FIGS. 1A, 1B and FIG. 2, and variations thereof, are described in detail in the forthcoming portion of the present disclosure.

### Inoculation and Incubation of Solid Growth Medium

The microbial cells may be inoculated onto a solid growth medium according to a wide range of inoculation methods. Non-limiting methods of inoculation include: spiral plating (dispensing while spinning the plate and radially moving the dispensing nozzle from center to the periphery as for instance employed by BD Kiestra™ and bioMérieux WASP™), mechanical spreading (dispensing the sample on the plate and spreading by a rod, loop, or bead), spin coating, dispensing in the form of droplets typically having a volume of less than 10 μL. Alternatively, a combination of the said methods may be employed. In one example implementation, the inoculation method is selected such that the preferential accumulation of debris and other contents in peripheries during the liquid drying (commonly known as coffee stain effect) is minimized. Otherwise, an opaque layer may cover microbial cells, thereby impacting their growth and microcolony monitoring.

In some example methods, a sample suspected of containing microbial cells may be directly inoculated onto the solid growth medium. In other example methods, a sample suspected of containing microbial cells may be processed, prior to inoculation of the solid growth medium, to separate and optionally concentrate microbial cells from the sample, thereby providing an initial suspension of microbial cells for inoculation of the solid growth medium. In general, the degree of pre-processing of a sample will depend on both the nature of the sample and the level of purification/dilution of assay interferents in the sample.

By spreading/distributing a suspension of microbial cells over the surface of the solid growth medium, the concentration debris and interfering substances is reduced, and the ratio of microbial/human cells or nucleic acids present in the sample is enhanced. An example method of spreading is disclosed in Example 5.

The inoculated solid growth medium is incubated at an appropriate temperature and environment for promoting microcolony growth. For example, after inoculation, the solid growth medium may be incubated within a controlled temperature range, such as a temperature range between 35-38° C., and a controlled humidity, such as saturation humidity. Examples of suitable solid growth media are described in detail below.

### Microcolony Monitoring and Detection

The growth of microcolonies formed on the solid growth media, following the distribution of the separated microbial cell suspension thereon, may be intermittently monitored according to one or more detection modalities. The monitoring may be performed in-situ, within the incubation environment, or may be performed by temporarily removing the solid growth medium from the incubation environment, performing microcolony detection measurement, and returning the solid growth medium to the incubation environment.

In one example implementation, optical detection may be employed to monitor the growth of one or more microbial cell colonies. For example, in one example implementation, a camera may be employed to image at least a portion of the solid growth media. In another example implementation, an array of photodetectors may be employed in the absence of an imaging element to obtain an image of one or more microbial cell colonies, where a growth surface associated with the colonies is located in sufficient proximity to the array of photodetectors to form an image thereon upon illumination thereof. In implementations in which the field of view of the optical system is less than the spatial extent of the solid growth media, the optical system may be scanned relative to the solid growth media, or vice versa, in order to facilitate the optical interrogation of the entire solid growth media surface, or a desired subset thereof. Example methods of microcolony monitoring are disclosed in International Patent Application Nos. PCT/CA2019/051895 and PCT/CA2021/050884.

An image may be processed using known image processing algorithms to identify microcolonies and to optionally estimate one or more dimensional measures of an identified microcolony. For example, publicly available software, such as the ImageJ/Fiji program, may be employed. In one example method, after converting an image into a greyscale image, the greyscale image may be binarized by applying local adaptive image thresholding according to Phansalkar method, based on histogram analysis of intensity levels. Adaptive image segmentation may then be employed according to Phansalkar method, with dimension constrains that partition an image into segments for microcolony identification. Optional further analysis of the identified segments may then be employed to determine metrics of interest associated with a microcolony (e.g. circularity, area, major axis, and minor axis). There are many different example algorithms that can be used to calculate the threshold in a bias-free manner. The Phansalkar thresholding method is a modification of Sauvola's thresholding method optimized for low contrast images [Phansalskar, N; More, S & Sabale, A et al. (2011), “Adaptive local thresholding for detection of nuclei in diversity stained cytology images.”, International Conference on Communications and Signal Processing (ICCSP): 218-220, doi:10.1109/ICCSP.2011.5739305]. Other example methods include the Bernsen, Contrast, Mean, Median, MidGrey, Niblack, Otsu and Sauvola methods. It will be understood that a wide variety of registration methods may be employed to perform image registration, including, but not limited to, feature-based, intensity-based, and nonrigid registration algorithms. Examples of suitable feature-based algorithms include the SURF (Speeded Up Robust Features) and SIFT (Scale-invariant feature transform) methods.

In one example implementation, image registration may be performed via an intensity-based algorithm as follows. The algorithm transforms the moving image (image acquired at a later time point) so that it is spatially registered with the fixed/reference image (image acquired at a later time point). Based on the set-up, the type of transformation to perform may be defined as ‘rigid’ or ‘affine’. According to the simplified definition, the algorithm internally builds a multi-resolution pyramid in memory (with a user specified pyramid level) and solves an optimization problem on each level of the pyramid. In other words, the algorithm builds an image pyramid that has N levels (e.g. N=5). At each pyramid level the image dimensions are decreased by a factor of 2. Optimization starts at the coarsest level of the pyramid and continues until either user-permitted number of iterations is reached, or until the optimizer converges attempting to refine the current transformation estimate on the following pyramid level.

Alternatively, other image alignment technics such as SIFT, BRIEF, ORB, AKAZE, may be used. Moreover, in cases of reduced feature due to blurring by excessive amount of debris, with the feature extraction in frequency domain is preferred. One exemplary implementation is presented in Example 4C.

The present example time-lapse imaging method is a semi-quantitative imaging technique in which a series of images of the same scene (or approximately the same scene) are taken at different time points to capture the dynamical changes, while a static component is classified as background and can be removed. Current approaches for dynamic profiling of microcolonies rely on assumption of a static background and illumination. However, such a technique may be subject to a number of processing variations if spectral or spatial characteristics of debris or surface of the solid growth media are not static. Firstly, it is noted that a suitable environment should be provided that permits the microcolonies to remain viable while the surface is not substantially aging (evaporation of liquid from the surface of the solid growth media associated with changes of the dimensions of the debris and its displacement) during the acquisition of the images.

The determination of the colony cell content (e.g. the determination of whether or not a sufficient colony size and/or cell count has been achieved for nucleic acid amplification and/or sequencing can be achieved by different approaches. In one example implementation, one or more geometrical or optical properties associated with a colony (such as, but not limited to, radius/area or scattering/reflected/transmitted intensity, as determined from image processing methods, such as image segmentation) may be processed to determine whether or not a sufficient number of microbial cells reside within the colony for subsequent processing, based on a comparison with reference data associating the one or more geometrical properties with microbial cell count. In another example, a neural network may be employed to determine, based on the imaging of a given microbial cell colony, whether or not a sufficient number of microbial cells reside within the colony for subsequent testing, where the neural network is trained based on images of reference strains having known associated cell counts (or, for example, a known binary determination of whether or not a sufficient number of microbial cells is present in the colony for a given type of subsequent testing). In some example implementations, the determination of whether or not a sufficient number of microbial cells resides within a given colony may be determined, in part, based on a detected or inferred identity of one or more taxonomic classes of the colony (e.g. Gram status, genus, family, species, strain, etc.).

It will be understood that optical imaging is but one example detection modality for monitoring the growth of microbial cells, and that other detection modalities, such as electrical impedance, the detection of volatile organic compounds associated with microbial cell growth, or calorimetry may be employed in the alternative.

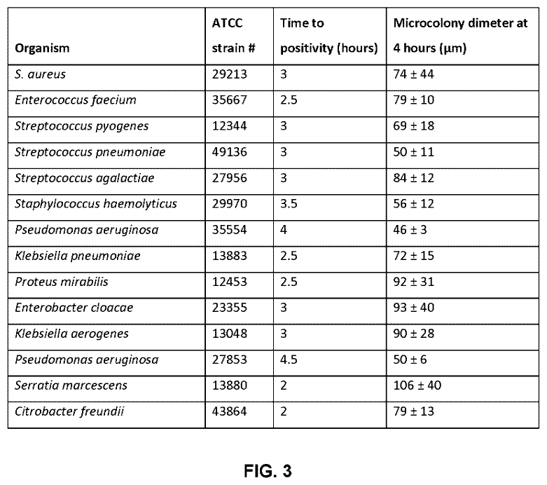

An example experimental determination of the correlation between microcolony size and microbial cell count was conducted as per Examples 1-6 (described below). To find the average time to positivity, microbial cell suspensions with ~100 CFU of different microbial cell types were inoculated on to solid growth medium, and incubated, and intermittent microscopy imaging was performed. The “time to detection” (TTD) was defined as the time after which no new microcolonies were detected with further incubation. The measured time to detection for different bacterial cells is presented in FIG. 3. In addition, the average diameter of microcolonies at 4 hours of incubation is included in the table.

### Microcolony Detection and Size Threshold for Nucleic Acid Amplification

The present inventors have found that for all microbial species tested to date, by the time a microcolony is detectable via microscopy to have a diameter of 20-50 microns, the microcolony has a high microbial cell count to facilitate nucleic acid amplification after harvesting (the present inventors have found that positivity can be detected when the diameter is approximately 20 microns for cell suspensions from whole blood, and that microbial cells harvested from such small microcolonies surprisingly have sufficient biomass a molecular amplification assay). Accordingly, in some example embodiments, a microcolony may be deemed to be ready for harvesting once the microcolony has been detected.

In one example embodiment involving optical imaging of microcolonies grown on solid growth media, the directly or indirectly measured colony dimensions or size may be employed, optionally along with more properties of a given imaged colony (such as the type of microbial cells in a colony; e.g. genus, family, species or strain) to estimate the number of the microbial cells in the colony and/or determine when the incubation can be terminated and the microcolony is harvested for nucleic acid amplification and/or sequencing. The determination of a suitable growth time to achieve a desired number of microbial cells may vary depending on the cell type (e.g. genus, family, species, strain).

The relationship between the microbial cell type and the time to reach a sufficient cell count for subsequent nucleic acid amplification may be established according to a lookup table. For example, an automated system may employ optical image processing to determine the class of cells (e.g. an inferred or estimated microbial species) associated with a given colony and then employ a pre-determined relationship (e.g. a lookup table or a predetermined functional relationship) to determine, for example, a suitable time at which a sufficient number of microbial cells are present in one or more colonies for subsequent processing, or, for example a suitable size measure (e.g. radius or other spatial measure) of the colony for which a sufficient number of microbial cells are present in one or more colonies for subsequent processing. In some example embodiments, multiple criteria involving both a colony size measure and time may be correlated with the microbial cell class in order to estimate when a sufficient number of microbial cells reside within a colony.

### Microcolony Detection and Size Threshold for Sequencing

In some example embodiments involving sequencing, a detected microcolony may be monitored until its size is deemed to be sufficiently large for sequencing in the absence of an initial amplification step prior to library preparation. For example, experimental studies may be conducted to determine a relationship between colony size and quantity of a given type of nucleic acid (e.g. genomic DNA), thereby enabling the determination of a suitable colony size threshold for a given sequencing biomass constraint (e.g. 50 ng, 100 ng, or 200 ng) or, for example, a given sequencing CFU number constraint (e.g. 10⁶ or 10⁷ cells). This threshold may optionally be determined on a species (or other suitable taxonomic rank) level, with the species (or other suitable taxonomic rank being initial determined presumptively via optical microscopy imaging, as disclosed, for example, in International Patent Application No. PCT/CA2021/050884, or, for example, by harvesting microbial cells from a second growing microcolony, and performing an multiplexed nucleic acid amplification identification panel, as per the example embodiments described above, or variations thereof.

The threshold of biomass sufficiency for sequencing library preparation in the absence of initial amplification may occur, for at least some types of microbial cells, after a colony has grown beyond the microcolony stage (e.g. having a diameter, that exceeds 100 μm). Accordingly, while in some example implementations a microcolony may be harvested for sequencing during the microcolony phase (e.g. when initial amplification is to be performed), in other example implementations, a colony that is no longer a microcolony may be harvested for sequencing, for example, after a diameter (or effective diameter) of the colony exceeds 100 μm but is less than 150 μm, or after a diameter of the colony exceeds 100 μm but is less than 200 μm, or after a diameter of the colony exceeds 100 μm but is less than 250 μm, or after a diameter of the colony exceeds 100 μm but is less than 300 μm, or after a diameter of the colony exceeds 100 μm but is less than 350 μm, or after a diameter of the colony exceeds 100 μm but is less than 400 μm, or after a diameter of the colony exceeds 100 μm but is less than 450 μm, or after a diameter of the colony exceeds 100 μm but is less than 500 μm.

### Harvesting

Microbial cells may be harvested, for any downstream assay, application or purpose, from a solid growth media according to a wide range of harvesting methods. Harvesting may be automated, performed manually, or performed in a semi-automated manner. The detected location of a given microcolony is employed to facilitate harvesting, for example, to direct an autonomous (e.g. robotic harvesting step), or to indicate (e.g. via illumination or via annotation on a display showing an image) the microcolony location. Harvested microbial cells may be processed such that they are provided in a form that is suitable for a downstream application, such as, but not limited to, nucleic acid amplification, sequencing, MALDI-based identification, other identification modalities such as Raman spectroscopy, and antimicrobial susceptibility testing. In some example implementations, the harvested microbial cells may be diluted or concentrated. In some example implementations, the harvested microbial cells may be combined with a liquid to form a suspension which may optionally be diluted or concentrated, and optionally aliquoted, prior to performing a downstream application such as one or more nucleic acid amplification assays or sequencing methods.

Various non-limiting example methods of harvesting microcolonies are described in the following sections, and in Example 8 below. It will be understood that different harvesting methods may be suitable, advantageous or preferable for different applications, for example, due to ease of implementation, the ability to perform localized harvesting (and avoid perturbation to the microcolonies adjacent to the target microcolony), and, for example, a required or desired harvesting efficiency.

In one example embodiment, having identified the location of the colonies, the colonies may be manually harvested by biopsy punch, inoculation loop or sterile cotton swabs. In another some example embodiments, having identified the location of the colonies, the colonies may be robotically harvested.

For instance, using a circular instrument, similar to a biopsy punch, a small section of solid growth media may be removed with the microcolony. US Patent Publication No. 2018/0284146 has described a device that is equipped with a platform for holding a culture plate and a movable robotic arm having a pick tool which can be lowered to pick colonies from the plate. In another part of the device, a sterile tube, containing a suspension media, is stored. The pick tool, after picking a part of the colony, moves and transfers the picked colony to the sterile tube. Optionally, the tool may be equipped with a sonicator (ultrasound transducer) or vortex mixer for more efficient release of the harvested microbial cells. In some example implementations, a pipette tip may be employed as a biopsy device for removing a section of culture medium that contains a microcolony.

Another example method of harvesting a microcolony is the scratch harvesting method. This method employs a sterile tool (inoculating loop or spatula) to scrape bacteria off the solid growth media. The tool is then dipped inside a tube containing a clean buffer (e.g., phosphate buffer) and agitated to release the harvested cells.

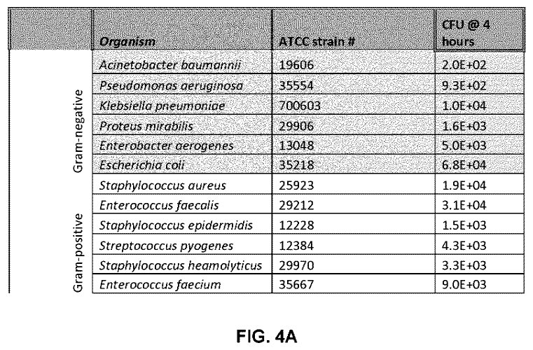

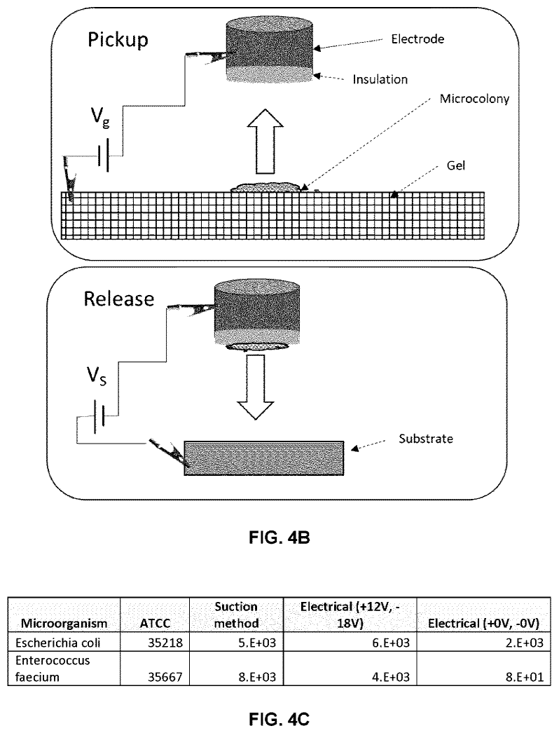

Yet another example microcolony harvesting method is the washing method of harvesting, in which microbial cells are removed from gel surface with a buffer (preferably a buffer that is compatible with a downstream assay or other downstream application). In some example implementations of the washing method, a buffer is dispensed onto the microcolony on the gel surface, and after dispensing of the buffer, the microcolony surface is mechanically disrupted (e.g. scraped, optionally using the dispensing device), such that the microbial cells are brought into suspension form within the buffer. The suspension is then aspirated, removing the microbial cells. An example evaluating the performance of the washing method is described in Example 8B. The colony count after microcolony harvesting microcolonies of different bacterial cell types, after 4 hours of growth post-inoculation, is shown in FIG. 4A, showing the recovery of a large number of microbial cells via the harvesting method after only 4 hours of microcolony growth. Each harvested microcolony cell count corresponds to an average of over 5 microcolonies and harvesting was performed using the “washing” method described in Example 8B. The present inventors also repeated harvesting using three other methods of Example 8 and found that similar cell counts were obtained as in the present example that employed the washing method.

In some example implementations, harvesting is performed by contacting a microcolony with a surface that has been treated to exhibit affinity for microbial cell harvesting/capture, and such surfaces are henceforth referred to as “affinity surfaces”. In some example embodiments, harvesting by contact with an affinity surface may be performed by gently contacting a harvesting tip with the microcolony and having the microbial cells transferred to the tip and immobilize it by forces such as electrical attraction. The harvesting tip having the affinity surface may be, for example, a flat surface (an affinity surface) that is substantially larger than the area of a microcolony and having affinity to microbial cell collection/capture.

The area of the affinity surface may be selected such that it can spatially envelop the microcolony when accounting for locating tolerances of the harvesting device, or based on measured user tolerances when manual harvesting is performed. However, it may be beneficial to limit the size (spatial extent) of the affinity surface area, relative to the size of a microcolony to be harvested, so as to avoid the transfer, with the microbial cells, of excessive amount of residual debris on the solid growth medium surface, or the transfer of an excessive amount of the growth medium as part of the harvested sample. Limiting the size of the affinity surface may also be beneficial in helping to ensure sufficient contact between the affinity surface and the microcolony when the affinity surface is exactly parallel to the surface of the solid growth medium. In some example embodiments the area of the affinity surface is less than 10 mm², less than 5 mm², less than 4 mm², less than 3 mm², less than 2 mm², less than 1 mm², less than 0.5 mm², less than 0.1 mm², and less than 0.05 mm². In some example embodiments the area of the affinity surface is between 1 mm² and 10 mm², between 1 mm² and 5 mm², between 1 mm² and 4 mm², between 1 mm² and 3 mm², between 0.5 mm² and 2 mm², between 0.1 mm² and 1 mm², between 0.0.5 mm² and 0.5 mm², and between 0.05 mm² and 0.1 mm².

In some example implementations, the affinity surface may be chemically treated. In other example embodiments, the affinity surface may be coated with adherent material. In some example embodiments, an adherent material may be coated on a chemically treated surface. The affinity-assisted harvesting of microbial cells, in which microbial cells are transfers to the affinity surface, may be facilitated, for example, via nonspecific adsorption and/or physical entrapment of microbial cells on the surface.

Examples of appropriate coated surfaces suitable for forming an affinity surface include Fisherbrand™ Superfrost™ Plus Microscope Slides (Control SF), PDL (Poly-D-Lysine), PLL (Poly-L-Lysine), Rat tail Collagen I (Collagen), Mouse Laminin (Laminin), Fibronectin on PDL layer (Fibronectin), and Gelatin produced by Neuvitro Corporation, or positively charged slides from Walter Products Inc.

Another example of a harvesting method is the suction method, which is performed as follows. A harvesting buffer is aspirated into a pipette tip or another suitable fluidic dispensing device, such as a liquid transfer tube connected to a pump or aspiration device (for example, as commonly used in an automated liquid handing system). The tip is moved above the microcolony and bought downward to contact the gel surface surrounding the microcolony, such that a fluidic seal is formed between the tip and the gel, enclosing the microcolony, without rupturing the gel. The harvesting buffer is then dispensed onto, and into, the gel, such that at least a portion of the harvesting liquid penetrates the gel surface. The dispensed harvesting buffer is then aspirated to draw the microbial cells liberated from microcolony into the tip, forming an aspirated suspension. The harvesting suspension, containing the microbial cells, may then be dispensed to a location or container of choice.

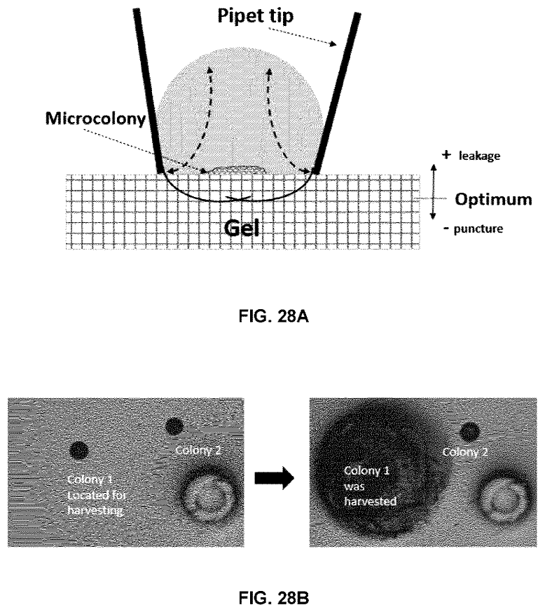

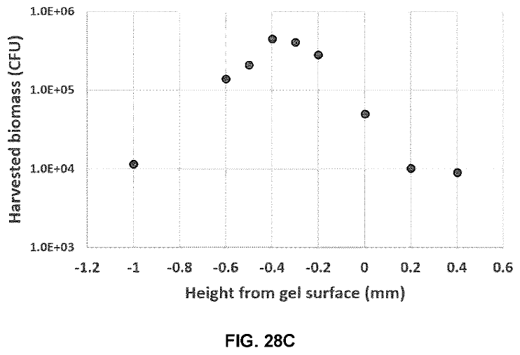

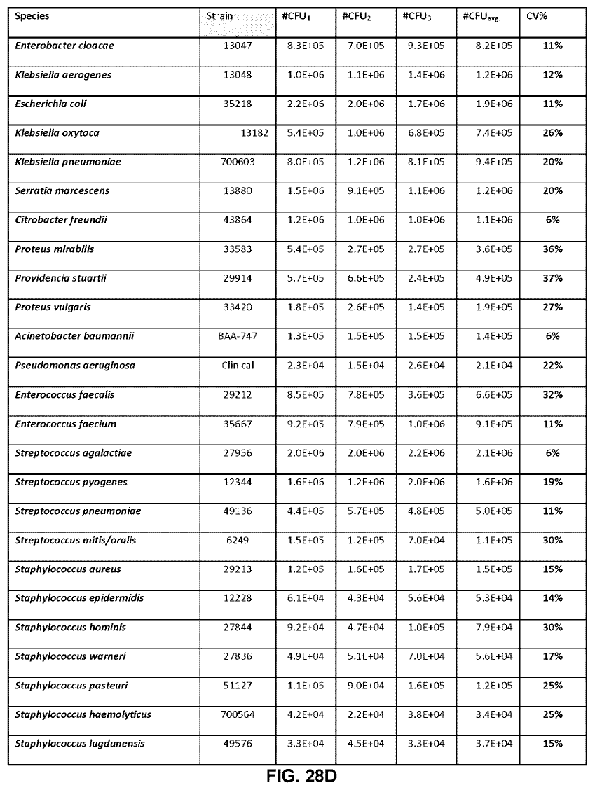

The suction method is schematically illustrated in FIG. 28A. As can be seen in the figure, a seal is formed between the distal open end of the tip and the surface of the gel, enabling the harvesting buffer to penetrate, at least in part, into the gel during the initial dispensing operation. In some example implementations, the tip is pressed into the gel, such that a distal end of the tip penetrates the gel and extends into the gel, thereby increasing the effectiveness of the seal and potentially improving the ability of the harvesting buffer to penetrate and extend into the gel during the dispensing operation.

The present inventors have found that the efficiency of microcolony harvesting by the suction method depends on the position of the distal end of the harvesting tip with respect to the gel surface. To illustrate and investigate this dependence, microcolonies of Staphylococcus aureus ATCC 29213, which had been grown for 5 hours on a blood agar gel at 37° C., were harvested. The harvesting was performed following the protocol described in Example 8D, with the exception that in step 6, the distance of tip from the gel surface was set as a free parameter. The harvesting process was repeated three times at each height and serial dilutions of the harvested microcolonies were plated and incubated overnight for biomass determination.

The average of the harvested biomass as a function of tip height is presented in FIG. 28 C. As it is seen, the harvesting efficiency has a maximum in the range of [−0.6 mm, −0.2 mm]. This result illustrates the benefit of sealing the tip-gel interface without rupturing the gel. It is understood that if the positioning accuracy was sufficiently high, lowering by 0.1 mm or even less could accomplish the task. Accordingly, in some example embodiments, the suction harvesting method may be employed by introducing the distal end of the tip into and below the gel surface by an amount that ranges between 0 and 0.2 mm, 0 and 0.4 mm, 0 and 0.6 mm, 0 and 0.8 mm, 0 and 1.0 mm, or, for example, between 0.1 and 0.2 mm, 0.1 and 0.4 mm, 0.1 and 0.6 mm, 0.1 and 0.8 mm, and 0.1 and 1.0 mm, or, for example, between 0.2 and 0.4 mm, 0.2 and 0.6 mm, 0.2 and 0.8 mm, and 0.2 and 1.0 mm.

The present inventors have found that the suction method is efficient, easy to monitor, and enables localization of the microcolony during harvesting and concentration of the harvested cells, which can be beneficial when the microbial cells are intended to be suspended in a small volume of liquid (e.g. a resuspension buffer).

In order to illustrate the reliability of the suction method of harvesting, experiments were performed according to the method of Example 8D, for which the tip is lowered by 0.3 mm after making contact with the gel surface. The measured harvested biomass, averaged over three microcolonies, for different pathogenic bacterial cells, is presented in FIG. 28D. As is observed, the data indicates good repeatability of the microcolony harvesting approach by taking into account the fact that the observed coefficient of variation (CV) values can easily be justified by expected variations in the growth rate of from one microcolony to another.

It will be understood that many different example methods and/or mechanisms may be employed to control the height of the distal end of the tip relative to the gel surface, for example, to ensure a prescribed insertion depth of the distal end of the tip relative to the gel surface. For example, if the gel surface is known to have a consistent height among different gel supports that are employed for microcolony growth, an initial calibration between tip height and gel surface may be employed to position the tip relative to the gel in an open-loop manner. In another example, closed-loop positioning may be employed, for example, using feedback from an electrically conductive pipette tip to enable a determination of contact with the gel surface (as described, for example, in Example 8D), or, for example, using feedback from a depth sensor or a force sensor or pressure sensor.

The present inventors have found that the present harvesting methods, and the example implementations described in Example 8 below, are suitable for extracting microbial cells and performing downstream applications, such as nucleic acid amplification and/or sequencing, as demonstrated below. However, in some cases, the harvesting process may cause local damage to the solid growth medium, such that further monitoring of any residual cells with any unharvested portion of the microcolony is impeded.

In harvesting methods other than the biopsy method, the local surface of the solid growth medium is less likely to be damaged by harvesting and continued post-harvest monitoring of the growth of residual microbial cells may be feasible. In addition, the likelihood of damaging the gel surface may be further reduced by employing higher gel concentrations as taught by the methods of Example 3. In one embodiment the gel concentration is selected to be between 1.35% and 2.0%, or between 1.35% and 2.5%. In another embodiment the gel concentration is selected to be between 1.5% and 2.0%, or between 1.5% and 2.5%. In some applications, further growth of the microcolony, after harvesting for nucleic acid amplification or sequencing, may be useful to provide microbial cells for other downstream applications, such as, but not limited to, identification with MALDI and/or antimicrobial susceptibility tests (AST).

Moreover, in some applications, resuspending the microcolony in liquid media may not be desirable. For instance, while performing tests such as microbial identification by MALDI, the dispensed cell suspension may be impacted by the “coffee stain effect” during drying, such that the cell content has a non-homogenous spatial distribution over the resulting spot. This may lower the performance of the modality. Microcolony harvesting by affinity capture may be beneficial in alleviating these issues.

In another example embodiment, electrical forces are employed to perform microcolony harvesting. This example method is illustrated in FIG. 4B, and described as follows. Bacterial cell walls and membranes contain numerous proteins, lipid molecules, teichoic acids, and lipopolysaccharides that contribute to its characteristic negative charge at pH values above 4. Accordingly, in the presence of an electric field, for instance induced in the vicinity of an electrode, bacterial cells will be pulled towards a positive polarity.

During the microcolony pickup stage of a harvesting operation, an elongate electrode, preferably with an electrically isolated distal tip (e.g. a pin with an electrically isolating layer formed or provided thereon), with positive polarity (V_{g}) applied thereto, is brought into proximity of the microcolony. The electrode may have a flat distal tip (with an electrical insulating layer formed or applied thereon) having cross-sectional lateral dimensions of approximately 1 mm or less.

During the release stage, the polarity of the electrode is made negative with respect to a substrate onto or into which the microcolony is to be dispensed (Vₛ), thereby releasing the harvested bacterial cells. The magnitude of both V_{g} and Vₛ may vary depending on several factors, such as, for example, the microbial cell species, the electrode shape, and the medium from on onto which the microcolony is to be harvested.

The present inventors have found that a voltage ranging from 3 to 50 volts can be suitable for efficient microbial cell transfer (with higher voltages also being optional, provided that breakdown can be avoided). For isolation, a thin layer of dielectric material may be employed. In one implementation, 1 Mil Kapton® HN film with silicone is used. In another implementation, the electrode is aluminum and the isolation layer is aluminum oxide which is generated through anodization procedure. While contact appears with the substrate appears to be beneficial in achieving efficient electrostatic microbial cell transfer, in some example implementations, one or both of the collect and release operations may be performed in the absence of contact with the substrate, provided that the distal tip of the isolated electrode is brought in sufficiently close proximity to the substrate.

In order to illustrate the performance of electrical microcolony harvesting, 4-hour-old microcolonies were prepared of a representative Gram-negative bacteria (Escherichia coli) and a representative Gram-positive bacteria (Enterococcus faecium). Five microcolonies of each bacteria were then harvested employing the suction method of Example 8D as a reference.

Although the example electrical microcolony harvesting method has been described and demonstrated based on the harvesting of bacterial microbial colonies, it will be understood that the present electrical methods may also be performed for and/or adapted to the harvesting of fungal microcolonies.

While many of the present example embodiments have focused on the harvesting of a single microcolony, it will be understood that multiple microcolonies may be harvested and the microbial cells from the multiple microcolonies may be processed, for example, via nucleic acid amplification and/or sequencing. In some example implementations, microbial cells from multiple microcolonies may be processed collectively, for example, to provide a larger biomass. For example, microbial cells harvested from multiple microcolonies may be combined (pooled) to provide sufficient biomass for a given downstream application, such as sequencing, with or without amplification prior to library preparation.

In other example implementations, microbial cells from different harvested microcolonies may be separately processed, for example, via nucleic acid amplification and/or sequencing, to provide microcolony-specific results. Such microcolony-specific processing may be beneficial, for example, to detect and identify different cell types among multiple microcolonies, such as due to polymicrobial infections, or, for example, due to contaminant pathogens. Microcolony-specific processing may be beneficial and/or to detect heteroresistance among multiple growing colonies.

The selection of a given microcolony for harvesting may be determined based on imaging of the microcolonies prior to harvesting. The selection of a given microcolony for harvesting may be determined based on properties or features of the microcolonies as determined by imaging prior to harvesting. Furthermore, the selection of a given microcolony harvesting method, and/or one or more parameters of a given microcolony method (for example, a suitable harvesting buffer, a suitable volume of harvesting buffer, a suitable electrical harvesting voltage, a suitable contact time interval, a suitable time duration between dispensing a harvesting buffer and aspirating a suspension containing liberated microbial cells from a microcolony, and/or a suitable tip depth for suction-based harvesting), may be made based on the properties or features of a given microcolony (for example, a class or classification of microcolony type based on a machine learning algorithm processing microcolony images).

In some example embodiments, prior to harvesting microbial cells from a second microcolony, a phenotypic correspondence may be established between the first colony and the second colony. This phenotypic correspondence may be established, for example, by comparing classes associated with the two microcolonies, or, for example, comparing optical images or optical signals detected from the two microcolonies. For example, phenotypic correspondence may be established by a machine learning algorithm that classifies the two microcolonies, based on images of the microcolonies, as being members of a common class. A lack of detected phenotypic correspondence may be employed to identify polymicrobial colonies, as described in International Patent Nos. PCT/CA2019/051895 and PCT/CA2021/050884.

### Samples

The methods of the present disclosure may be employed to process a wide variety of sample types, including, but not limited to, urine, lymph fluid, cerebrospinal fluid, blood (e.g. whole blood, blood culture, and plasma), sputum, mucus, wound drainage, saliva, lavage, joint fluid, abscess fluid, homogenized tissue suspensions (including, but not limited to, stool, homogenized suspensions of muscle tissue, brain tissue and liver tissue), and any fluid aspirate or tissue extraction of human and/or other mammalian origin.

In some embodiments, the clinical sample comprises a quantity of DNA residing between 0.1 and 10 microbial genomes per milliliter, or between 0.1 and 10² microbial genomes per milliliter, or between 0.1 and 10³ microbial genomes per milliliter. In some embodiments, the clinical sample comprises a quantity of microbial cells residing between 0.1 and 10 CFU/ml, or between 0.1 and 10² CFU/ml, or between 0.1 and 10³ CFU/ml.

In some cases, the sample may be sufficiently free from debris and potential interferents to facilitate direct inoculation of the solid growth medium. For example, this may be possible with sample types such as, but not limited to, urine.

### Initial Separation of Microbial Cells from Sample Prior to Inoculation of Solid Growth Medium

In many cases, the sample matrix and/or sample volume may necessitate the separation and/or concentration of microbial cells prior to inoculation onto the solid growth medium. This initial processing step may include the lysis of host cells, such as host blood cells. For example, in the case of a whole blood sample, following an initial lysis of host red blood cells, any one or more of a number of microbial cell separation techniques may be employed to provide a suspension of microbial cells suitable for inoculation of the solid growth medium. Non-limiting examples of such methods include lysis-centrifugation, filtration and microfluidic flow-based separation.

A factor which may limit the detection of microcolonies is the size and density of surface artefacts (the background) that are observable via microscopy after contacting the treated sample with the solid growth medium, where such surface artefacts are not representative of microbial cells or microbial cell colonies. Two example types of such surface artefacts that contribute to a background include (i) surface inhomogeneities of the gel surface and (i) lysis debris particles that remain after the lysis of the sample, such as lysis debris particles resulting from the digestion of blood cells, which persist in the sample after centrifugal washing.

The surface density of the first type of artefact, namely gel surface inhomogeneities, can be reduced through controlled fabrication of the gel. An example non-limiting method for preparing gels with a low density of surface inhomogeneities is described Example 3 below.

The density of the second type of artefacts, namely debris particles originating from the sample, can be lowered by the use of a suitable initial host cell lysis reagent and a suitable method of separation of microbial cells. Various examples of such methods are described below.

### Blood Lysis Reagents for Appropriate Digestion and Reduction of Surface Debris

In the case of blood, the present inventors have found that it can be important to ensure sufficient lysis and digestion of host blood cells in order to obtain a microbial cells suspension (e.g. following centrifugation or filtration) that results in a sufficiently low spatial density of surface artefacts after inoculation onto the solid growth medium. The size and amount of the second type of artefacts, namely lysis debris particles may vary from one whole blood sample to another and has been found to depend on the composition of the blood lysis reagent (BLR).

Examples of blood lysis reagents for the separation of microbial cells from whole blood samples are disclosed in International Patent Application No. PCT/CA2013/000992, titled “APPARATUS AND METHOD FOR EXTRACTING MICROBIAL CELLS” and in International Patent No. PCT/CA2019/050716, titled “METHODS AND COMPOSITIONS FOR THE SELECTIVE LYSIS OF BLOOD CELLS AND SEPARATION OF MICROBIAL CELLS”, the latter of which is incorporated herein by reference in its entirety.

International Patent Application No. PCT/CA2013/000992 discloses a number of different blood lysis reagent compositions that may be employed for the digestion of blood components prior to centrifugation. As noted above, the presence of the blood lysis reagent causes the selective lysis of blood cells. In one example implementation taught by International Patent Application No. PCT/CA2013/000992, the blood lysis reagent may be an aqueous liquid including saponin and sodium polyanetholesulfonate (a sodium salt of polyanetholesulfonic acid, known as SPS), and a blood lysis reagent having such a composition is henceforth referred to as a “type 1” blood lysis reagent. The blood lysis reagent may also include an antifoaming agent, such as poly (propylene glycol) (PPG, e.g. with a molecular weight of approximately 2000). International Patent Application No. PCT/CA2013/000992 teaches example concentration ranges of saponin and SPS for a type 1 blood lysis reagent, upon mixing whole blood and the blood lysis reagent, of approximately 1.5 to 80 mg/mL and 0.5 to 20 mg/mL, respectively.

As taught in International Patent Application No. PCT/CA2013/000992, SPS is an anti-coagulant and anti-phagocytosis agent and is known to inhibit antimicrobial agents (Sullivan, N. M., Sutter, V. L., & Finegold, S. M. (1975). Practical aerobic membrane filtration blood culture technique: development of procedure. Journal of clinical microbiology, 1(1), 30-36). The mechanism by which SPS assists in blood cell lysis is not well understood. Without intended to be limited by theory, it is believed that SPS may offer some level of protection to the microorganisms during blood cell lysis, reduce the incidence of entrapment of bacteria in cell debris, and/or reduce the quantity of lysis debris components that may otherwise be present in the sediment.

In another example implementation of a blood lysis reagent composition taught by International Patent Application No. PCT/CA2013/000992, a blood lysis reagent may be an aqueous liquid including Triton X-100 and SPS in a buffer having a pH ranging from 9 to 11, and a blood lysis reagent having such a composition is henceforth referred to as a “type 2” blood lysis reagent. The blood lysis reagent may also include an antifoaming agent, such as poly (propylene glycol) (PPG, e.g. with a molecular weight of approximately 2000). International Patent Application No. PCT/CA2013/000992 teaches example concentration ranges of Triton X-100 and SPS for a type 2 blood lysis reagent, upon mixing whole blood and the blood lysis reagent, of approximately 0.5 to 1.5% w/v and 5 to 10 mg/mL, respectively.

As noted above, the type 1 blood lysis reagent composition described above was found to be suitable for manual and semi-automated separation and concentration of microbial cells from whole blood as per the teachings of International Patent Application No. PCT/CA2013/000992. However, when adapting the reagent formulations disclosed in International Patent Application No. PCT/CA2013/000992 to the automated separation and concentration, and subsequent identification, of microbial cells from whole blood as per the automated methods of International Patent Application No. PCT/CA2015/050449, titled “Apparatus, System and Method for Performing Automated Centrifugal Separation”, filed on May 19, 2015, which is hereby incorporated by reference in its entirety, the present inventors found that the type 1 blood lysis reagent composition was most suitable for cases in which the quantity of whole blood was less than approximately 1 ml.

Another example blood lysis reagent for achieving a low surface density of lysis debris particles is described in International Patent Application No. PCT/CA2019/050716, which describes example blood lysis reagent compositions and method for preserving microbial cell viability and lowering the sample viscosity. This blood lysis reagent composition, henceforth referred to as a type 3 blood lysis reagent, may be provided containing saponin, SPS, an alkaline buffer and optionally a non-ionic surfactant.

In one example embodiment, the type 3 blood lysis reagent may have a composition such that after the blood lysis reagent is mixed with the sample, the concentration of saponin lies between 3 and 60 mg/ml, the concentration of SPS lies between 1.5 and 50 mg/ml, the concentration of non-ionic surfactant lies within 0-3% w/v and the pH lies within a range of 7.8-10. In some example embodiments, the buffer concentration may be selected such that the effective buffer concentration lies in the range of 10-300 mM. It will be understood that a suitable concentration range for a given components of a blood lysis reagent can be determined, for a given set of conditions, by experimentally investigating the effect of changes in concentration of the given component on one or more performance metrics, such as, but not limited to, blood lysis efficiency, quantity of residual blood cell debris, microbial cell intactness and microbial cell viability. A type 3 blood lysis reagent may be provided as two or more reagents that can be stored separately and mixed prior to use, such that the saponin component of the blood lysis reagent is stored in an acidic environment that is separated from the alkaline component of the blood lysis reagent. In one example implementation, one or more of the reagents that are mixed to form the final blood lysis reagent may be stored in a solid phase.

Accordingly, in some example implementations, a sample may be processed by a type 3 lysis reagent comprising saponin, sodium polyanethole sulfonate (SPS), a non-ionic surfactant (such as, but not limited to, Triton™ X-100), a buffer (e.g. a carbonate-bicarbonate buffer), with an alkaline pH. The blood lysis reagent may also include an antifoaming agent such as SE-15 (e.g. a 10% emulsion w/v of active silicone polymer and non-ionic emulsifiers).

A blood lysis reagent without a non-ionic surfactant and a carbonate-bicarbonate buffer is known to be benign to microbial cells. However, as illustrated below, in the case of processing whole blood samples, the size of the blood debris transferred to the final cell suspension according to the use of such a blood lysis reagent has been found to give rise to an elevated level of surface artefacts (background) that can impede microcolony detection and characterization. In contrast, addition of moderate amounts of non-ionic surfactant (e.g. Triton™M X-100) and carbonate-bicarbonate buffer, along with saponin and SPS, while not significantly impacting the cell viability, can be beneficial in significantly reducing the surface artefacts arising from lysis debris.

In some example implementations, a whole blood sample with a volume up to 10 mL may be mixed with a type-3 blood lysis reagent such that the concentration of saponin in the final mixture ranges between 10-30 mg/ml (or, in some example implementations, 3-60 mg/ml), the concentration of SPS in the final mixture may range between 5-50 mg/ml (or, in some example implementations, 1.5-50 mg/ml), the effective buffer concentration lies in the range of 10-300 mM, the concentration of non-ionic surfactant lies within 0-3% w/v (or, in some example implementations, 0-1% w/v), the pH of the final mixture may range between 7.8-10 (or, in some example implementations, 8.2-9.5), and the concentration of the antifoaming agent emulsion lies within 0.005 to 0.5% (v/w).

The results shown in the examples below suggest that the size of the debris particles is strongly influenced by the composition of the blood lysis reagent when processing blood samples for subsequent direct colony growth. However, the requirement for the microbial cell viability, particularly in the case of Gram-negative bacteria, limits the ability to achieve smaller debris size by increasing the digestion capacity of the blood lysis reagent. This limitation can be tolerated as long as the sizes and the density of the debris does not impede the detection of microbial cell growth at microcolony level. One example criterion in this respect is that after spreading the final cell suspension on the solid growth medium, the lysis debris artefacts should not cover the surface of the solid growth medium with an areal fraction exceeding 90%, or preferably not exceeding 50%, or more preferably not exceeding 20%. The present inventors have found that this criterion can be satisfied by processing whole blood samples with a type-3 blood lysis reagent.

Provided that the surface density of artefacts is not prohibitively high, it is feasible to differentiate the background from microcolonies by recording multiple pictures over a time period via time-lapse imaging, or, for example, removing the background by size thresholding, for example, as described above. In order to perform size selection, an image of the gel surface can be taken from a limited area of the surface before or slightly after incubation. In one example implementation, image analysis is performed to determine the average size of the debris particles, R_{back.av}, and the standard deviation of the debris particle size, sd, and a size threshold may be determined based on R_{back.av} and sd. The size selection criterion can be set, for example, as R>R_{threshold}=R_{back.av}+n*sd, where n is typically an integer selected in the range 1 to 6, and more typically n=3. In another embodiment, R_{back.av} and sd may be predetermined (e.g. and provided along with the cartridge or embedded in software of the device/instrument) to become available at the colony analysis stage.

In some example embodiments, the area over which the microbial suspension, after lysis of host blood cells and separation from a whole blood sample is inoculated, maybe be selected to be between 50-200 mm² per 1 ml of initial whole blood sample, in order to avoid formation of a continuous layer of debris and to facilitate the early detection of microbial cells. In some example implementations, the area over which the concentration microbial suspension, after lysis of host blood cells and separation from a whole blood sample, maybe be selected to be between 25-50 mm² per 1 ml of initial whole blood sample, or between 50-100 mm² per 1 ml of initial whole blood sample, or between 50 -150 mm² per 1 ml of initial whole blood sample, or between 50-300 mm² per 1 ml of initial whole blood sample.

In some example embodiments, conventional culture bottles may be employed for sample collection, and the culture bottles may be processed, optionally in the absence of initial incubation, to separate the microbial cells in the culture bottles and to provide a concentrated suspension of viable microbial cells for inoculation onto a solid growth medium. Blood culture bottles typically either include resins for absorbing antimicrobials or rely on partial antimicrobial inactivation by included commands such as sodium polyanethole sulfonate (SPS) and saponin which are common additives to lytic bottles. In addition, the potency of antimicrobials is reduced by ~5-fold dilution (the ratio of culture media to blood). The drawback with the bottle as a collection device is 1) the requirement for approaches to isolate cells from large volumes of about 40 mL, which are often cumbersome and difficult to automate, 2) issues with resin beads, media particles during separation, and 3) blood coagulation which impacts the cleanness of final cell suspension.

The preceding examples of blood lysis reagents and separation methods (centrifugation, filtration, microfluidic) have demonstrated various approaches to obtaining a separated suspension of viable microbial cells. These methods, and other methods known in the art, may be adapted by the skilled artisan, in combination with the selection of a suitable initial quantity of sample, in order to achieve a debris field density after inoculation on the solid phase medium that is sufficient for microcolony detection according to a desired temporal detection window.

The present inventors have found that in order to achieve reliable microcolony detection within 2-3 hours, with microcolony detection typically occurring for a microcolony diameter in the range of 20-70 μm, 20-60 μm, or 20-50 μm, a desired post-inoculation artefact surface coverage lies in the range of less than 80%, less than 70%, less than 60%, less than 50%, less than 40%, less than 30%, or less than 20%, and in some example embodiments from 10-80% 10-70%, 10-60%, 10-50%, 10-40%, 10-30%, or 15-30%. The present inventors have also found that in some cases, it may be beneficial to select the lysis reagent and separation method such that the mean debris particle size, in terms of an average lateral dimension measured within a plane parallel to the solid growth surface, is, for example, less than 10 microns, or less than 8 microns, or less than 6 microns, or, for example, 1-10 microns, 1-8 microns, or 1-6 microns.

### Parallel Incubation of Other Detected Microcolonies

In some example implementations, a second detected microcolony may be further incubated, in parallel with performing nucleic acid amplification and/or sequencing based on microbial cells harvested from a first detected microcolony, and the cells from the second detected microcolony may be subsequently harvested after it has been determined to include a minimum number of cells for performing a downstream application, such as, for example, microbial cell identification by MALDI (e.g. >10⁵ cells). In some example implementations, a third detected microcolony may be further incubated, in parallel with performing nucleic acid amplification and/or sequencing based on microbial cells harvested from a first detected microcolony, and the cells from the third detected microcolony may be subsequently harvested after it has been determined to include a minimum number of cells for performing another downstream application, such as, for example, antimicrobial susceptibility testing. The identification results, known species-specific breakpoints, and other clinical factors may be employed when selecting an antibiotic and dose based on the AST results.

As noted above, in some example embodiments, AST may be performed on microbial cells harvested from one or more colonies, optionally after having performed at least a presumptive identification or classification of the colony microbial cell class (in some cases, presumptive identification may not be necessary, for example, if a colony is harvested after reaching a size known to have a minimum cell count across a wide variety of microbial cell classes, and if a broad AST panel is employed, for example, a panel that is sufficiently broad for to provide coverage for sets of both Gram positive and Gram negative bacteria). The harvested colonies (e.g. obtained using manual harvesting, automated harvesting, or a combination thereof) may then be suspended in a liquid to form a suspension (optionally diluted or concentrated), aliquoted and contacted with different concentrations of antibiotics. The antibiotics (and optionally concentrations thereof) may be selected based on the identity of the microbial cells. For example, the aliquoted microbial cells may be contacted with three different concentrations of the selected antibiotics and microbial cell growth may be subsequently monitored to determine a measure of susceptibility and/or resistance. In other example embodiments, additional concentrations of antibiotics may be employed.

### Automated Workflows

As noted above, the methods disclosed herein may be performed using a manual, automated, or semi-automated workflow. In some example implementations, all of the method steps involved in initial microbial cell separation, solid growth medium inoculation, solid growth medium incubation and microcolony monitoring, microcolony harvesting, preparation of a microbial suspension from the harvested microbial cells, optional lysis of the harvested microbial cells, and performing nucleic acid amplification and/or sequencing based on the harvested microbial cells, may be automated in a single instrument. In other example embodiments, two or more instruments may be employed, with each instrument performing one or more of the aforementioned steps. For example, a first instrument may perform lysis of host cells and separation of microbial cells, such as the automated cartridge-based centrifugation device disclosed in International Patent Application No. PCT/CA2015/050449, titled “APPARATUS, SYSTEM AND METHOD FOR PERFORMING AUTOMATED CENTRIFUGAL SEPARATION”, which is incorporated herein by reference in its entirety. In some example embodiments, the inoculation step may be integrated with the separation step in a closed cartridge, for example, as disclosed in International Patent No. PCT/CA2019/051895.

### Time and Speed

Unlike prior methods of colony growth and subsequent analysis, various example embodiments of the present disclosure facilitate the rapid detection of an infection by detection of the presence of microcolonies based on the inoculation of a solid growth medium in the absence of a preceding liquid-phase culture step, and the rapid detection of nucleic acids, via amplification and/or sequencing, within hours of initial inoculation of the sample onto the solid growth medium.

In some example implementations, a microcolony may be harvested for subsequent nucleic acid amplification within 1.5 to 2.5 hours, 2 to 3 hours, 2 to 4 hours, and 2 to 5 hours of initiating processing of a sample.

In some example implementations, nucleic acid amplification and/or sequencing may be initiated within 2 to 3 hours, 2 to 4 hours, 2 to 5 hours and 2 to 6 hours of initiating processing of a sample.

In some example implementations, nucleic acid amplification results may be obtained within 3 to 4 hours, 3 to 5 hours and 3 to 6 hours of initiating processing of a sample.

In some example implementations, a microcolony may be harvested for subsequent nucleic acid amplification within 5, 10, 15, 20, 25, 30, 35, 40, 45, 50, 55 or 60 minutes of initial microcolony detection.

In some example embodiments, nucleic acid amplification and/or sequencing based on microbial cells harvested from a microcolony may be initiated within 5, 10, 15, 20, 25, 30, 35, 40, 45, 50, 55 or 60 minutes of harvesting the microbial cells.

In some example embodiments, nucleic acid amplification and/or sequencing based on microbial cells harvested from a microcolony may be initiated within 5, 10, 15, 20, 25, 30, 35, 40, 45, 50, 55 or 60 minutes of performing lysis of microbial cells harvested from a microcolony.

### Lysis of Microbial Cells

As described above, the harvested microbial cells are lysed prior to performing the nucleic acid amplification reaction and/or sequencing. As described in detail below, the present inventors have found that a wide variety of lysis methods may be employed to successfully detect nucleic acids from harvested microcolonies. Non-limiting example methods of lysis include bead-beating, ultrasonic lysis, heat lysis, and electrical lysis.

The present inventors have found the surprising result that heat lysis may can be a sufficient lysis method for a broad range of Gram negative and Gram positive pathogens, unlike the conventional expectation in the art that heat lysis is typically insufficient for the lysis of Gram positive microbial cells.

In other example embodiments, lysis maybe be performed electrically, for example, according to the methods disclosed in International Patent Application No. PCT/CA2012/000698, titled “METHODS AND DEVICES FOR ELECTRICAL SAMPLE PREPARATION”, which is incorporated herein by reference in its entirety. For example, a suspension of microbial cells obtained by resuspending the harvested microbial cells in a liquid (having an ionic strength between 0.1 mM and 10 mM) may be electrically processed, within a fluidic channel, by applying bipolar voltage pulses between an upper electrode and a lower electrode such that an electric field generated across the thickness of the fluidic channel is between approximately 2 kV/cm and 30 kV/cm, and such that the liquid is heated with a heating rate of at least 250 degrees per second.

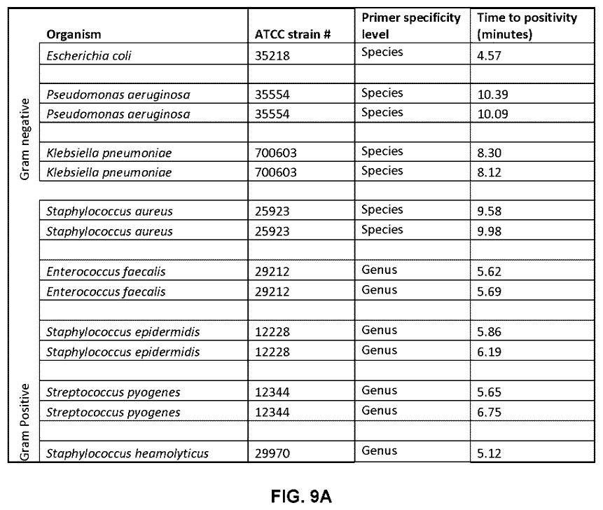

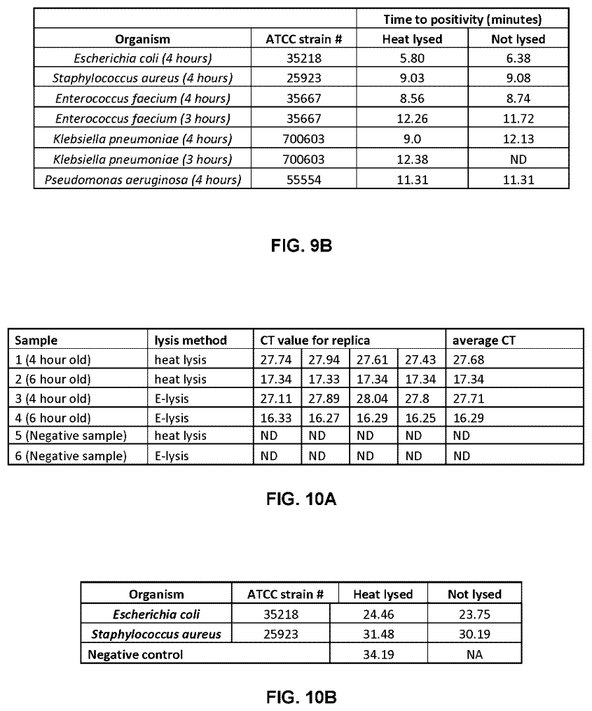

To demonstrate this efficiency of lysis from cells harvested from a microcolony, and as described in the examples provided below, the present inventors performed heat lysis followed by real-time reverse transcriptase Loop-mediated isothermal amplification (rRT-LAMP). rRT-LAMP is a molecular diagnostic technique that sensitively detects RNA templates under isothermal conditions, typically at 60 to 65° C. For the present experiments, various 6-primer sets were designed for detecting bacterial cells bacterial species with high specificity either to species or Genus level (as indicated in the FIG. 9A) via transcribing and amplifying ribosomal RNA released via heat lysis. As it is known in the prior art, heat lysis, while being simple and easy to use, is very inefficient for releasing ribosomes from Gram-positive bacteria.

The experiment was performed according to the method of Example 9 and the result was presented in FIG. 9A. The assay performance in this case is determined by “Time To Positivity (TTP)”. The shorter TTPs are indicative of robust assay as times and TTP>15 minutes is judged to be false positive detection arising from the non-specific amplification of contaminations. In contrast to the case of Gram-negative bacteria, poor performance was expected for Gram positive bacteria as the stronger cell walls of mature Gram-positive bacteria prevent the release of ribosomes (the source of rRNA templates). This unexpected observation highlights one advantage of using cells harvested from microcolonies for molecular assay considering that heat lysis is easier to implement in cartridges. More surprising results are presented in FIG. 9B, which compares the performance of RT-LAMP assay for two cases: 1) cell suspension subjected to heat lysis, and 2) no cell lysis was performed prior to RT-LAMP. As it is observed, the assay performances are similar except in the case of Klebsiella pneumoniae. Interestingly, even in this case, the assay on cell suspension obtained from 4 hour-old microcolony gave acceptable performance.

The advantage of harvesting cells from microcolonies, in terms of ease of cell lysis, was further illustrated by performing PCR detection of rRNA gene on genomic DNA (gDNA). For this end, 2-primer sets were designed for detecting E. coli or S. aureus bacterial species with high specificity. The experiment was performed according to the method of Example 10. In the experience of the present inventors, the assay performance is considered to be acceptable if the CT value is less than 35 cycles. Of course, occasional contaminations by templated from environment may give rise to false positive values. However, these occasional cases have CT values of above 32 cycles and lower CT values correspond to robust detection.

The CT values corresponding to the PCR detection of E. coli or S. aureus bacterial species, obtained from 4 or 6 hours old microcolonies, are presented in FIG. 10A. In both cases of employing heat lysis and electrical lysis methods the assay performance is excellent.

In another experiment, the possibility of performing robust PCR assay on cell suspensions obtained from microcolonies in the absence of the cell lysis step was investigated. The result, which is presented in FIG. 10B, surprisingly indicate that the performance is similar to the case of employing heat lysis method. These observations highlight an advantage of obtaining cells from microcolonies in that in some cases, the cell lysis may be avoided, or, for example, limited to heat or electrical lysis methods which appear to achieve suitable lysis even in the case of Gram positive bacterial cells.

In contrast to glass bead beating, lysis methods such as heat lysis and electrical lysis may be beneficial in preserving the integrity gDNA released via lysis. Accordingly, heat lysis and/or electrical lysis may be beneficial sequencing applications in which longer nucleic acid fragments are desirable. On the other hand, glass bead beating may be beneficial as a lysis method for bacterial identification via PCR amplification of specific region on the gDNA, because bead beating shears gDNA and may therefore facilitate amplification of multiple copies of the gene (~10 in the case of rRNA gene). However, this latter aspect may be less important when obtaining cells from microcolonies due to the high number of cells.

### Examples of Types of Solid Growth Media

It will be understood that a wide variety of solid growth media may be employed to facilitate the growth and detection of microcolonies. The solid growth medium is suitable for providing the microbial cells with an appropriate source of growth media to support colony formation during incubation. Non-limiting examples of solid growth media include conventional agar, gelatin, guar gum, xanthan gum, having suitable growth nutrients. In some example implementations, the solid growth media may be chromogenic according to the type of microbial cell. In some embodiments chromogenic or fluorogenic substrate may be added to the agar media for identifying the microorganism by specific or non-specific staining of the colonies.

The solid growth media may be in a dry or partially dry format such that that a liquid component from the cell suspension is absorbed (at least partially) upon contact.

In some example embodiments in which the solid growth medium comprises agar, the agar concentration may lie within 1.5% to 2.0%, which can exhibit a substantially harder surface than gels formed from lower agar concentrations. The present inventors have found that such harder surfaces are less prone to surface damage during microcolony harvesting. This aspect is advantageous when the microcolony is desired to keep growing for subsequent harvesting episodes. In addition, the likelihood of transferring portions (pieces) of gel to the resulting cell suspension is substantially lowered. Such gel portions, if not removed by methods such as filtering, may deteriorate the performance of the downstream assay. In one embodiment, as taught in Example 3, the gel composition is optimized for the growth of fastidious organisms.

### Rapid Gram Stain from Microbial Cells Harvested from Microcolonies

While the preceding example embodiments have disclosed methods in which microbial cells harvested from a microcolony are employed to rapidly perform nucleic acid amplification or sequencing of nucleic acids, the present inventors have also found that microbial cells harvested from a microcolony may be directly employed for Gram Staining. Accordingly, such a microcolony-based Gram stain method enables, with mere hours of the initiation of sample processing, both the detection of an infection via rapid microcolony detection, and (ii) the determination of the Gram status of the detected microcolonies. In some example implementations, a rapid, microcolony-based Gram stain may be performed on one detected microcolony soon or immediately after microcolony detection, and another microcolony may be separately processed for AST, with the rapid microcolony-based Gram stain result being employed to select an appropriate subset of antimicrobial agents for AST.

Experimental results demonstrating the ease and clarity of microcolony-based rapid Gram staining are presented in Example 1A below.

### Improved Methods and Blood Lysis Reagent for Processing Whole Blood Samples

As described above, many attempts have been made to facilitate the separation of microbial cells from whole blood, in order to enable subsequent processing that avoids the long delays associated with liquid blood culture.

For example, the Isolator®, developed in the 1970s and refined in the 1980s by Dorn, is a sample collection and transport vessel that houses a blood lysis reagent containing saponin and sodium polyanethole sulfonate (SPS). After collecting a whole blood sample into the vessel, where the whole blood sample is mixed with the blood lysis reagent, the vessel is transported to a laboratory, where it is subsequently centrifugally processed to provide a concentrated microbial cell suspension that is plated onto a solid growth medium. Colonies are typically detectable on the growth medium approximately 24 to 48 hours after inoculation.

Although the Isolator™ avoids the need for liquid blood culture, it suffers from the same long detection time that is associated with liquid blood culture due to the delays prior to the detection of macroscopic colonies. The present inventors realized that this drawback of the Isolator™ is associated with insufficient digestion and removal of residual blood debris and set out to develop improved blood lysis reagent formulations and methods that would facilitate the detection of microbial cells within minutes or hours of separation of whole blood. The present inventors began by seeking improvements to Dorn's method that was based on a blood lysis reagent composition including saponin and SPS.

As noted above, International Patent No. PCT/CA2019/050716, titled “METHODS AND COMPOSITIONS FOR THE SELECTIVE LYSIS OF BLOOD CELLS AND SEPARATION OF MICROBIAL CELLS” discloses improved blood lysis reagents and microbial separation methods were disclosed that combined the hemolytic benefits of saponin, the microbial protection and anticoagulation benefits of SPS, and the enhanced blood debris digestion capability and lower viscosity provided by an alkaline environment, optionally with an increased saponin concentration relative to that of the Isolator™ to further enhance blood cell lysis and digestion.

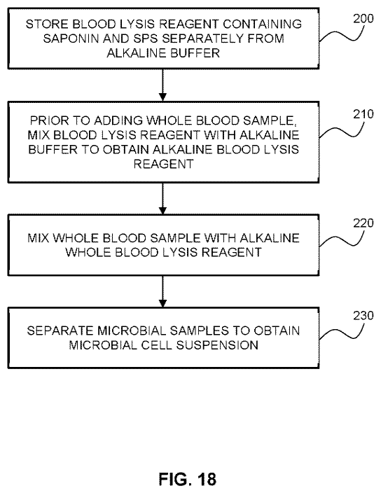

FIG. 18 summarizes a blood lysis workflow described in this patent application. A blood lysis reagent containing saponin and SPS is stored separately from an alkaline buffer, as shown at 200. Prior to adding a whole blood sample, the blood lysis reagent is pre-mixed with the alkaline buffer in order to form an alkaline blood lysis reagent that includes saponin and SPS, as shown at 210. The whole blood sample is then mixed with the alkaline blood lysis reagent to form a mixture, as shown in step 220. A separation process, such as centrifugation, filtration, or microfluidic separation, is then employed at step 230 to separate microbial cells from the mixture and obtain a microbial cell suspension at step 230.

This blood lysis formulation and method illustrated in FIG. 18 was shown to enable improved digestion of residual blood debris and to facilitate the direct molecular processing of separated microbial cells. Moreover, as disclosed in PCT Patent Application No. PCT/CA2019/051895, titled “SYSTEMS AND METHODS FOR MICROCOLONY GROWTH AND MICROBIAL CELL CHARACTERIZATION”, the whole blood lysis and separation method illustrated in FIG. 18 was shown to enable microcolony detection within mere hours of inoculation for a wide variety of bacterial and fungal species, in stark contrast to the days required for colony detection after inoculation of the concentrate obtained from the Isolator™.

While the alkaline saponin and SPS-based blood lysis reagent disclosed in PCT/CA2019/050716 was found to be particularly amenable to the processing of whole blood samples with volumes in the 1-3 ml range, the present inventors found that the processing of whole blood samples with larger volumes could result in higher amounts of residual blood lysis debris, which could lead to performance limitations in some downstream applications. For example, the present inventors found that residual blood debris arising from larger sample volumes could impair the ability to perform rapid molecular detection of separated microbial cells and/or impair the rapid detection of microcolonies grown from a separated microbial cell suspension. It was also found that for such large volume whole blood samples, the residual blood debris could result in the entrapment of microbial cells during the separation process, leading to a loss in recovery that can be of significant consequence for low-titre whole blood samples that are known to exhibit microbial cell counts in the 0.1-10 CFU/ml range.

The present inventors set out to improve on the blood lysis reagent and microbial cell separation methods taught in PCT/CA2019/050716 with the goal of developing an improved workflow that would facilitate the processing of blood samples with sample volumes in the range of 5-10 ml (or larger volumes ranges, such as 5-15 ml or 5-20 ml) with greater efficiency in the digestion and removal of residual blood debris. It was hypothesized that improved digestion and removal of blood debris could be achieved by modifying the method of FIG. 18 so that the saponin and SPS-containing blood lysis reagent is initially contacted and mixed with the whole blood sample prior to the addition of the alkaline buffer, instead of pre-mixing the saponin and SPS-containing blood lysis reagent with an alkaline buffer prior to addition of the whole blood sample.

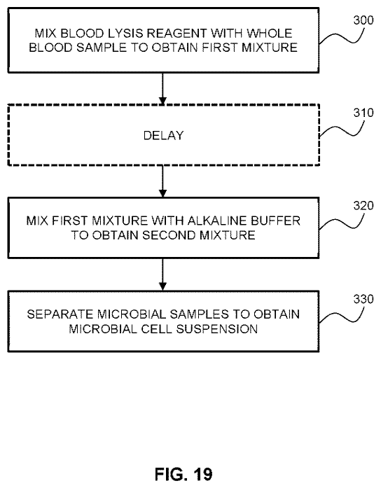

This modified method is illustrated in FIG. 19, which, unlike the method of FIG. 18, begins with the mixing of a whole blood sample with a blood lysis reagent that contains saponin and SPS, as shown at step 300, prior to contact with the alkaline buffer. As shown in step 320, after having formed the first mixture, the first mixture is mixed with the alkaline buffer to form a second mixture. The alkaline buffer is selected with a pH and ionic strength such that the second mixture is alkaline.

As shown at step 330, a separation process is then employed to separate the microbial cells from the second mixture and provide a purified microbial cell suspension. Non-limiting examples of suitable separation methods include centrifugation, with one or more optional washing and centrifugation steps, filtration, microfluidic separation and immunomagnetic separation. An example automated system for performing microbial cell separation and concentration is described in International Patent Application No. PCT/CA2015/050449.

The separated microbial cells, e.g. in the form of a microbial suspension as shown in FIG. 19, may then be processed according to a wide variety of growth and/or analytic steps, examples of which are described below.

Without intending to be limited by theory, the present inventors hypothesized that the modified approach shown in FIG. 19, in which the blood lysis reagent and the alkaline buffer are serially added, instead of being-premixed prior to contact with the whole blood sample, and in which the whole blood sample is initially contacted with the blood lysis reagent in prior to addition of the alkaline buffer, would facilitate more efficient lysis and digestion of blood cells during the initial contact phase with the blood lysis reagent. Experiments validating this hypothesis are presented below.

Moreover, it was hypothesized that in cases in which a time delay is incurred prior to addition of the alkaline buffer and separation of the microbial cells, such as in example workflows in which the first mixture is initially formed when or soon after the whole blood sample is collected from a patient, and is then transported to another location (such as a laboratory or central sample processing facility), the higher viscosity of the first mixture, relative to the lower viscosity that occurs after addition of the alkaline buffer, would provide at least some degree of protection from antimicrobial agents that may be present in the whole blood sample, thus preserving the viability of microbial cells during sample transport.

As shown in FIG. 19, the modified serial workflow may include a time delay between (i) the mixing of the whole blood sample and the blood lysis reagent to form the first mixture and (ii) the addition of the alkaline buffer. This time delay may be incurred, at least in part, during transport of the first mixture from a blood collection location to a sample processing location.

In some example implementations, the blood lysis reagent may be stored in a partially evacuated vessel, such as a partially evacuated tube, with a pressure that is sufficiently low to facilitate collection of a whole blood sample of a desired volume. The mixing of the whole blood sample with the blood lysis reagent results in a first mixture within which blood cells are lysed and blood cell debris is digested, at least in part. The vessel may be transported to a separate processing location, during which the delay 310 shown in FIG. 19 may be incurred, at least in part.

In some example implementations, the alkaline buffer may be stored in a chamber within a fluidic cartridge that is configured to be actuated to perform the separation of the microbial cells. The first mixture may be added to the fluidic cartridge to mix the first the mixture with the alkaline buffer, in a manual, automated or semi-automated manner. Non-limiting examples of automated instruments and fluidic cartridges that employ centrifugation for microbial cell separation are disclosed in International Patent Application No. PCT/CA2015/050449, titled “APPARATUS, SYSTEM AND METHOD FOR PERFORMING AUTOMATED CENTRIFUGAL SEPARATION”.

In some example implementations, a fluidic cartridge may be provided that houses both the blood lysis reagent and the alkaline buffer, where the blood lysis reagent is housed in a first chamber within the fluidic cartridge and the alkaline buffer is stored in a second fluidic chamber within the fluidic cartridge, where the first and second chambers are initially absent of fluidic communication, e.g. via one or more valves that are provided in a (passively) closed state. The first chamber, containing the blood lysis reagent, may be provided in a partially evacuated state, with a pierceable and re-sealable top (e.g. stopper), such as a rubber, elastomeric or other suitable material, and the first chamber may have an initial pressure that is sufficiently low to facilitate passive collection of a whole blood sample of a desired volume to form the first mixture. The cartridge may then be transported to a location where the automated instrument resides, with the one or more valves closed during transport to prevent fluidic contact of the first mixture with the alkaline buffer, with the delay 310 in FIG. 19 being incurred during transport to facilitate blood cell lysis and digestion of blood debris. The fluidic cartridge may then be processed by the automated instrument to open the one or more valves, mix the first mixture with the alkaline buffer to obtain the second mixture, according to the method of FIG. 19, and perform one or more additional automated steps to separate the microbial cells from the second mixture (e.g. via centrifugation, filtration, microfluidic separation, and immunomagnetic separation).

The present inventors performed experiment to study the impact of the time delay in step 310 of FIG. 19 (the delay between mixing a whole blood sample with a blood lysis reagent that contains saponin and SPS to form a first mixture, and subsequently mixing the first mixture with an alkaline buffer to form a second mixture) on the resulting blood lysis efficiency and digestion of blood debris.

A large 7 mL sample volume of whole blood was collected into an evacuated tube containing 2 mL of a blood lysis reagent including saponin and SPS. The blood lysis reagent had the following composition (prior to sample addition): Tergitol™ L-62 (25 mM or 50 mM) 0.20% w/w, methyl-beta-cyclodextrin (MBCD) 6.25 mM, SPS 4.25% w/w, triterpenoid saponin 5.00% w/w, acetate buffer 150 mM. The tube was inverted several times to mix the blood lysis reagent with the whole blood sample, forming the first mixture, as per the method shown in FIG. 19. Subsequently, a 20 μL aliquot was removed every 10 minutes for microscopy imaging and viscosity measurements. The alkaline buffer (0.15 M Tris (Tris(hydroxymethyl)aminomethane) buffer) was added after 40 minutes. Microscopic imaging of the mixture was performed according to Wright's staining, as described in Example 16.

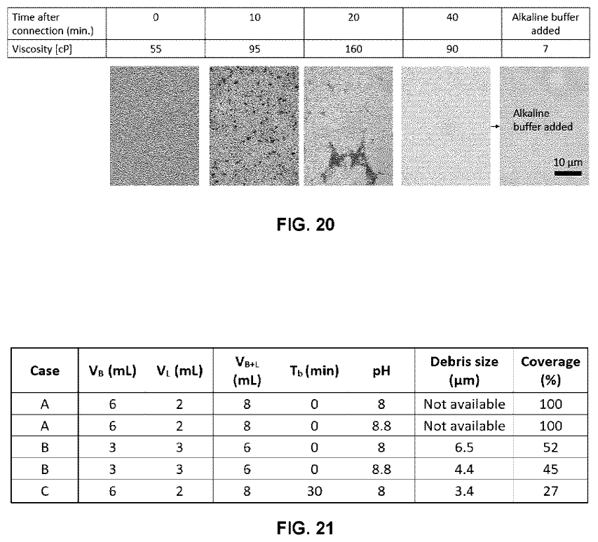

The resulting microscopy images are shown in FIG. 20, revealing the time dependence of the lysis of blood cells and the digestion of blood debris by the blood lysis reagent. At t=0, the smear on the microscopy slide is too thick to reveal internal morphology and the image is absent of characteristic features. At 10 minutes, most of the red blood cells have been lysed, with only platelets and white blood cells being observed. After 20 minutes, the platelets and white blood cells are no longer distinguishable and only extended filamentary structures are observable. After 40 minutes, the filamentary structures are no longer observable.

The time dependence of the viscosity, after formation of the first mixture, and the reduced viscosity after addition of the alkaline buffer, was estimated by measuring of travel distance of a 10 μL drop on a plastic sheet and comparing it with the travel distances of droplets of succories solutions (succors viscosity reference) on the same sheet. As can be seen in FIG. 20, the viscosity was observed to increase over the first 20 minutes and then slowly drops to a value of ~90 cP after 40 minutes, prior to addition of the alkaline buffer.

After 40 minutes, the alkaline buffer was mixed with the first mixture, thereby obtaining the second mixture that had a basic pH. The addition of the alkaline buffer increased the pH of the mixture to 8.5 and significantly reduced the viscosity from 90 cP to 7 cP. This reduction in viscosity, which is beneficial in facilitating efficient microbial cell separation and debris removal by filtering, centrifugation or other separation methods (e.g. microfluidic separation), has been observed to occur independent of the time delay between contacting the whole blood sample with the blood lysis reagent to form the first mixture, and subsequently contacting the first mixture with the alkaline buffer to form the second mixture.

Although FIG. 20 shows that the filamentary structure is absent in the microscopy image taken after addition of the alkaline buffer, the present inventors have found that such filamentary structures can persist, even after the alkaline buffer is added, if there has been an insufficient time delay between the formation of the first mixture (mixing the blood lysis reagent with the whole blood sample) and the addition of the alkaline buffer. In cases in which an insufficient time delay has been incurred prior to addition of the alkaline buffer, such filamentary structures can be observed to be floating inside the liquid.

Without intended to be limited by theory, it is suspected that such residual filamentary structures, when present due to incomplete digestion, can trap microbial cells. Such residual filamentary structures can thus impede the separation process due to clogging and reduce microbial cell recovery due to microbial cell loss during separation (e.g. centrifugation or filtration). Such microbial cell loss (recovery loss) can be particularly detrimental when the microbial cell concentration in the original sample is within the 0.1-10 CFU/mL range.

According, in some example embodiments, a delay, shown in FIG. 19 at 310, is provided between the formation of the first mixture, that is, the time of initiating hemolysis (by mixing the whole blood sample with the blood lysis reagent) and contacting (mixing) the first mixture with the alkaline buffer to obtain the second mixture, that is, the time of initiating basification. In some example implementations, the delay between forming the first mixture and contacting the first mixture with the alkaline buffer to form the second mixture is between 15 minutes and 30 minutes, or between 15 minutes and 1 hour, between 15 minutes and 2 hours, or between 15 minutes and 3 hours, or between 15 minutes and 4 hours, or between 15 minutes and 5 hours, or between 15 minutes and 6 hours, or between 15 minutes and 8 hours, or between 15 minutes and 10 hours, or between 15 minutes and 12 hours, or between 15 minutes and 14 hours, or between 15 minutes and 16 hours, or between 15 minutes and 20 hours, or between 15 minutes and 22 hours, or between 15 minutes and 24 hours. In some example implementations, the delay between forming the first mixture and contacting the first mixture with the alkaline buffer is between 30 minutes and 1 hour, between 30 minutes and 2 hours, or between 30 minutes and 3 hours, or between 30 minutes and 4 hours, or between 30 minutes and 5 hours, or between 30 minutes and 6 hours, or between 30 minutes and 8 hours, or between 30 minutes and 10 hours, or between 30 minutes and 12 hours, or between 30 minutes and 14 hours, or between 30 minutes and 16 hours, or between 30 minutes and 20 hours, or between 30 minutes and 22 hours, or between 30 minutes and 24 hours. In some example implementations, the delay between forming the first mixture and contacting the first mixture with the alkaline buffer is between 40 minutes and 1 hour, between 40 minutes and 2 hours, or between 40 minutes and 3 hours, or between 40 minutes and 4 hours, or between 40 minutes and 5 hours, or between 40 minutes and 6 hours, or between 40 minutes and 8 hours, or between 40 minutes and 10 hours, or between 40 minutes and 12 hours, or between 40 minutes and 14 hours, or between 40 minutes and 16 hours, or between 40 minutes and 20 hours, or between 40 minutes and 22 hours, or between 40 minutes and 24 hours.

In some example implementations, the delay between forming the first mixture and contacting the first mixture with the alkaline buffer is sufficiently long that after the delay but prior to addition of the alkaline buffer, the first mixture is absent of red blood cells and white blood cells under microscopic evaluation.

In some example implementations, the delay between forming the first mixture and contacting the first mixture with the alkaline buffer is sufficiently long that after the delay but prior to addition of the alkaline buffer, the first mixture is absent of filamentary structures under microscopic evaluation.

In some example implementations, the delay between forming the first mixture and contacting the first mixture with the alkaline buffer is sufficiently long that after contacting the first mixture with the alkaline buffer, separating microbial cells from the second mixture to obtain a suspension of microbial cells, and inoculating the suspension onto a solid growth medium, the areal coverage of residual blood debris on the solid growth medium is between 5% and 50%, between 5% and 40%, between 5% and 30%, or between 5% and 20%.

The present inventors have found that the inclusion of a delay between forming the first mixture and contacting the first mixture with the alkaline buffer can enable the processing of a higher whole blood volume without intolerably increasing residual blood debris, when compared to a workflow that involves pre-mixing of the blood lysis reagent with the alkaline buffer prior to addition of a whole blood sample.

The present inventors have found that the volume of whole blood, V_{B}, is an important parameter governing the purity of the microbial cell suspension obtained after separation. Specifically, it has been found that higher blood volumes, such as blood volumes between 5 ml and 10 ml, can be more susceptible and sensitive to the detrimental impact of poor recovery due to the low microbial load in typical clinical whole blood samples. Accordingly, some aspects of the present disclosure involve methods that are adapted for the processing of such larger blood volumes.

In order to illustrate this beneficial impact of the time delay on the ability to process whole blood samples of larger volumes without resulting in appreciable blood lysis debris, an experiment was performed that investigated three cases, each case employing different volumes of whole blood (V_{B}), the blood lysis reagent (V_{L}), and total volume of lysed and basified blood (V_{L+B}), with only one case involving a time delay T_{b} between hemolysis and basification. In all cases, the blood lysis reagent had the following composition: Tergitol™ L-62 0.20% w/w, MBCD 6.25 mM, SPS 4.25% w/w, saponin 5.00% w/w, acetate buffer 150 mM. The basification was achieved by adding Tris buffer such that the ionic strength of the final solution is either 20 mM or 60 mM, and the pH of the final solution is either 8 or 8.8.

The volumes and times for the three cases were as follows:
Case A: The blood lysis reagent was premixed with the alkaline buffer to form an alkaline blood lysis mixture according to the method of FIG. 18, and the alkaline mixture was mixed with whole blood that was collected in a standard SPS tube; V_{B}=5 mL, V_{L}=3 mL, V_{L+B}=8 mL.
Case B: The blood lysis reagent was premixed with the alkaline buffer to form an alkaline blood lysis mixture according to the method of FIG. 18, and the alkaline mixture was mixed with whole blood that was collected in a standard SPS tube: V_{B}=2.5 mL, V_{L}=3.5 mL, V_{L+B}=6 mL.
Case C: whole blood was collected in an evacuated tube containing the blood lysis reagent and later mixed with the alkaline buffer after a delay T_{b}=30 minutes, according to the method illustrated in FIG. 19; V_{B}=6 mL, V_{L}=2 mL, V_{L+B}=8 mL.

For each case, the basified mixtures were centrifugally separated and washed according to the method of Example 19 and the separated microbial cells were resuspended to form a microbial cell suspension. 100-120 μL of the resulting cell suspension was spread over an agar plate and after 30 minutes was observed under a microscope. The average debris particle size and the areal surface coverage were measured and are presented in FIG. 21.

Case A, which employed the pre-mixing method of FIG. 18 and involved a high sample volume of 6 ml yet suffered from complete coverage of the solid growth medium, preventing colony detection during the early microcolony phase, for final alkaline pH values of both 8 and 8.8.

The results from Case B, which also employed the pre-mixing method of FIG. 18, were also inferior to those of Case A, even though the volume of whole blood employed was half of that which was employed in Case A, with only 3 ml of whole blood being processed in Case B. Indeed, both experimental implementations of Case B (with the final pH of 8 and 8.8) resulted in significantly larger mean blood lysis debris particle sizes and larger areal fractions.

In stark contrast, Case C, which employed the modified serial method shown in FIG. 19 with a time delay of 30 minutes between hemolysis and basification, resulted in low average debris size (less than 4 μm) and a low areal surface coverage fraction of less than 30%, even for a large whole blood sample volume of 6 mm, enabling the detection of microcolonies without significant performance loss in terms of time to positivity.

The blood lysis reagents described herein employ saponin as a lytic agent, and some embodiments may employ a specific type, concentration range, and/or purity of saponin. Saponins are naturally occurring surface-active glycosides produced by plants, lower marine animals and some bacteria. They exhibit surfactant properties and consist of a carbohydrate moiety linked to a hydrophobic aglycone (sapogenin). Saponins are broadly classified as triterpenoids, steroids or steroidal glycoalkaloids, based on the aglycone structure from which they are derived. The diversity in the structure of different saponins arises primarily due to the variability of the aglycone structure, the nature of the side chains and the position of attachment of these moieties on the aglycone.

The membrane activity of both triterpenoid and steroid saponins has been known to be associated with their ability to interact with membrane-bound cholesterol. Once bound to cholesterol, saponins induce changes in the membrane structure that result in permeability. It is believed that at concentrations greater than its critical micelle concentration (CMC), saponin forms aggregates which bind to the cholesterol within the erythrocyte membrane creating large, stable pores ultimately leading to cell hemolysis. It is this selectivity towards cholesterol containing cell membranes that allow saponin to lyse erythrocytes while leaving bacterial cells in the same medium intact and viable.

Triterpenoid saponins can be found in legumes such as alfalfa, chickpeas, broad beans, soybean, lentils, kidney beans, peanuts, sunflower seeds, ginseng roots, horse chestnut, tea leaves, liquorice roots, quillaja bark, spinach leaves, quinoa seeds, tea leaves, sugar beets and other alliums species, whereas steroidal saponins are found in Yucca, tomato seeds, ginseng roots, yam, aubergine, fenugreek seeds, asparagus, capsicum peppers (Oleszek & Oleszek, 2020). A major source of industrial triterpenoid saponins is the bark of the Quillaja saponaria tree, which is tree native to the Andes region. Saponins represent 20-25% of the extractable material from this source (Barr, et al., 1998, Ad Drug Deliv Rev, 32:247-271).

Saponin solutions are often filter purified after dissolution via filtering devices with various types of membranes. The effect of filtration may vary depending on the source of saponin due to various degrees of refinement that are employed by different commercial vendors. For example, non-refined commercial extracts of Quillaja saponins typically contain approximately 20% triterpenoid saponins by weight, while semi-refined extracts of higher purity may contain 75-80% of triterpenoid saponins by weight (San Martin et al., 2000, J. Sci. Food Agric., 80:2063-2068).

Quillaja saponins are generally weakly acidic. For example, Quillaja saponins can exhibit a pH of between 4 to 5, depending on their purity, when dissolved in water at 20% concentration by weight. At a pH greater than 8, the main Quillaja saponin compounds, QS-7, QS-17, QS-18 and QS-21 will undergo alkaline hydrolysis to generate “deacetylated saponins” DS-1 and DS-2. These deacetylated saponins exhibit approximately 10-fold lower hemolytic activity than their parent saponins (D. J. Pillion, J. A. Amsden, C. R. Kensil, J. Recchia “Structure—function relationship among Quillaja saponins serving as excipients for nasal and ocular delivery of insulin.” J of Pharmaceutical Sciences, Vol 85, No. 5 1996, pages 518-524). At such elevated pH levels, the critical micelle concentration of the saponin mixture will also increase by ten-fold from 200 mg/L at pH 6.5 to 2000 mg/L at pH 10 (W-J Chen, L-C Hsiao, K K-Y Chen “Metal desorption from copper(II)/nickel(II)-spiked kaolin as a soil component using plant-derived saponin biosurfactant.” Process Biochemistry, Vol. 43, No 5 2008, pages 488-498).

As noted above, the saponin material employed for use in preparing the blood lysis reagent may be obtained through the aqueous extraction of the milled inner bark or of the wood of Quillaja saponaria Molina. This early-stage extract is composed of less than 20% w/w triterpenic saponins but in solution it is unstable due to the precipitation of protein-polyphenol complexes. To remove some of the polyphenols and proteins, the extract may be treated to increase its purity and concentration. For example, the saponin extract may be treated with polymeric absorbents such albumin, gelatin, vegetable proteins or polyvinylpolypyrrolidone as well as bentonite followed by filtration through diatomaceous earth, increasing the saponin content to between 20 to 26% by dry weight. The hemolytic activity against sheep's blood at this stage is typically between approximately 150 and 400 μg/mL.

To further purify the saponin extract, additional purified steps may be employed, such as purification by diafiltration and ultrafiltration or by tangential flow filtration using membranes with molecular weight cut-offs of between 10 and 75 kilodaltons. Following such additional purification steps and spray drying, the dried saponin reagent can be increased beyond 75%, and often up to and exceeding 85% w/w (e.g. as measured by UPLC). Such additional purification steps also reduce the concentration of impurities such as polyphenols, polysaccharides, and calcium salts.

The refined saponin material employed to prepare the blood lysis reagent may have a purity of 75-96% triterpenic saponins by dry weight (% w/w), or 80-96% triterpenic saponins by dry weight (% w/w), or 85-96% triterpenic saponins by dry weight (% w/w), or 75-90% triterpenic saponins by dry weight (% w/w), or 80-90% triterpenic saponins by dry weight (% w/w), or 85-90% triterpenic saponins by dry weight (% w/w), or 75-85% triterpenic saponins by dry weight (% w/w), or 80-85% triterpenic saponins by dry weight (% w/w). The refined saponin material employed to prepare the blood lysis reagent may contain impurities, such as, for example, polyphenols and tannins (0.5 to 2.0% w/w), polysaccharides (1 to 5% w/w), plant proteins (2 to 5% w/w), calcium salts such as calcium oxalate (0.2 to 1.0% w/w). Refined saponin material containing 85% triterpenic saponins has been measured in a quantitative dose-response assay to have a hemolytic activity (HC50) between 55 and 88 μg/mL against a 4% suspension of purified sheep's red blood cells in 0.9% w/v sodium chloride in phosphate buffer (the dose-response measurements against purified sheep's red blood cells are used as a proxy for its activity against human red blood cells).

In some example implementations, the blood lysis reagent that is employed to treat a whole blood sample may contain an amount of saponin such that after mixing the blood lysis reagent with a whole blood sample to form the first mixture, the concentration of saponin in the first mixture is between 0.75 and 60 mg/ml, or between 3 and 60 mg/ml, or between 5 and 60 mg/ml, or between 10 and 60 mg/ml, or between 15 and 60 mg/ml, or between 20 and 60 mg/ml, or between 30 and 60 mg/ml or between 30 to 50 mg/ml, or between 20 to 40 mg/ml, or between 10 to 30 mg/ml, or between 5 to 30 mg/ml, or between 3 to 25 mg/ml, or between 0.75 to 25 mg/ml.

In some example implementations, the blood lysis reagent that is employed to treat a whole blood sample may contain an amount of SPS such that after mixing the blood lysis reagent with a whole blood sample to form the first mixture, the concentration of SPS in the first mixture is between 0.35 and 50 mg/ml, or between 1 and 50 mg/ml, or between 2 and 50 mg/ml, or between 5 and 50 mg/ml, or between 10 and 50 mg/ml, or between 20 and 50 mg/ml, or between 20 and 30 mg/ml, or between 15 and 30 mg/ml, or between 10 and 30 mg/ml, or between 5 and 30 mg/ml, or between 2 and 30 mg/ml, or between 1 and 30 mg/ml, or between 0.35 and 30 mg/ml.

The blood lysis reagent may be prepared to have a pH between 3.5 and 8, or between 3.5 and 7, or between 3.5 and 6.95, or between 3.5 and 6.5, or between 3.5 and 5.5, or between 4 and 8, or between 4 and 7, or between 4 and 6.95, or between 4 and 6.5, or between 4 and 5.5, or between 4.5 and 8, or between 4.5 and 7, or between 4.5 and 6.95, or between 4.5 and 6.5, or between 4.5 and 5.5. Example buffers for preparing the blood lysis reagent include, but are not limited to, acetate, citrate, ascorbate, benzoate or malate buffers.

In some example implementations, the alkaline buffer may have pH between 7.3 to 10.5, 7.5 to 9.5, 7.6 to 9, and 8 to 9. Example alkaline buffers include, but are not limited to, triethanolamine, N-[tris(hydroxymethyl)methyl]glycine (tricine), 2-amino-2-(hydroxymethyl)-1,3-propanediol (tris base), N,N-bis(2-hydroxyethyl)glycine (bicine), N-(1,1-dimethyl-2-hydroxyethyl)-3-amino-2-hydroxypropanesulfonic acid (AMPSO), 2-(cyclohexylamino)ethanesulfonic acid (CHES), 3-(cyclohexylamino)-2-hydroxy-1-propanesulfonic acid (CAPSO), 3-(cyclohexylamino)-1-propanesulfonic acid (CAPS), and sodium carbonate. The ionic strength of the alkaline buffer may be selected based on the alkaline buffer composition and the downstream application (e.g. whether or not the downstream application requires or would benefit from maintaining microbial cell viability). Example ranges of the ionic strength of the alkaline buffer in the final solution (blood plus blood lysis mixture) include, but are not limited to, 10 to 100 mM, or 20 to 60 mM, or 25 to 40 mM.

A blood lysis reagent may have a volume between 0.5 and 2.0 ml, or between 0.5 and 2.5 ml, or between 0.5 and 3.0 ml, or between 0.5 and 4.0 ml or between 2.0 and 4.0 ml, or between 2.0 and 3.0 ml. In some example embodiments, a blood lysis reagent having a volume within one or more of these ranges may be prepared such that when mixed with a whole blood sample containing 3 ml, at least one of the preceding concentration ranges for saponin and SPS, in the first mixture, are obtained. A blood lysis reagent may have a volume within one or more of these ranges may be prepared such that when mixed with a whole blood sample containing 5 ml, at least one of the preceding concentration ranges for saponin and SPS, in the first mixture, are obtained. A blood lysis reagent may have a volume within one or more of these ranges may be prepared such that when mixed with a whole blood sample containing 10 ml, at least one of the preceding concentration ranges for saponin and SPS, in the first mixture, are obtained.

The present inventors have found that the ability to generate a first mixture that is substantially absent of a precipitate, or contains a precipitate that is of sufficiently low quantity so as to permit a desired performance of a downstream detection sensitivity and/or assay performance, may be dependent on achieving a sufficiently level of purity (sufficiently low concentration) of the blood lysis reagent with regard to one or more impurities in the blood lysis reagent.

For example, after forming the first mixture, high amounts of polyphenols can potentially co-precipitate with other blood proteins such as hemoglobin. Accordingly, in some example implementations, the blood lysis reagent that is employed to treat a whole blood sample may contain an amount of polyphenols such that after mixing the blood lysis reagent with a whole blood sample to form the first mixture, the concentration of polyphenols is between 0.02 to 1.3% w/w or between 0.04 to 0.4% w/w or between 0.1 to 0.3% w/w.

The present inventors have also found that some blood lysis reagent impurities can lead to instability during storage, prior to use, for example, by forming colloidal complexes with polyphenol, polysaccharides and proteins. Indeed, the present inventors have found that for some blood lysis formulations, a calcium weight percentage in the blood lysis reagent (e.g. as measured by atomic absorption spectrometry), relative to dry weight of saponin, is between 0.1% and 1%, or between 0.1% and 0.75%, or between 0.1% and 0.6%, or between 0.1% and 0.5%, or between 0.1% and 0.4%, or between 0.05% and 1%, or between 0.05% and 0.75%, or between 0.05% and 0.6%, or between 0.05% and 0.5%, or between 0.05% and 0.4%, or between 0.25% and 1%, or between 0.25% and 0.75%, or between 0.25% and 0.6%, or between 0.25% and 0.5%, or between 0.25% and 0.4%. In some cases, the present inventors have found that when the calcium content, by weight, relative to dry weight of saponin, exceeds 0.5%, turbidity can be observed within the blood lysis reagent within one week of storage at room temperature.

It has also been found that high amounts of calcium and polyphenol, when present together may precipitate from solution as calcium phenolates, especially at pH levels greater than 9. Accordingly, it may be beneficial to ensure that the total calcium content in the blood lysis reagent satisfies one or more of the aforementioned calcium content criteria, and that the polyphenol concentration in the blood lysis reagent also satisfies one or more of the aforementioned polyphenol criteria, especially if the alkaline buffer is configured to result in a pH, in the second mixture, of 9 or higher, and/or, if the alkaline buffer employs a carbonate/bicarbonate buffer system (which can lead to the precipitation of calcium carbonate).

The present inventors have found that some saponin and SPS concentrations in the blood lysis reagent can impact the stability of the blood lysis reagent, for example, by phase separation, which leads to issues such as increasing turbidity or precipitation of solid particles over time. Indeed, when high concentrations of saponin in the blood lysis reagent, such as between 3.75% and 7.50% w/w (for example, approximately 37.5 to 75 mg/ml in a 2 ml volume of blood lysis reagent volume) are present with high concentrations of sodium polyanethol sulfate (SPS), such as between 2.50% and 4.00% w/w (or approximately 25 and 40 mg/ml in a 2 ml blood lysis reagent volume), a small amount of precipitate (such as, for example, between 0.1 mg and 1 mg in 5 ml solution) can form over a period of one month at room temperature. Without intending to be limited by theory, it is suspected that this precipitation may be due, for example, to an interaction of the SPS with the saponins themselves, or, for example, due to the impurities in the saponin material, such as the polyphenols and polysaccharides, forming an insoluble complex. The present inventors have found that the higher the temperature the lytic reagent is stored, the more rapidly this complex is formed. In a collection vessel or device containing 2 mL of such a saponin and SPS-containing blood lysis reagent, some insoluble material may be formed and could potentially be transferred present even after one or more separation steps (e.g. one or more rounds of centrifugation and sedimentation).

It was discovered that the addition of methyl-beta-cyclodextrin (MBCD) to the blood lysis reagent prevents phase separation and the precipitation during storage when such high concentrations of saponin and SPS are present. For instance, combining MBCD at a final concentration of 6.25 mM in the blood lysis reagent containing up to 7.50% w/w (up to approximately 75 mg/ml in a 2 ml volume) of saponin and up to 4.0% w/w (up to approximately 40 mg/ml in a 2 ml volume) of SPS prevented any visible precipitation for at least one month at 45° C., whereas without the MBCD, a significant amount of precipitation was visible after 2 days at 45° C. It is believed that the MBCD stabilizes the other components in the lytic reagent by encapsulating them and preventing their interaction and the creation of any insoluble complexes.

Without intending to be limited by theory, it is also believed that methyl-β-cyclodextrin (MBCD) can also assist in hemolysis since it has the capability to penetrate sections of the cellular membrane that are more resistant to solubilization by other detergents. From a simple point of view, the detergents function by intercalating into the lipid membrane, rearranging the membrane components to accommodate the increasing concentration of detergent, and then ultimately extracting the lipids and proteins into smaller micellar structures. The different fluid states of the lipid membrane influence the extent to which a detergent can disrupt and solubilize the cellular components. Loosely packed membrane domains made from kinked unsaturated glycerophospholipids and fatty acids give rise to liquid-disordered (L_{d}) states that are highly diffuse and easily penetrable by mild detergents. However, some lipids have a greater tendency to associate with each other to form more tightly packed domains creating liquid-ordered (Lₒ) states. These domains are comprised of more hydrophobic lipids, namely cholesterol and sphingolipids as well as saturated fatty acids and glycerophospholipids, and can resist penetration and extraction by detergents. These detergent-resistant membranes (DRM), which are believed to give rise to lipid raft domains, are not completely soluble in most detergents leaving behind insoluble fragments of sphingolipids and cholesterol as well as membrane proteins that remain imbedded in these fragments. The consequence of these DRMs in human red blood cells is incomplete hemolysis by the blood lysing reagent and a heterogeneous mixture of solubilized proteins and lipids with insoluble cellular debris.

Disruption of the DRM can be achieved by extraction of the cholesterol from within the lipid bilayer. Quillaja saponin is known to solubilize cholesterol by incorporating it within the saponin micelle. During hemolysis, it is believed that the saponin undergoes micellar rearrangement with cholesterol in the cellular membrane resulting in the formation of open pores that leads to rupture of the entire cell. β-cyclodextrin (β-CD) also has a high affinity for cholesterol and has been used for extracting cholesterol from erythrocytes and other membranes. The mechanism involves the association of a CD dimer onto the membrane surface followed by extraction of the cholesterol into the hydrophobic cavity of the dimer. The cholesterol-CD complex then desorbs from the membrane surface into the surrounding solution where it can transfer the cholesterol to either another domain of the cellular membrane or potentially to a waiting detergent micelle which can carry it away from the cell. The loss of most of the cholesterol from the membrane destabilizes the DRM, making it more susceptible to solubilization by other mild detergents such as saponin. The result is a more complete hemolysis without interfering solid debris.

While the present inventors have demonstrated the beneficial inclusion of MBCD for reducing or eliminating precipitation during storage of a saponin and SPS-containing blood lysis reagent, in some implementations other β-cyclodextrins may be employed in addition or in the alternative. The skilled artisan will be able to determine a suitable concentration of one or more β-cyclodextrins by performing experiments with different concentrations and observing the presence or absence of precipitation in the blood lysis reagent during storage.

Accordingly, in some example implementations, a blood lysis reagent containing saponin and SPS may also contain methyl-β-cyclodextrin (MBCD) in a concentration between 0.1 mM and 20 mM, or between 0.5 mM and 10.0 mM, or between 1 mM and 8 mM.

In some example implementations, a blood lysis reagent containing saponin and SPS may also contain methyl-β-cyclodextrin (MBCD) in a concentration between 1 to 5 mM per 3% w/w of saponin, or between 2 to 6 mM per 4 % w/w of saponin, or between 3 to 7 mM per 5% w/w of saponin, or between 4 to 8 mM per 6% w/w of saponin, or between 5 to 9 mM per 7% w/w of saponin.

The present inventors have observed that insufficient mixing of blood and blood lysis reagent immediately after drawing the blood is a likely cause of the formation of the filamentary structure, which is manifested as the pinkish appearance of the final elute (cell suspension at the end of sample treatment procedure). In one implementation, this issue is prevented by the addition of an anticoagulant such as ethylenediamine tetraacetic acid (EDTA) or its dipotassium salt (K2EDTA) to the blood lysis reagent. This additive will combine with calcium ions in the blood which are cofactors to many enzymatic reactions of the coagulation cascade. Although the SPS that is present in the lysing reagent is also a known anticoagulant, its activity is more driven by its direct interaction with polycationic enzymes involved in the coagulation rather than binding to calcium. Indeed, K2EDTA is specifically used in hematology tubes for collecting whole blood by inhibiting the clotting process. These tubes are designed to give final EDTA concentrations ranging from 1.4 to 2.2 mg/mL when combined with blood. Since EDTA is known to have antimicrobial properties, its concentration should be kept sufficiently low to ensure the viability of microbial cells prior to sample treatment.

In order to determine an appropriate level of K2EDTA added to the blood lysis reagent, the following experiments were performed. Four different blood lysis reagents, all containing 0.20% Tergitol™ L-62, 6.25 mM MBCD, 4.25% SPS, and 5.00% saponin in 150 mM sodium acetate buffer, but different amount of respectively, 0 mg, 4 mg, 8 mg or 16 mg of K2EDTA per 2 mL of reagent, were prepared. 2 mL of each reagent was then filled into individual tubes which were used as blood collection devices. Then into each tube, about 6 to 7 mL of whole blood was drawn from healthy human donors. Immediately after blood collection, each tube was inverted 2 times (instead of the preferred 10 times) to mix blood with the lytic reagent. Moreover, in order to promote clotting, each tube was stored at 4° C. for 4 hours before 6.5 mL of each mixture was removed and combined with 2 mL of 0.15M tris base activator in a centrifuge tube.

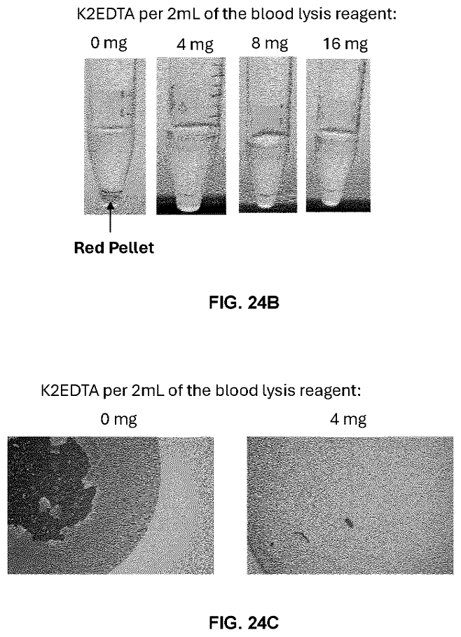

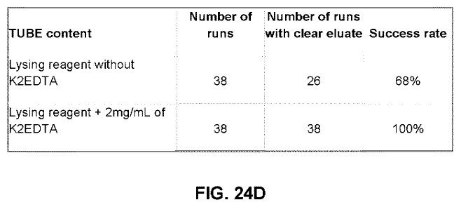

The mixtures were then subjected to centrifugal washing according to the method of Example 18. The photos of the tubes, prior to removing the 1.9 mL of supernatant is presented in FIG. 24B. As it is observed in the case of using blood lysis reagent lacking EDTA, there is a reddish pellet at the bottom part of the tube. In contrast, there is no visual sign of pellet (manifestation of the above-mentioned filamentary structure) is evident for the cases of the blood lysis reagents containing K2EDTA. Once the supernatant was removed the remaining 100 μL elute was microscopically inspected: 1 μL of the liquid was dispensed on an agar plate and was imaged with 2× objective after spontaneously spreading and air drying.

A typical photo is presented in FIG. 24C. As it is observed, there were undigested clots in the case of the blood lysis without K2EDTA, whereas the addition of K2EDTA in all amounts showed a more homogeneous distribution of particulate debris. The test was repeated with 38 blood donors and it was observed that the undesired micro-clotting effect is donor dependent. Performing visual inspection of the final elute for the signs of micro-clotting (FIG. 24D), micro-clotting was found in 32% of cases for the blood lysis reagent lacking K2EDTA, while supplying merely 2 mg/mL of K2EDTA prevented micro-clotting. Thus, employing K2EDTA in the collection tube reduces the risk of imperfect centrifugal washing during cell isolation from large volumes of blood.

In addition to acting as an anticoagulant in the lysing reagent, K2EDTA can also sequester calcium naturally present in the saponin which could lead to reagent instability such as increased turbidity before and after combining with the alkaline activator. It is also possible that, in some circumstances, the calcium from the saponin can also participate in blood clotting and lead to a red cell suspension. Thus, a controlled amount of K2EDTA in the lysing reagent can serve two functions: to provide reagent stability, and to act as an additional anticoagulant along with the SPS.

It will be understood that a wide range chelating agents may be added to the sample collection tube to facilitate the chelation (sequestering) of calcium and other ions that may be present in the sample. A non-limiting list of example chelating agents includes CDTA (1,2-cyclohexylenedinitrilotetraacetic acid), DTPA (diethylenetriaminepentaacetic acid), EGTA (Ethylenebis(oxyethylenenitrilo)tetraacetic acid), HEDTA (Hydroxyethylethylenediaminetriacetic acid), NTA (Nitrilotriacetic acid), GLDA (Tetrasodium glutamate diacetate), EDDS (Ethylenediamine-N,N′-disuccinic acid), MGDA (N-(1-Carboxyethyl)-iminodiacetic acid), IDA (Iminodiacetic acid), BAPTA (1,2-bis(o-aminophenoxy)ethane-N,N,N′,N′-tetraacetic acid), EDDHA (ethylenediamine-N,N′-bis(2-hydroxyphenylacetic acid), Disodium citrate, and Potassium oxalate. Most of these example materials belong to a class of chelation agents called aminopolycarboxylic acids. These agents use similar coordination chemistry to bind to ions as EDTA, although many are not considered to be anticoagulating agents. They can be used in various initial salt forms which provide the best solubility.

Furthermore, a non-limiting list of example substitutes for K2EDTA that act as anticoagulants includes K3EDTA, Disodium citrate, Potassium oxalate, SPS, heparin (sodium heparin, lithium heparin, or ammonium heparin).

Referring again to the flow charts illustrated in FIGS. 18 and 19, it will be understood that the direct separation of microbial cells from a whole blood sample will enable a wide variety of downstream applications, including, for example, nucleic acid amplification, sequencing, MALDI identification, antimicrobial susceptibility testing, and other applications. In some example embodiments, microbial cells separated from whole blood (or other sample types, such as those described above) according to the methods illustrated in FIGS. 18 and 19, or variations thereof, may be employed to grow and detect microcolonies, and optionally to process microbial cells harvested from microcolonies, according to any of the example embodiments described above.

In some example embodiments, at least some of the separated microbial cells may be employed for detection via a molecular amplification assay in a direct format that is without performing or requiring an intervening growth step. For example, separated microbial cells can be lysed and detected, optionally without performing nucleic acid extraction, via a suitable nucleic acid amplification method, such as any of those described above. The amplified product may be detected directly, and/or may be employed to generate a library for next generation sequencing, examples of which are described above. Indeed, any one or more of the nucleic acid amplification and/or sequencing methods and workflows described above in relation to the use of microbial cells derived from harvested microcolonies may be employed to process microbial cells separated from whole blood samples (or other sample types, such as those described above) according to the methods illustrated in FIGS. 18 and 19, or variations thereof.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Summary of Experimental Studies and Results

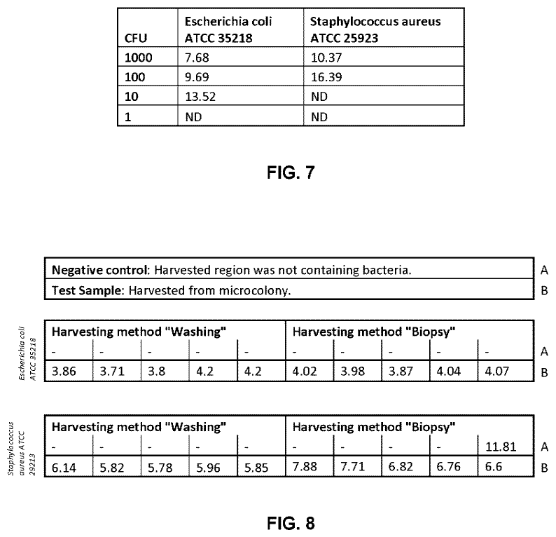

Bacterial suspensions were prepared with approximately known concentrations in 1 mM PB buffer and tested them by rRT-LAMP according to the methods of Example 9. The results, which are presented in FIG. 7, indicate that the assay sensitivity for clean samples is 10 and 100 CFU, respectively for Escherichia coli ATCC 35218 and Staphylococcus aureus ATCC 25923. For cell suspensions obtained for whole blood samples, the detection limits will be even higher due to the presence of assay inhibition by the sedimenting blood debris.

After determining the sensitivity of the rRT-LAMP assay, the 4 hours-old microcolonies were harvested by biopsy and washing methods of Example 8 and performed rRT-LAMP according to the method of Example 9. The result, which is presented in FIG. 8 indicates the equivalency of the two harvesting methods. In addition, the reported times of positivity are under 10 minutes. This type of TTP is well below the TTP which would result from environmental contamination.

Assays were also performed employing the swapping method of microcolony harvesting according to Example 8D. The resulting TTP (averaged over 3 replica) are presented in FIG. 9A for 10 different bacterial species (four Gram negative: Escherichia coli, Pseudomonas aeruginosa, Acinetobacter baumannii, Klebsiella pneumoniae; six Gram positive: Staphylococcus aureus, Streptococcus pyogenes, Enterococcus faecalis, Staphylococcus epidermidis, Streptococcus pyogenes, Staphylococcus heamolyticus). Again, the measured TTPs are in sub-10 minutes range, indicating the robustness of the test against environmental contamination.

The suitability of harvesting microbial cells from microcolonies for downstream molecular assay was also illustrated by running PCR assay. In this case, rRNA gene on the genomic DNA was selected as target and specific PCR primers were designed for Staphylococcus aureus ATCC 25913. The 4 hour and 6 hour old microcolonies were harvested by swabbing method of Example 8C and cell suspension in 1 mM PB buffer was made. As negative control an area of plate that had no microcolonies was swabbed in a manner mimicking the method described in Example 8C. The cell suspension was lysed and tested with the PCR assay according to the method of Example 10. The result is shown in FIG. 10A.

In another investigation, a microcolony was harvested, grown for 4 hours, employing the swapping method of Example 8C, into 200 μL of PB buffer. The cell suspension was split into two 100 μL aliquots. PCR assays were performed on the two aliquots according to the method of Example 10, one aliquot subjected to heat lysis and the other not lysed before adding the sample with the master-mix. The result, which is presented in FIG. 10B, indicates that the two assays had similar performance as quantified by CT values. Again, this unexpected observation is beneficial considering that not involving any type of cell lysis simplifies the assay method and reduces the chance of performance deterring events, such target loss and/or introducing inhibitors and contaminants, during the cell lysis step. In addition, the glass bead beating, which is often employed for lysis of Gram-positive cells, shears gDNA and breaks it into small pieces. In contrast, not involving cell lysis, or employing mild lysis approaches, such as heat lysis or electrical lysis, keeps the integrity of gDNA. This is particularly beneficial for sequencing applications.

### Example 1A: Rapid Gram Staining of Microbial Cells Harvested from Microcolonies

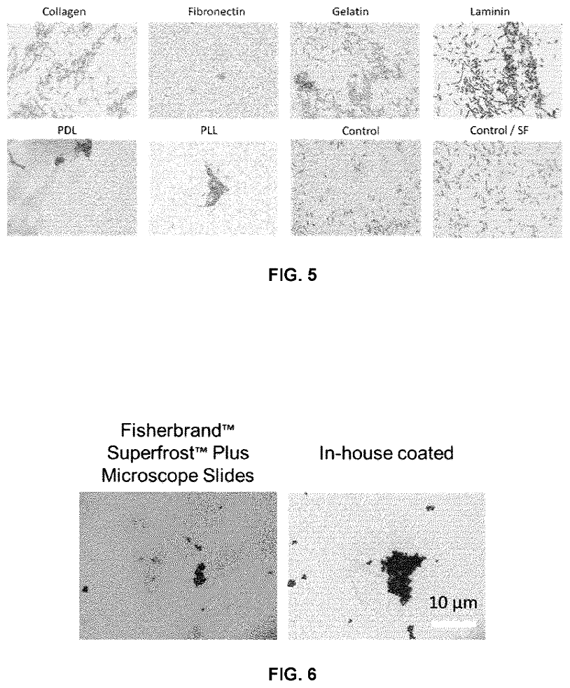

Affinity surfaces for harvesting microbial cells from microcolonies were prepared by cutting from the mentioned slides and verified their efficacy by performing Gram staining according to the methods of Example 7B for all bacterial targets listed in FIG. 4. One sample result, for the case of E. coli microcolony grown for 4 hours after cell suspension spreading, is presented in FIG. 5. As qualitatively judged, in terms of transfer efficacy Fisherbrand™ Superfrost™ Plus Microscope Slides (Control SF), Rat tail Collagen I (Collagen), Mouse Laminin (Laminin) and Gelatin were comparably good. Among these Laminin has been more prone to staining artifacts such as false colouring. The cells transferred by the PLL-coated surface were not dispersed for Gram staining.

Another embodiment of an affinity surface was prepared following the method of Example 7A. The result for the case of Staphylococcus aureus microcolony is presented in FIG. 6.

### Example 2: Microbial Cell Culture Preparation

Gram-positive bacteria except for Staphylococcus aureus and Streptococcus pneumoniae cell culture was prepared as follows:
1. Thirty μL of respective bacteria species and strain glycerol stock was inoculated in 3 mL of tryptic soy broth (TSB) and incubated at 37° C. for overnight with shaking at 150 rpm.
2. Tenfold diluted culture in TSB was incubated at 37° C. for 1 hour (Enterococcus faecalis, Enterococcus faecium and Streptococcus agalactiae) or for 2 hr (Staphylococcus epidermidis).

Gram-negative bacteria except Pseudomonas aeruginosa cell culture was prepared as follows:
1. Thirty μL of respective bacteria species and strain glycerol stock was inoculated in 3 mL of TSB and incubated at 37° C. for overnight with shaking at 150 rpm.
2. Tenfold diluted culture in TSB was incubated at 37° C. for 1 hour (Acinetobacter baumannii, Enterobacter cloacae complex, Escherichia coli, Klebsiella pneumoniae, and Proteus mirabilis) or 2 hr (Serratia marcescens).

Staphylococcus aureus cell culture was prepared as follows:
Thirty μL of respective strain glycerol stock was inoculated in 3 mL of TSB and incubated at 37° C. for 3 hr with shaking at 150 rpm.

Streptococcus pneumoniae cell culture was prepared as follows:
Thirty μL of respective species or strain glycerol stock was inoculated in 3 mL of TSB and incubated at 37° C. for 3 hr with shaking at 80 rpm in the presence of CO₂ generating pouch.

Pseudomonas aeruginosa:
1. Six μL of Pseudomonas aeruginosa strain glycerol stock was streaked on tryptic soy agar (TSA) with 5% sheep blood plate and incubated at 37° C. for overnight (P1).
2. The bacteria colony was subcultured one more time on agar plate (P2).
3. One colony from the plate was inoculated in 3 ml of TSB and incubated at 37° C. for 3 hours with shaking at 150 rpm.

Based on OD measurements, serial dilutions of the respective bacteria were prepared in TSB at a nominal concentration of 1000 CFU/mL.

### Example 3: Preparation of Agar-Based Solid-Phase Growth Media Plates

Agar-based solid-phase growth media plates were prepared with final agar concentrations within the range of 1.75% w/v.

Composition of the base/dehydrated culture media (BBL2 from BD): Pancreatic digest of Casein 14.5 g/L, Papaic digest of Soybean Meal 5 g/L, Sodium Chloride 5 g/L, Agar 14 g/L, Growth Factors 1.5 g/; 4 g of BBL2; 0.35 g of agar; 95 mL of distilled water; 5 mL of sterile defibrinated sheep blood.

The preparation steps were as follows:
1. Heat the TSAB powder in molecular biology grade water on a hot plate at 100° C. inside a water bath for 10 mins.
2. Add TSAB to water,
3. Heat to boiling point and stir well.
4. Autoclave the solution (120° C. for 15 minutes)
5. Cool down to 55° C., add blood, and stir well.
6. Pre-warm the pipette with warm water.
7. Dispense onto plates and let solidify for 5 mins.

### Example 4A: Microcolony Growth on Solid Growth Media

The cell suspension of Example 3 was dispensed on the plate of Example 4 (d=55 mm plate) and was spread with an L-shaped spreader (Global Scientific) over an area of ~10 cm². The plate was incubated into a homemade incubator (T=36° C., saturation humidity) and was scanned by a metallurgic microscope with a 2× objective once every 30 minutes.

### Example 4B: Spreading

### Spiral Plating

Spreading via spiral plating employes using an instrument used to dispense a liquid sample onto a Petri dish in a spiral pattern. The spiral plater rotates the dish (or move the dispensing tip) while simultaneously dispensing the liquid and either linearly moving the dish or the dispensing tip. The spreading can be done as following:
1. Fill the dispensing tip with the concentrated bacteria suspension which you are going to spread.
2. Place the dispensing tip at the center of agar plate.
3. Rotate the agar plate while simultaneously dispensing the liquid and linearly moving the dispensing tip from the center to the periphery of agar plate.

### L-Shape Spreader

The spreading can be done as following:
1. Using a micropipette, place concentrated bacteria suspension onto the center of the agar plate without touching the agar.
2. Using your sterile spreader (L-shape), gently rub across the agar surface while rotating the plate with the other hand.
3. Continue spreading until the liquid is absorbed by the agar.
4. After completing the spread, decontaminate the spreader with alcohol or bleach, and dispose of the spreader in the waste beaker.

### Example 4C: Image Registration in Spatial-Frequency Domain

1. Convert images to binary using Otsu's method.
2. The Discrete Fourier Transform (DFT) of the two images is computed.
3. The complex conjugate of the DFT of the second image is computed.
4. The DFT of the first image and the complex conjugate of the DFT of the second image are multiplied together. This operation is also known as cross-power spectrum.
5. The Inverse Discrete Fourier Transform (IDFT) is then computed from the cross-power spectrum, which gives the cross-correlation in the spatial domain.

6. The location of the peak in the cross-correlation corresponds to the displacement between the two images.

### Example 5: Microcolony Detection and Localization

The image registration step was used for microcolony detection. By aligning the successive images, the object detection algorithm compared and matched the features of the objects (blood debris), leading to more accurate and reliable detection results. To align imaging data acquired at different time points 2D-2D registration (with affine transformation constraints) was performed on each image. Image registration was based on the detection and extraction of the local characteristic features such as corners, borders, and/or blobs. The SURF (Speeded-Up Robust Features) detector was used and features were extracted from the 2-D gray-scaled input images. During image registration, two images of the same scene taken from at different times were aligned. The goal was to find a transformation that maps the pixels in one image to their corresponding locations in the other image based on the prior knowledge of that transformation. By incorporating prior knowledge into image registration, the resulting transformation was more accurate and robust. Intensity features present at one or both were classified as background while intensity features appearing on further images were classified as foreground. Objects >250 pixels present in the foreground were classified as microcolonies. The recorded coordinates of the detected microcolonies were used as the location.

### Example 6: Preparation of Affinity Surface for Microcolony Harvesting

In this example, a method is presented for coating a glass surface, which can be used as the affinity surface for microcolony harvesting.

### Material Used:

1 L deionized H2O, 5 g gelatin (G6144-100G Gelatin from porcine skin, Sigma-Aldrich), 0.5 g chromium potassium sulfate dodecahydrate CrK(SO4)2·12H2O (243361-5G Chromium(III) potassium sulfate dodecahydrate, Sigma-Aldrich).

The preparation steps:
1. Heat 1 L of deionized H₂O to 45° C.
2. Add 5 g of gelatin and mix with a magnetic stirrer until the gelatin crystals disappear.
3. Add 0.5 g of chromium potassium sulfate dodecahydrate and keep stirring until the solution should turn a pale blue.
4. (Optional step): Add a few crystals of thymol as a preservative.
5. Cool down the solution to room temperature.
6. Filtered the solution to remove any big particles (if present).
7. Place glass slides (affinity surfaces that need to be coated) in a suitable slide holder (e.g. Thomas Scientific Part #8542K20).
8. (Optional step): Rinse slides with hot water to wet.
9. (Optional step): Place slides in hot soapy water for 10 min and occasionally agitate them.
10. (Optional step): Rinse slides in hot water until all traces of soap are gone. (~15 min.)
11. (Optional step): Rinse slides under running distilled water (~5 min).
12. (Optional step): Dip slides in 70% EtOH. Drain excess ethanol and dry in a dust-free area overnight. Set slides in the rack at an angle with absorbent paper underneath and cover using a large container, or tin foil.
13. Dip racks of clean slides in the warm gelatin solution (40-50° C.): 3 to 5 times (~5 seconds each).
14. Remove the racks containing the slides and let the excess gelatin to drip
15. Air dry the slides in a dust-free environment for 24 hours.

### Example 7: Gram Staining of Cells Harvested From Microcolony

### Materials:

Gram Stain Kit: Gram Crystal Violet, Gram lodine or Stabilized Gram, lodine, Gram Decolorizer, and Gram Safranin or. (Cat. No 212539, BBL, BD) Procedure:
1. Gently press the affinity surface over the area with the detected microcolony.
2. Move the surface away and allow it to air dry.
3. Methanol fixes the affinity surface by flooding it with absolute methanol (for 1-2 min).
4. Allow the affinity surface with microcolony to air dry.
5. Flood the affinity surface with primary stain (Gram Crystal Violet) and wait for 1 min.
6. Remove the primary stain by dipping it into cold water and gently agitating
7. Dip the slide into mordant (Stabilized Gram lodine) and wait for 1 min.
8. Remove the mordant by dipping the affinity surface into cold water and gently agitating.
9. Dip the affinity surface into Gram Decolorizer and wait for 30 sec.
10. Wash the affinity surface by dipping it into cold water.
11. Dip the affinity surface into counterstain (Gram Safranin) and wait for 60 sec.
12. Wash the affinity surface by dipping it into cold water.
13. Air dry.

Examine the affinity surface under an oil immersion lens (100×).

### Example 8: Examples of Microcolony Harvesting Methods

The following examples illustrate non-limiting specific implementations of some of the harvesting methods described above.

### Example 8A: Example of Biopsy Method of Microcolony Harvesting

This method involves using a very small biopsy punch (or pipette tip as its substitute) to extract/biopsy a small part of solid growth media that includes the detected/located microcolony. An example implementation of this method is described as follows:
1. Use a pipette with a 20-200 μL tip pre-filled with 100 μL of phosphate buffer (PB).
2. Bring down the tip as close as possible above the microcolony.
3. Push down the tip in a manner that the gel is cut (not squeezed). // The cut part of the gel goes inside the tip.
4. Move up the tip and dispense the cut microcolony along with PB to a tube.
5. After vortexing the tube for 10 sec, solid growth media material (cylinder) is separated from PB (pipette content) with methods such as brief centrifugation or coarse filter.

The remaining PB is the final cell suspension.

### Example 8B: Example of Washing Method of Microcolony Harvesting

This involves washing bacterial cells off the gel surface with a buffer that is consistent with the downstream assay. An example method is as the following:
1. Aspirate 10 μL of the buffer.
2. Bring the tip above the detected/located microcolony and hold it close to the surface.
3. Dispense the buffer.
4. Immediately gently touch the surface with the tip.
5. Scrape the surface to dislodge the bacterial cells.
6. Aspirate the buffer.
7. Transfer the content to a sterile tube.

### Example 8C: Example of Swabbing Method of Microcolony Harvesting

An example method for swabbing used VITEK® PICKME™ Pen (bioMerieux).
1. Pick a nib with a PickMe pen.
2. Bring it above the detected/located microcolony.
3. Gently touch the area with the nib.
4. Move the nib over the surface a few times.
5. Move the nib inside a tube containing a buffer appropriate for the downstream assay.
6. Agitate the nib to release the cells.
7. Eject and trash the nib.

### Example 8D: Example of Suction Method Employing Conductive Pipette Tips

One implementation of microcolony harvesting by suction employs conductive pipette tips, which provide accurate positioning of tip with respect to the gel surface. The steps are as the following:
1. The microcolony is detected and located.
2. The conductive pipette tip is picked up.
3. 5 μL of 1 mM PB buffer is aspirated.
4. The tip is brought above the microcolony.
5. Feedback from electrical positional sensing ensures the actual position of the tip with respect to the gel surface.
6. The tip is lowered down by ~0.3 mm and pressed to ensure a good seal with the gel surface.
7. The harvesting buffer is dispensed into the gel.
8. Aspirate the dispensed harvesting buffer to draw the microcolony into the tip.
9. The microcolony location may be microscopically inspected to verify successful harvest.

### Example 8E: Example of Electrical Method for Microcolony Harvesting

An example method for harvesting, employing an electrical pin, is as the following
1. Select a pin and apply an electrical isolating layer of 1 Mil Kapton® HN film.
2. Connect the pin to the positively charged electrode.
3. Bring the pin above the microcolony.
4. Apply V_{g}=18 V (with respect to the gel).
5. Bring down the pin to touch the gel surface containing microcolony and keep the microcolony in contact with the pin for minimum 10 sec.
6. Bring the pin above the substrate or buffer onto/into which the microcolony is to be dispensed.
7. Apply Vₛ=−12 V (with respect to the substrate or buffer).
8. Bring down the pin to touch the substrate or buffer.

Example 8F: Assessing the Reproducibility of Microcolony Harvesting

In order investigate the reliability of the suction method of harvesting, the following experiment was performed:
1. Receive target microbial cells (10⁶ CFU/mL in TSB) from cell culture and prepare serial dilutions for spiking to 10⁴ CFU/mL in TSB.
2. Spike 5 μL (nominal 50 CFU) to 100 μL of media.
3. Spread the cell suspension onto an agar plate.
4. Incubate in 35° C. incubator.
5. Image once every hour to detect microcolonies and determine their location.
6. After 5 hours of incubation:
(a) Randomly select one of the microcolonies and, harvest it into 5 μL of saline.
(b) Dispense and resuspend the harvest into 100 μL of saline ( 1/20 dilutions of one colony pick) in a microtube for plate counting of the biomass.
(c) Image the microcolony's location to check if the action was successful.
(d) Repeat the steps above for two more microcolonies.
7. Dilute the cell suspension in step 6(b) to 1/100 or 1/1000 dilutions of one colony pick.
8. Plate 100 μL each of step 8 in triplicates.
9. Incubate the plates overnight.
10. Count the colonies and calculate the total biomass per one colony pick.
11. Assess the microcolony biomass and its variation across the three replicas.

### Example 9: Real-Time RT-LAMP Assay

The cells were lysed by heating the cell suspension to 95° for 10 minutes in a GeneAmp® PCR System 9700 (Heat lysis method). 5 μL of the lysate was added to reaction wells of the cartridge of a LightCycler® 96 Instrument at bottom of which RT-LAMP enzymes (New England Biolabs, WarmStart® RT-LAMP Lyo-Ready Kit) which were dried. After rehydration, the liquid was transferred to another well on the plate at bottom of which a mixture containing STYO™ 9 dye and a set of RT-LAMP target-specific primers had been dried. The plate was sealed and placed inside the LightCycler® 96 Instrument and was heated to 64° C. and kept the temperature fixed 20 minutes. The time at which the dynamic fluorescence signal exceeded the background was taken as Time-to-Positivity (TTP).

### Example 10: Real-Time PCR Assay

The cells were lysed either by heating the cell suspension to 95° for 10 minutes in a GeneAmp® PCR System 9700 (Heat lysis method), or by electrical lysis method of Example 11. A 1 μL portion of the lysate was combined with 4 μL of a master mix containing STYO™ 9 dye and a set of target-specific PCR primers (targeting rRNA genes), and polymerase enzymes (Kappa HotStart, Roche). The mixture was pipetted into the PCR plate which was then placed in a LightCycler® 96 Instrument. The 38 PCR cycle consisted of 30 s extension at 67C and 5 s melting at 95° C. The CT value was determined by the instrument based on the real time fluorescence signal.

### Example 11: Electrical Lysis of Cell Suspension

The electrical lysis method used in this example employed the e-Lysis™ method developed by Qvella®. The technique has been described in International Patent Application No. PCT/CA2012/000698. Briefly, the lysis chamber had a volume of 20 μL and a thickness of 200 μm, with top and bottom electrodes. The electrodes were formed from microstructured aluminum with a conformal aluminum oxide dielectric layer, as described above. When the chamber was filled with the cell suspension, a series of square wave AC pulses of duration 50 μs and amplitude of 200 V, were applied a duration 50 ms.

### Example 12: Dependence of Surface Artefact Density on Blood Lysis Regent Composition

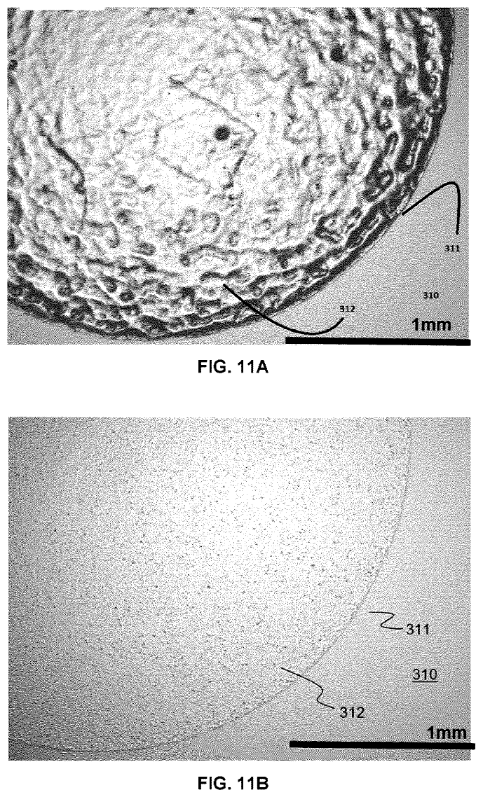

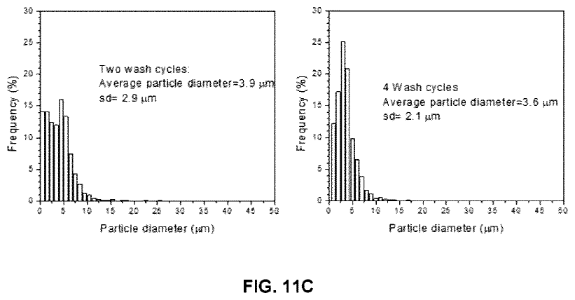

In order to illustrate the dependence of the surface artefact density on the composition of the blood lysis reagent, 4 mL of whole blood sample was processed according to the method of Example 13, both with 2 washing cycles using a blood lysis reagent having a composition as the following: 35 mg/mL saponin, 20 mg/mL SPS (BLR1) and (ii) 35 mg/mL saponin, 20 mg/mL SPS, 0.3% w/v Triton™ X-100, and 50 mM carbonate-bicarbonate buffer, with a pH of 10 (BLR2). After exposure of the sample to the respective blood lysis reagents and centrifugal separation, 1 μL of each final microbial cell suspension were pipetted on a spot-on agar gel plate. The microbial cell suspension samples spread to circular areas with diameters of about 5 mm and air dried in about three minutes. These areas, which herein are labeled as mini-culture region (MCR) were imaged by a microscope equipped to a 5× objective and was presented in FIGS. 11A and 11B. In these figures the MCR, unused agar plate surface and the boundary between two regions are respectively indicated by 310, 312, and 311. As it is observed the inclusion of Triton X-100 and carbonate-bicarbonate buffer significantly reduces the background (density of surface artefacts).

The present inventors found that this background level could not be significantly reduced further by increasing washing cycles. For example, this was demonstrated by treating a 4 mL whole blood sample using BLR2 having a formulation as described above with 2 or 4 subsequent centrifugal wash cycles. A 1 μL of the resulting microbial cell suspension was dispensed on the agar plate and allowed to spread and air dry. The pictures of the resulting MCRs were recorded with a 10× microscopic objective, and were analyzed for the size distribution of debris at the end of 2 and 4 wash cycles. The particles were located via intensity-based adaptive auto-threshold methods and were fitted with ellipses. More precisely, image segmentation was performed and a label was assigned to every connected group of pixels in an image such that pixels with the same label share certain intensity characteristics. The histogram of the measured major particle diameter (major axis of a fitted ellipse) distribution is presented in FIG. 11C. As it is observed, the distribution plots are qualitatively similar, despite the fact that the sample is diluted by a factor of 400-fold between 2 and 4 washes.

### Example 13: Sample Treatment of 4 mL Spiked Whole Blood Samples

Sample preparation was performed for spiked whole blood samples as follows:
1. In a 15 mL centrifuge tube, 4 ml of blood lysis reagent was added to 4 ml of spiked whole blood sample.
2. The centrifuge tube was mixed by vortexing for 1 minute at maximum speed of the vortexer.
3. The centrifuge tube was centrifuged at 4000 rpm for 8 minutes.
4. A supernatant of 7.9 ml was removed.
5. The first wash cycle was performed, by adding 2.9 mL of wash buffer to the residue, mixing the solution was mixed by gently vortexing, centrifugation at 4000 rpm for 3 min, and withdrawing and discarded 2.9 mL of supernatant such that 100 μl of residual liquid was retained.
6. The second wash cycle was performed, by adding 2.9 mL of wash buffer to the residue, mixing the solution was mixed by gently vortexing, centrifugation at 4000 rpm for 3 min, and withdrawing and discarded 2.9 mL of supernatant such that 100 μl of residual liquid was retained.
7. The third wash cycle was performed, by adding 1.9 mL of wash buffer to the residue, mixing the solution was mixed by gently vortexing, centrifugation at 4000 rpm for 3 min, and withdrawing and discarded 1.9 mL of supernatant such that 100 μl of residual liquid was retained.
8. The fourth wash cycle was performed, by adding 1.9 mL of wash buffer to the residue, mixing the solution was mixed by gently vortexing, centrifugation at 4000 rpm for 3 min, and withdrawing and discarded 1.9 mL of supernatant such that 100 μl of residual liquid (cell suspension) was retained.

### Example 13: Growth and Detection of Microcolonies

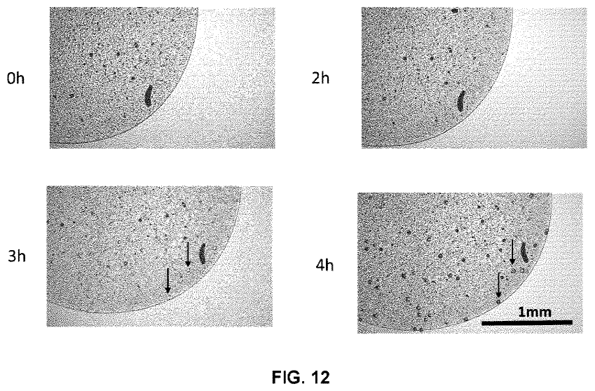

In order to illustrate an example of the dynamic nature of microcolony formation based on microbial cells obtained directly from a whole blood sample, a 4 mL whole blood sample was spiked with 3000 CFU of Proteus mirabilis (PM) cells and treated according to the method described in Example 12 below. One μL of the resulting cell suspension was dispensed on each of four agar plates and was allowed to spontaneously spread to a circular area with a diameter of ~5 mm, which is henceforth referred to as a “mini culture” region (MCR). Images of a portion of the resulting MCRs are presented in FIG. 12. By visual inspection of the images at 3 and 4 hours following the onset of incubation, some microcolonies, which are indicated by arrows, can be observed. However, in order to detect microcolonies at shorter incubation times, for example within 2 hours, the images may be analyzed for differentiating the microcolonies, marked by arrows, from the background.

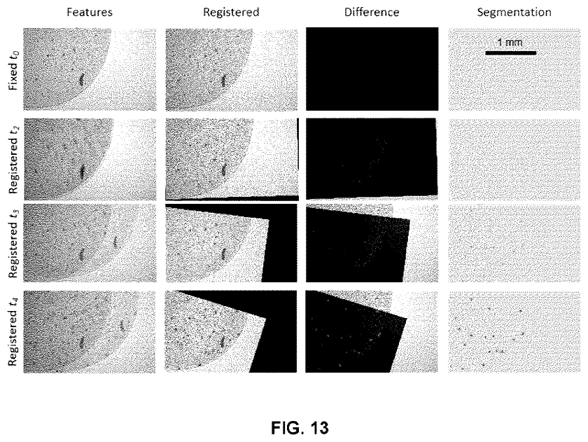

One example method for microcolony monitoring is described as follows. As is observed from FIG. 13, in order to align imaging data acquired at different time points (0, 2, 3 and 4 hours after seeding, as shown in the figure), 2D-2D registration (employing translation and rotation) with rigid transformation constrains was performed. The corresponding intensity feature points between the t₀=0 hours image and each further image (t₂=2 hours, t₃=3 hours, t₄=4 hours) were automatically identified using the key-point detector SURF and used for aligning imaging date with respect to to. Intensity features present at to were classified as background while intensity features appearing on further images (cells/bugs) were classified as foreground. The position of given individual microcolonies have been marked in consecutive images.

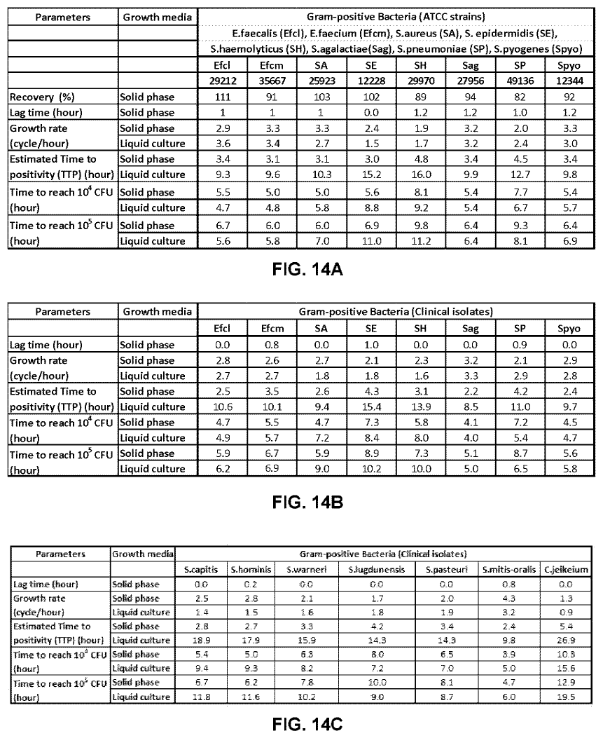

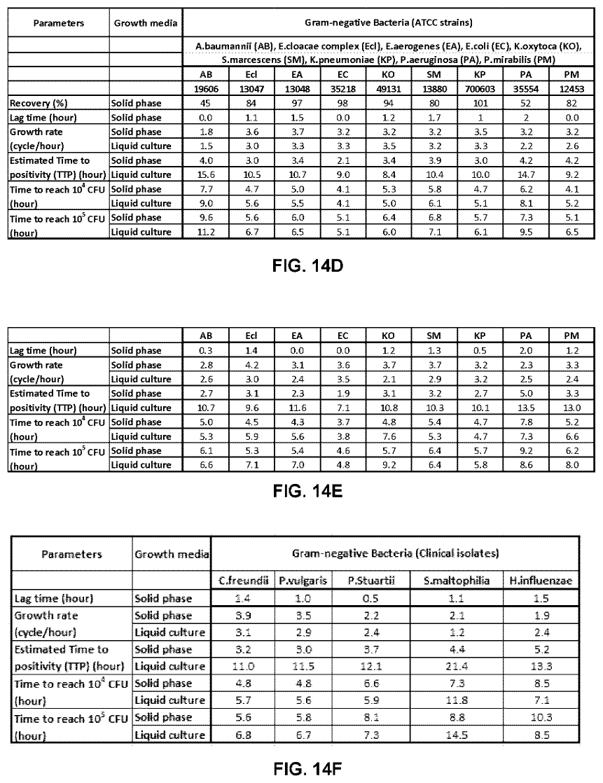

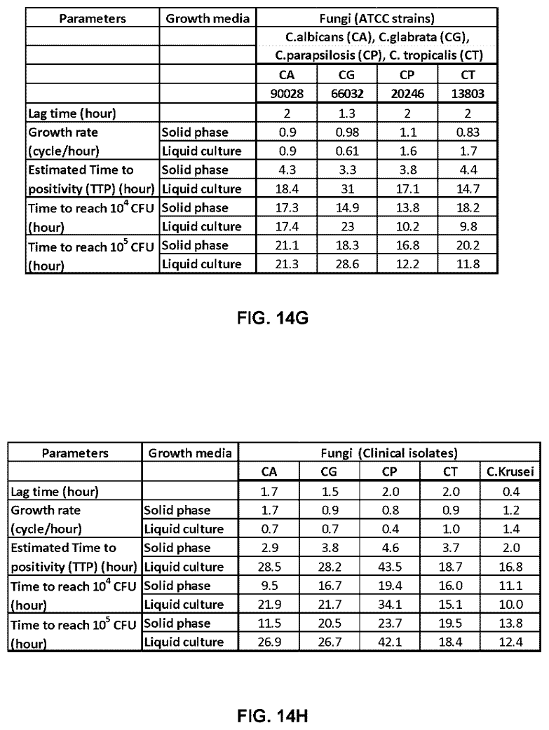

The determination of the background allows for enhanced detection of microcolonies. For instance, despite the unusually large translational and rotational offsets between the 4 acquired images in the case of FIG. 7, the identification of the colonies at t₃=3 hours after incubation is unambiguous. Using this method, it becomes easier to develop fully automated microcolony identification (reduction of time to positivity TTP) and tracking system for screening the image sequences of the unstained living microorganisms. The robustness of method is illustrated below, in connection with results presented in FIGS. 14A to 14H, in comparison with estimates based on the aforementioned background thresholding method (e.g. declaring a spot a microcolony if its radius R>R_{threshold}=R_{back.av}+n*sd).

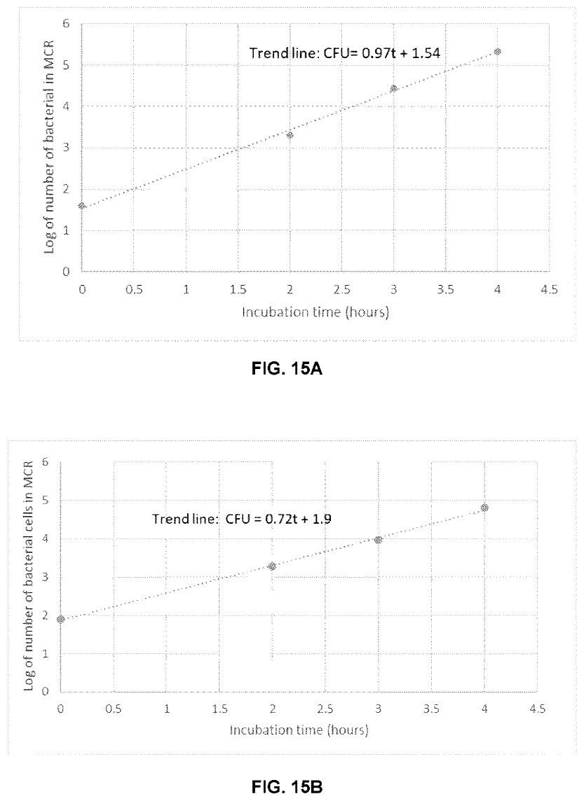

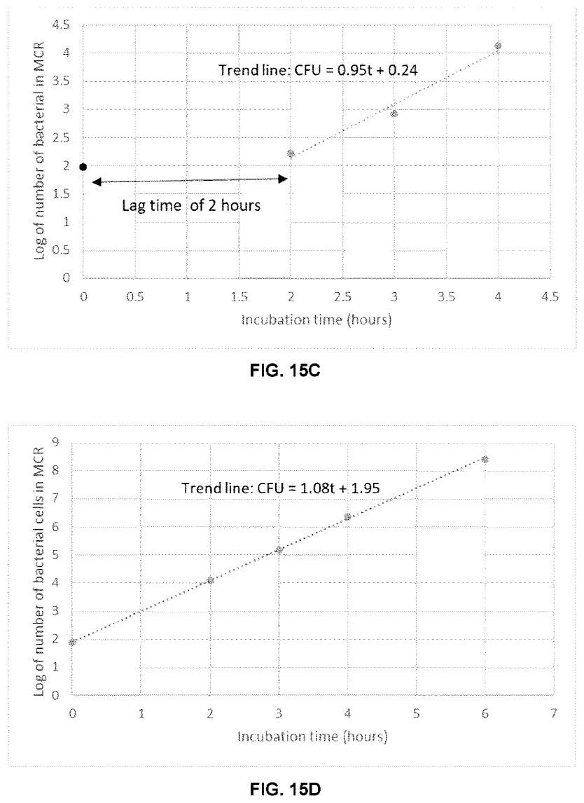

The growth rate on the solid growth medium (gel) was determined following the steps of Example 14 below. The number of colonies forming units in the MCRs were counted and its logarithm (Log(CFU)) was plotted versus incubation time, as presented in FIG. 15A for the case of Proteus mirabilis (PM). The slope of this curve, i.e. 0.97, is used to calculate the growth rate, through the relation growth rate=slope/log(2)=3.23 cycles/hour. As another example, the growth rate of Staphylococcus epidermidis (SE) was measured and the measured v(Log(CFU)) versus incubation is shown in the time plot in FIG. 15B. The calculated growth rate is 2.4 cycles/hour. This growth rate is about 1.5 times higher than the measured growth rate of Staphylococcus epidermidis incubated on a CMOS chip [Jung, Jae Hee, and Jung Eun Lee. “Real-time bacterial microcolony counting using on-chip microscopy.” Scientific reports 6 (2016): 21473.]. Depending on the preparation method of the stock solution, the seeded microbial cells may pass through a lag phase before proliferation to the microcolony. For instance, as it is observed from FIG. 15C, Pseudomonas aeruginosa (PA) cells show about 2 hours of lag time. The linear trend in semi-logarithmic scale, is expected to continue while the number of cells in a colony is sufficiently low such that most of cells can divide. Once the number of cells at the inner region of the microcolony, which are deprived of space for proliferation, exceeds the number of cells at the periphery, the overall growth rate of the microcolony is expected to decrease. Such deviations have not been observed by present inventors for microcolonies containing as many as 10⁶ cells in the case of E. coli, as illustrated in FIG. 15D. Accordingly, using growth rate and lag time data to estimate the times required for reaching desired cell number appears to be justified.

FIGS. 14A-14F present the measured time lag and growth rate of seeded cells by microcolony detection for a collection of microbial cells species that constitute the majority of pathogenic microbes that are typically encountered in blood stream infections. The table also includes the measured cell recovery fraction, i.e. the fraction of cells that are successfully separated from the spiked blood sample and resuspended in a cell suspension while maintaining their viability, and are thus able to form colony, determined according to the method of Example 15. In addition, the table also presents the estimated time to positivity (TTP) for the present example growth methods involving colony growth on a solid growth medium (“solid phase”), as determined by the time at which the microcolony is discernable relative to the background (using the example methods disclosed above).

The TTP will be affected by the sensitivity of the detection method and its associated analysis and the background. In the simplest case of interrogating the presence of a microcolony grown from the microbial cells which have been separated from a whole blood sample by employing the simple size selection method described above, the TTP can be estimated as follows. The threshold size R_{threshold}=R_{back.av}+n*std was calculated using the parameters in the case of 2 washes in FIG. 11C: R_{hreshold}=2+3*1.5=6.5 μm. Assuming the worst-case scenario of closed packing and an average bacterial size of 1 μm², the number of cells inside a circle with radius R_{threshold} will be about 120 CFU. Thus, TTP=T_{lag}+7/growth rate.

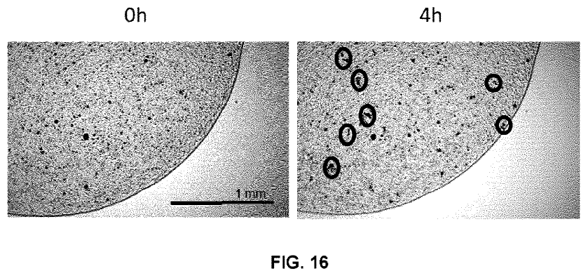

In the case of fungal species, as a consequence of their large size relative to bacteria, a single division that results in a binary division may be sufficient to detect a microcolony and arrive at a determination of positivity. This is illustrated in FIG. 16, which shows the time-lapse images of a section of a blood agar plate, on which 1 μL of a microbial cell suspension containing microbial cells separated from a whole blood sample had been dispensed. As can be calculated from growth-rate data in FIGS. 9G and 9H, after approximately 4 hours of incubation, the number of cells has increased by a factor of ~3. The proliferation of fungal cells is easily recognized comparing two photos. Thus, it is concluded that in a large distribution of fungal cells the time to positivity is TTP=T_{lag}+1/growth rate. In a typical blood sample, the number of cells in single digits and the Poisson statistics cannot be ignored. Thus, the formula should be replaced by TTP=T_{lag}+n/growth rate, where n is larger than one. In one example implementation, a value of n=2 was employed.

The characteristic growth rates are comparable with growth rate in planktonic state. In order to illustrate this concordance, the growth rate in liquid culture was estimated from experiments that were performed as described below. Ten mL samples of whole blood, spiked with different strains of microbial cells at a concentration of 5 CFU/mL, were inoculated into respective BacT/ALERT® FA Plus culture bottles and incubated in BacT/ALERT® VIRTUO. After the incubator indicated positivity, a 1 mL aliquot was drawn from each bottle, serially diluted, and plated for determining number of CFUs. Ignoring lag time, and assuming that the growth rate is constant, the growth rate was estimated based on the initial spiked concentration ratio and the final bacterial concentration at positivity by plate counting, and time to positivity (TTP). As it is observed from FIGS. 14A-14H, the growth rates on solid growth media and in liquid phase growth media are similar. However, as can be clearly appreciated by the TTP values, the solid phase is advantageous as a consequence of the localized nature of the microcolony, facilitating detection on the solid phase at a much earlier time. For example, while most bacterial species are easily detected in ~3 hours after plating according to the present microcolony example method, the TTP for incubation in a culture bottle is typically above 10 hours.

FIG. 14A-14G also includes estimates of the time needed for a bacterial cell to result microcolonies having 10⁴ and 10⁵ cells. These quantities are relevant for performing subsequent microbial identification and/or antimicrobial susceptibility testing, as discussed below.

In order to characterize the performance of the present example method for the rapid and direct formation and detection of microcolonies, two characteristics of common pathogens found in blood stream infections were measured, namely (i) lag time and (ii) growth rate. The recovery fraction of these pathogens from blood samples was also measured.

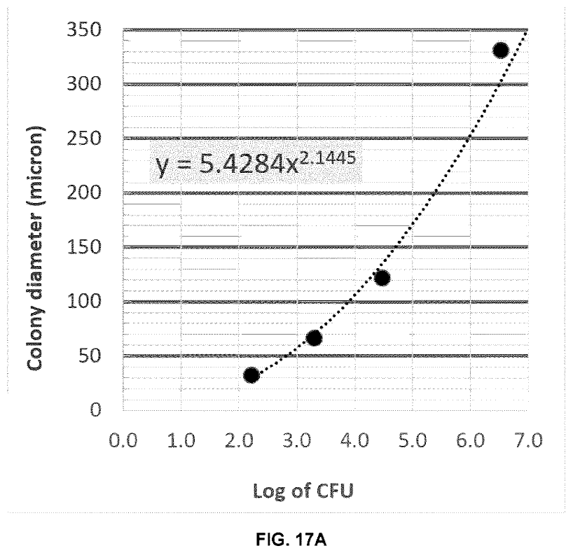

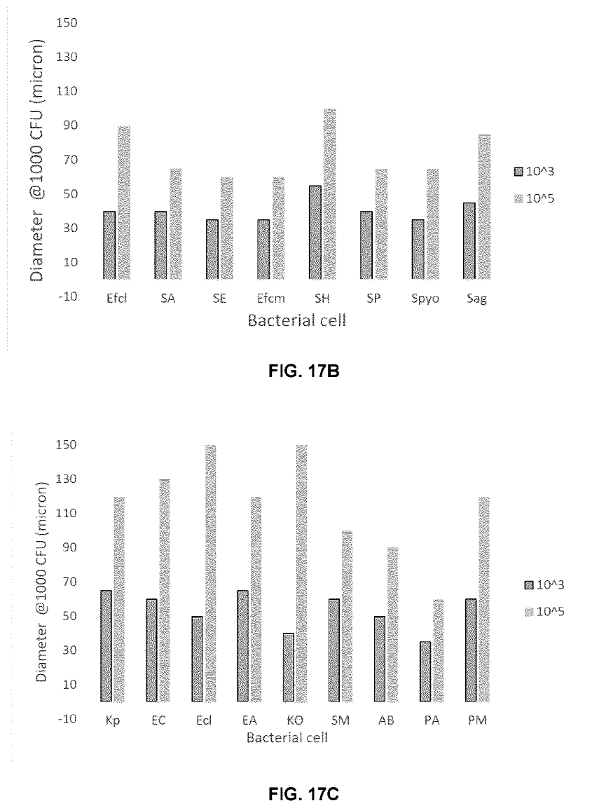

As illustrated below in FIGS. 17B and 17C, a selected size threshold (e.g. diameter threshold) may be employed to ensure that a sufficient number of microbial cells are harvested for a wide variety of cell classes (e.g. species). For example, as shown in FIGS. 17B and 17C, at least 10³ microbial cells can be obtained across a wide range of microbial cell species provided that a microcolony is harvested after reaching a diameter threshold of approximately 50 μm.

Likewise, as shown in FIGS. 17B and 17C, at least 10⁵ microbial cells can be obtained across a wide range of microbial cell species provided that a detected microcolony is harvested after reaching a diameter threshold of approximately 150 microns (e.g. prior to reaching a diameter of 180, 190, 200, 250 μm). Although the present example threshold embodiment refer to a diameter, it will be understood that other size measures, such as a radius or area, may be employed in the alternative.

FIG. 17A plots the measured dependence of microcolony diameter on cell content for the example case of E. coli (obtained according to the method described in Example 14). The scatter graph has been fitted by a power law trend line, using which the average microcolony diameters with cell contents of 10³ and 10⁵ cells have been calculated to be respectively 60 μm and 170 μm. Following a similar approach, the average diameters at 10³ and 10⁵ CFU cell content was calculated for 17 prevalent pathogenic gram positive and gram-negative bacteria and presented the results respectively in FIGS. 17B and 17C. According to this information, if a bacterial microcolony is harvested when its diameter reaches 65 μm, regardless of its identity, the number of microbial cells in the microcolony will be likely be within the range of 10³ to 10⁵ CFU.

### Example 14: Measuring Growth Rate and Colony Size of Microbial Cell on Agar Plate

The growth rate of a microbial cell on an agar plate is determined through the following steps:

Prepare starting cell suspension with a nominal concentration of 10⁵ CFU/mL.

Dispense 1 μL of the cell suspension on one of three identifiable regions on an agar gel plate and allow them to spread over a mini-culture region (MCR) and air dry. Thus, there will be 3 MCR, identified as MCR1, MCR2, and MCR3, on the plate.

Image on the MCRs at t₀=0 hours.

Incubate the plate at 37° C. for 1 hours.

Image MCR1 at time point of 2 hours for bacterial and 4 hours for fungal species.

Calculate the areas of the microcolonies by analyzing the images and calculate their corresponding diameters, D, through the relation D=2*sqrt(area/3.1416). Then calculate the average diameter by averaging over all microcolonies.

Remove the microbial content of the MCR by a swab and resuspend it in 200 μL of TSB growth media (cell resuspension).

Serially dilute the cell resuspension in TSB with multiples of 10, and label the resulting samples as S10⁰, S10⁻¹, S10⁻², S10⁻³, and S10⁻⁴.

Plate the samples and incubate them for overnight.

Repeat steps 5 to 9 for MCR2 and MCR3, respectively at 3, 4, and optionally 6 hours for bacterial species (5, and 6 hours for fungal species). Count the overnight colonies and tabulate them.

Accordingly, calculate the number of microbial cells on the respective MCR.

Determine the growth rate by calculating the slope of the cell number versus time plot on a logarithmic-linear plot.

Plot average colony diameter versus the colony cell content.

Determine the average diameter at which the cell number in a microcolony reaches 10³ and 10⁵.

### Example 15: Measuring Recovery Rate of Separating Microbial Cell from Blood Sample and Colony Formation on Agar Plate

Recovery rates were measured for spiked whole blood samples as follows:

In a cartridge containing 4 mL of BLR of example 4, four mL of spiked whole blood sample (prepared according to example 2).

The blood sample and BLR were mixed 5 times.

The cartridge was centrifuged at 3000 g for 8 minutes.

A supernatant of 7.9 ml was moved to a waste chamber of the cartridge.

The first wash cycle was performed, by adding 2.9 mL of wash buffer to the residue, mixing the solution.

Centrifuge at 3000 g for 3 min, A supernatant of 2.9 ml was moved to the waste chamber.

Repeat steps 5 to 7 for second wash.

Remove the 100 μL residue (cell suspension)

Plate the cell suspension on an agar plate and incubate over night at 37° C.

Count the colonies and calculate the recovery with respect to the expected number according to control plate.

### Example 16: Experimental Staining Method

The microscopic imaging of the mixture was performed according to Wright's staining as follows:
1. Prepare a smear (film) of the mixture on a microscopic slide and allow to air dry.
2. Place the air-dried slide on the slide staining rack, smear-side facing upwards.
3. Cover the smear with undiluted staining solution to fix and partially stain the smear.
4. Wait 2 minutes.
5. Add approximately equal amount of water.
6. Wait for 5 minutes.
7. Rinse the stained smear.
8. Allow the slide to dry at room temperature and examine under 100× oil immersion microscope.

### Example 17: Effect of Calcium Concentration on Performance of Saponin-Based Blood Lysis Reagent

To illustrate the effects of high calcium content in the saponin material, two solutions were prepared in 8.5 mL of water, each using 96 mg of material having the same 86% w/w triterpenic saponin content by weight, as well as approximately the same polyphenol content (0.7 to 0.8% w/w) and polysaccharide content (3.8 to 4.0% w/w). However, one solution used material that had 0.6% w/w calcium, while the other had 0.3% w/w calcium. The solution with the higher amount of calcium appeared turbid while the other solution with the lower amount of calcium appeared clear and transparent.

Upon centrifugation at 4000 RPM for 3 minutes, the particles causing the turbidity in the high calcium solution were collected as a dark pellet at the bottom of the centrifuge tube, whereas no visible pellet was observed in the low calcium solution. Subsequent manual washings with 1 mM phosphate buffer and centrifugations were performed to give 100 to 120 μL of solutions from each solution, one which was dark and opaque as a result of the calcium turbidity, the other which was clear and transparent. This test illustrates how the turbidity caused by the calcium in the saponin material can be transferred to the final cell suspension when used in a lytic reagent for treating whole blood.

To test the tolerance of calcium in the lytic reagent coming from its saponin component, various batches of saponin solutions, with varying amounts of calcium, were mixed to prepare a set of saponin stocks with increasing concentrations of calcium. Each reagent contained 96 mg of saponin material which was composed of at least 85% w/w triterpenic saponins, between 0.7 and 0.8% w/w of polyphenols, and between 3.8 to 4.0% w/w polysaccharides, but with 0.33, 0.46, 0.64, and 0.79% w/w calcium by dry weight. These saponin solutions were used to make different lytic solutions each containing 0.20% Tergitol™ L-62, 6.25 mM MBCD, 4.25% SPS, and 5.00% Saponin (of a given stock) in 150 mM Acetate Buffer. 2 mL of each resulting blood lysis reagent had respectively 0.32, 0.43, 0.57, and 0.68 mg of Ca. These were filled in individual tubes. The higher concentrations of calcium led to greater solution turbidity over time in the reagents.

The tubes were used as blood collection devices. In each tube 7 mL of whole blood was drawn after which the tube was inverted 10 times to mix blood and the lytic reagent. Then 6.5 mL of each mixture was removed from the device and combined with 2 mL of 0.15M tris base activator in a centrifuge tube. After standard centrifugation and washing with 1 mM phosphate buffer, the resulting 100-120 μL of recovered cell suspension was spread over an agar plate and after 30 minutes was observed under a microscope.

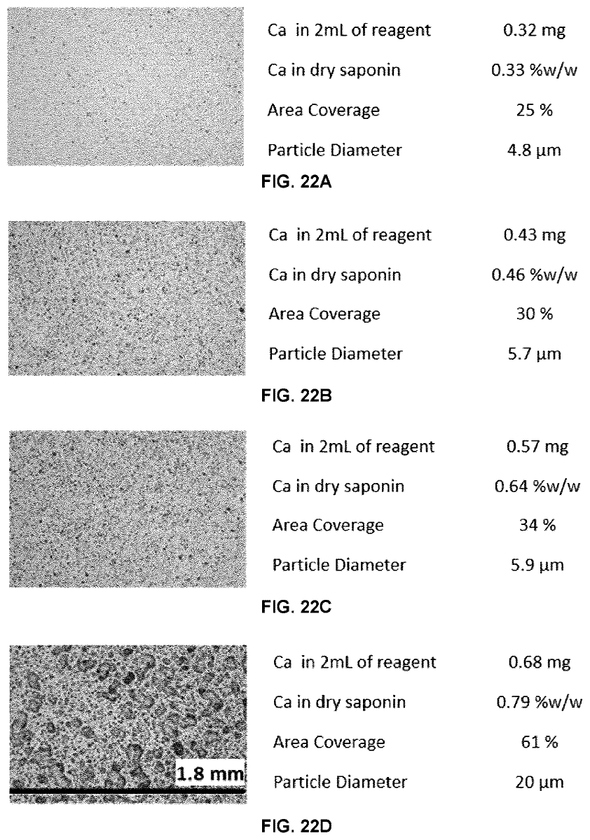

The images in FIGS. 22A-22D show typical sections of each plate. Corresponding to each frame, an indication is provided of the measured average debris particle size and the surface coverage (the percentage of plate area obstructed by debris). As it is observed, both average debris particle sizes and surface obstruction percentage increase with increasing amount of saponin Ca content. Without being bound to theory, it is speculated that the particles formed by the interaction of Ca and blood components are sedimenting during centrifugal washing and end up in the final cell suspension.

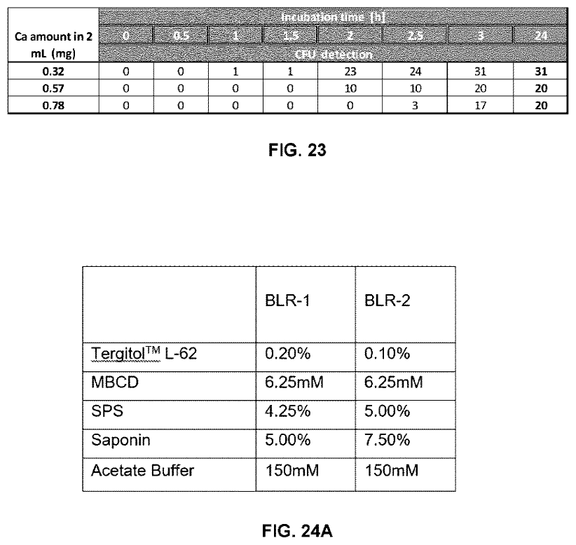

As it is observed from FIGS. 22A-22D, the lower the amount of calcium in the lytic reagent corresponds to lower number of sedimenting debris and lower debris size. This translates into improved microcolony detection and recovery. In one experiment, collected blood samples were spiked, in the different tubes (differing amounts of Ca as described above) with nominally 40 CFU of E. coli cells. 6.5 mL of each collected blood was processed following the standard method described in Examples and spread on agar gels. The gel surface was monitored while incubating. The Number of detected microcolonies are presented in FIG. 23. As it is observed, the reagents with lower Ca contents had better recovery and early detection times (Time to positivity; TP). According to this experiment the preferred upper limit of calcium content in 2 mL of lytic reagent is less than 0.78 mg. It is understood that if lower volumes of blood I (for instance 3 mL) is processed the amount of tolerable Ca may be 100 mg in 2 mL of the lytic reagent.

### Example 18: Beneficial Role of Methyl-Beta-Cyclodextrin in Acidic Blood Lysis Reagent

To determine the concentrations of MBCD that would prevent precipitation in a lytic reagent, various amounts were added to solutions containing 4.25% w/w of SPS, 5.00% w/w of saponin material, 0.20% w/w Tergitol™ L-62 and 0.15M sodium acetate. The final concentrations of MBCD were 0 mM, 3 mM, 6.25 mM, 9 mM and 15 mM. Five milliliters of each solution were then stored at either room temperature or at 45° C. After one week at 45° C., without the MBCD there was a white fluffy solid suspended in the solution whereas with 3 mM MBCD the amount of this solid was visibly reduced. No solid was observed in solutions with 6.25 mM and greater concentrations of MBCD. Similar results were observed in the set of solutions stored at room temperature, except the first observation of the solid in both the 0 mM and 3 mM MBCD samples were made after one month. Thus, the preferred amount of MBCD needed to stabilize a mixture of 4.25% w/w of SPS and 5.00% w/w of saponin would be at least over 3 mM in the lytic reagent filled in the collection tube. On the other hand, it has been observed that final concentrations of MBCD above 9 mM impacts the viability of some bacterial cells. leads to reduced cell recoveries in the final microbial cell suspension.

### Example 19: Example Method of Centrifugal Separation of Microbial Cells

The basified mixture was mixed for 1 minute using a vortex shaker. The mixture was then centrifuged for 8 minutes at 4000 RPM. The top supernatant was removed by pipette leaving 100 to 120 μL of cell suspension at the bottom of the conical tube. The cells were then re-suspended in 2.2 mL of 1 mM phosphate buffer by pipetting the suspension up and down, and then washed by mixing the suspension for 1 minute on a vortex shaker and centrifuged for 3 minutes at 4000 RPM. The supernatant was removed leaving 100 to 120 μL of cell suspension. This washing sequence was repeated two more times using fresh 2.2 mL 1 mM phosphate buffer each time to give a final 100 to 120 μL of clear cell suspension.

### Example 20: Direct Molecular Detection of Microbial Cells Separated from Whole Blood

The present example describes experiments that were performed to illustrate the capability of blood lysis reagents and processing methods employed according to the methods disclosed in the present disclosure to separate microbial cells from whole blood and employ the separated microbial cells for direct identification by molecular amplification. FIG. 24A is a table showing the composition of example blood lysis reagents employed in present experimental demonstrations of cell recovery and molecular amplification.

Whole blood, which had been collected into BD Vacutainer® SPS tubes, was employed to demonstrate the recovery of organisms from whole blood. The SPS tubes was comprised of about 8 ml of liquid including 80% whole blood and 20% SPS solution, so the total amount of whole blood in the sample was about 6.4 ml. After combining the 2 ml of the blood lysis reagent (BLR-1 shown in FIG. 24A) with 6 ml of SPS-whole blood samples to obtain a first mixture, the first mixture was spiked with approximately 50 CFU of microbial cells from various microbial species. The first mixtures were then left for a time delay of approximately a half hour at room temperature prior to addition of the alkaline buffer (as per the method of FIG. 19).

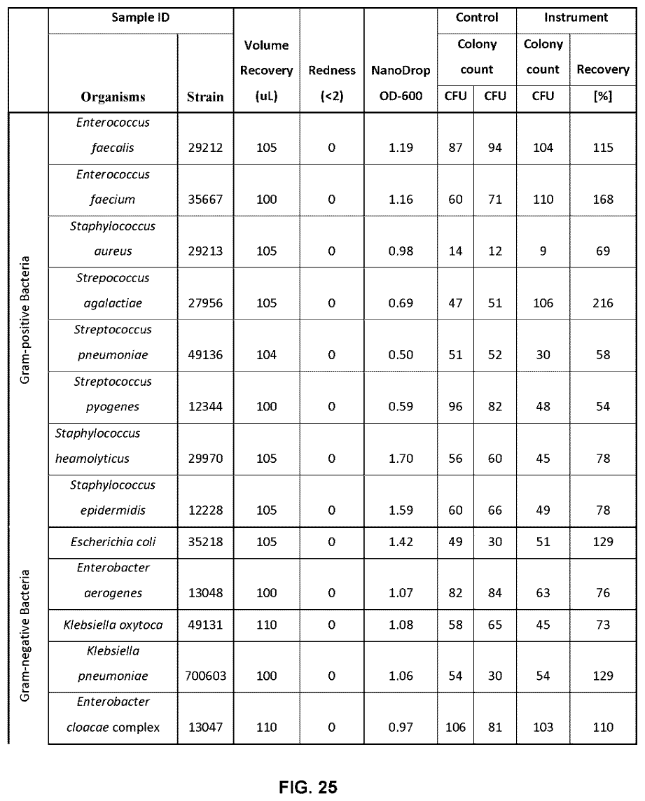

After the time half hour time delay, 75 μL of 3.0 M sodium carbonate was added to elevate the mixture's pH to approximately 8.3. The automated fluidic and centrifugal apparatus as described in International Patent Application No. PCT/CA2015/050449 was employed to perform centrifugal separation and washing of the microbial cells, and resuspension of the microbial cells within a 1 mM phosphate buffer, to give about 100 μL of a microbial suspension. Each resulting of the resulting microbial suspensions were applied onto a blood agar plate, incubated overnight at 37° C. and then counted for colonies. The results are shown in FIG. 25 for different microorganisms, showing a recovery in excess of 50% across all microbial species tested.

Experiments were also performed to demonstrate the ability of the blood lysis reagent and microbial cell separation method to provide a concentration and purified microbial cell suspension suitable for nucleic acid amplification, despite the risks posed by larger volume of whole blood in terms of the increased risk of incomplete hemolysis as well as the increased risk of introducing higher concentrations RT-LAMP inhibitors in the microbial cell suspension.

To test for the completeness of blood lysis and possible RT-LAMP inhibition, 8 mL of sample of SPS-whole blood, previously collected in a BD Vacutainer® SPS tube, was combined with 2 mL of either blood lysing reagent BLR-1 or BLR-2 in separate tubes to obtain a first mixture. The total volume of whole blood used from the SPS tube in the blood/lysing reagent mixtures was approximately 6.4 mL assuming the SPS tube is comprised of about 20% SPS solution. The tubes were capped and the mixtures of blood and blood lysing reagent were inverted 7 to 10 times to ensure proper mixing and hemolysis of the blood.

After one hour, 8 mL of the first mixture was mixed with 100 μL of 3.0 M sodium carbonate to obtain a second mixture, and to raise the pH above 9 to complete the lysis and make the second mixture less viscous. The 8 mL of second mixture is based on approximately 5.12 mL of lysed whole blood, 1.6 mL of the blood lysing reagent and 1.28 mL from the original SPS/saline solution from the BD Vacutainer® SPS tube. The cartridge was then placed into an automated fluidic and centrifugal apparatus as described in International Patent Application No. PCT/CA2015/050449 where it underwent automated centrifugation and washing with 1 mM phosphate buffer, to give approximately 120 μL of a third mixture, which was then subjected to heat lysing conditions at 95° C. for 10 minutes. It was then spiked with lysate derived from Klebsiella pneumoniae to give a fourth mixture with a final concentration of lysate of about 250 CFU/mL of cells. Then 4.38 μL of the fourth mixture was combined with a 0.63 μL solution of Klebsiella pneumoniae specific RT-LAMP primers and SYTO™ 9 dye to give a fifth 5 μL mixture composed of 87.5% of the third mixture from the automated process. This fifth mixture of lysate, dye, and primers was then used to rehydrate RT-LAMP enzymes dried at the bottom of an individual well of a PCR plate. As a control, the same lysate was spiked into 1 mM phosphate buffer (PB) and amplified using the same RT-LAMP protocol.

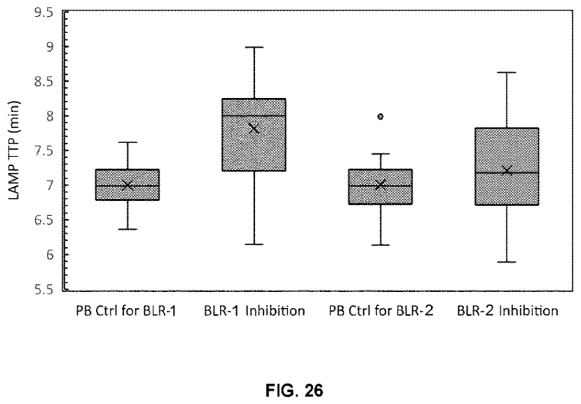

FIG. 26 summarizes the data of all RT-LAMP times-to-positivity's for K. pneumoniae lysate in blood matrices made from a total of 20 different blood donors using either BLR-1 or BLR-2. Each blood donor was tested with either BLR-1 or BLR-2 along with a control. With the third mixture derived from BLR-1 the mean TTP for the Klebsiella pneumoniae lysate was 7.82±0.77 minutes compared to its control at 7.00±0.36 minutes or a difference of 0.82 minutes. The maximum time difference that was measured from the control for BLR-1 was up to +2.0 minutes, while the minimum was −0.84 minutes. From BLR-2 the mean was 7.21±0.76 minutes while its control was 7.00±0.39 minutes or a difference of 0.21±0.52 minutes. The maximum time difference from the control for BLR-2 was +0.91 minutes, while the minimum was −1.10 min. Overall, BLR-2 gave a lower TTP (p=0.0162) and closer to its control (p=0.0029) than BLR-1 suggesting the presence of fewer RT-LAMP inhibitors in matrix.

Studies were also performed to demonstrate the automated separation of microbial cells from a whole blood sample and the rapid detection of the microbial cells via nucleic acid amplification and detection. Whole samples were spiked individually with either Gram-positive or Gram-negative species were processed according to the method of FIG. 19 to yield respective microbial cell suspensions. Each microbial cell suspension was then analyzed using an RT-LAMP assay using target specific primers.

For each test, 8 mL of whole human blood, previously collected in a BD Vacutainer® SPS tube was combined in a tube with 2 mL of either blood lysing reagent BLR-1or BLR-2. The tube was capped and the mixture of blood and lysing reagent was inverted 7 to 10 times to ensure proper mixing and hemolysis of the blood. 8 mL of the hemolyzed blood was removed and then spiked with approximately 30 to 50 CFU of a microorganism.

After 30 min to 1 hour the mixture was mixed, using an automated process within a fluidic cartridge, with 100 μL of 3.0 M sodium carbonate to raise the pH slightly greater than 9 to complete the lysis and make the mixture less viscous. In addition, 1.6 μL of SE-15 antifoam was added to ensure any foam generated in the cartridge's fluidic system would dissipate rapidly. The cartridge was processed by an automated fluidic and centrifugal apparatus as described in International Patent Application No. PCT/CA2015/050449, where it underwent automated centrifugation and washing with 1 mM phosphate buffer and centrifuging with 1 mM phosphate buffer, to give approximately 100 μL of microbial cell suspension.

The cells in the microbial cell suspensions were then lysed either by heat or using glass beads to release their mRNA for molecular amplification by RT-LAMP. One microliter of the lysate was then combined with a 4 μL mixture containing the RT-LAMP reagents, stabilizer, STYO™ 9 dye and a set of target specific primers. Between 4 to 6 reactions of 5 μL volume were amplified for each lysate in a LightCycler® 96 system and the average times-to-positivity were determined. As a control, the same 50 CFU of cells were added to 100 μL of 1 mM phosphate buffer, lysed either by heat or glass beads, and then amplified using the same RT-LAMP protocol.

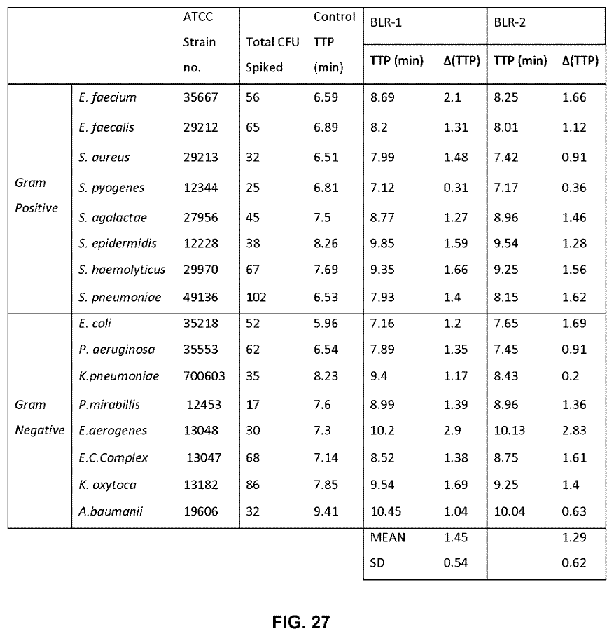

FIG. 27 shows the LAMP time to positivity for different microorganisms that were spiked into the blood. For BLR-1 the average time difference from the control (A(TTP)) was +1.36±0.64 min for all microorganisms tested. However, from the LAMP inhibition study described above, an average of 0.82 minutes of the time delay with BLR-1 can be attributed to molecular inhibition of the LAMP reaction by the blood matrix. For BLR-2 the time difference was +1.21±0.78 minutes, but an average of 0.21 minutes maybe attributable to molecular inhibition. It should be noted that the high activation pH to near 9 was beneficial to complete the digestion of the blood and to reduce its viscosity in order to produce a clear microbial cell suspension without any red colored debris. Although it is believed that this high PH level is detrimental to the viability of the cells, for a molecular assay some loss in viability appears to have less impact on the detection on microbial RNA.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1-32. (canceled)

33. A method of processing a whole blood sample suspected of containing microbial cells, the method comprising:
collecting the whole blood sample and mixing the whole blood sample with a blood lysis reagent to obtain a first mixture, the blood lysis reagent comprising saponin and sodium polyanethole sulfonate;
contacting the first mixture with an alkaline buffer and obtaining a second mixture with a viscosity that is less than a viscosity of the first mixture; and
separating microbial cells from the second mixture to obtain a suspension comprising the microbial cells.

34. The method according to claim 33 wherein the first mixture is contacted with the second mixture after a time delay between 15 minutes and 24 hours to permit partial digestion of blood debris within the first mixture prior to contact with the alkaline buffer.

35. (canceled)

36. (canceled)

37. The method according to claim 33 wherein the first mixture is contacted with the second mixture after a time delay between 15 minutes and 1 hour to permit partial digestion of blood debris within the first mixture prior to contact with the alkaline buffer.

38. (canceled)

39. (canceled)

40. The method according to claim 34, wherein a volume of the whole blood sample is between 5 ml and 10 ml.

41. The method according to claim 33, further comprising:
lysing microbial cells within the suspension to obtain a lysate; and
performing nucleic acid amplification to amplify at least one nucleic acid residing within the lysate, thereby obtaining an amplification product.

42. The method according to claim 41 further comprising detecting a presence of the amplification product.

43. The method according to claim 41 wherein at least one nucleic acid amplified by nucleic acid amplification comprises an antimicrobial resistance gene.

44-51. (canceled)

52. The method according to claim 41 wherein the nucleic acid amplification is performed in absence of performing nucleic acid extraction from the lysate.

53. The method according to claim 41 further comprising performing sequencing on the amplification product.

54. The method according to claim 33 further comprising:
inoculating the suspension of microbial cells onto a solid growth medium; and
monitoring the solid growth medium for a presence of a microcolony.

55. (canceled)

56. The method according to claim 54 wherein a composition of the blood lysis reagent and the time delay between forming the first mixture and contacting the first mixture with the alkaline buffer are selected such that after inoculating the suspension of microbial cells onto the solid growth medium, residual blood debris from the suspension partially covers an inoculated surface of the solid growth medium with a spatial areal fraction of less than 50 percent.

57-59. (canceled)

60. The method according to claim 54 wherein a composition of the blood lysis reagent and the time delay between forming the first mixture and contacting the first mixture with the alkaline buffer are selected such that a mean size of residual blood debris particles residing within the suspension is less than 10 micrometers.

61. (canceled)

62. The method according to claim 33 wherein the microbial cells are separated from the second mixture within one hour of contacting the first mixture with the alkaline buffer.

63. The method according to claim 33 wherein the blood lysis reagent is provided such that, after mixing the blood lysis reagent with the whole blood sample, the first mixture comprises a concentration of saponin between 0.75 and 60 mg/ml and concentration of sodium polyanethole sulfonate between 0.35 and 50 mg/ml.

64. The method according to claim 33 wherein a pH of the blood lysis reagent, prior to contact with the whole blood sample, resides between 3.5 and 8.

65. The method according to claim 33 wherein a concentration of calcium ions in the blood lysis reagent, relative to dry weight of saponin, is less than 0.5% w/w.

66. The method according to claim 33 wherein a concentration of calcium ions in the blood lysis reagent, relative to dry weight of saponin, is between 0.1% w/w and 0.5% w/w.

67. The method according to claim 33 wherein a pH of the alkaline buffer is between 8 to 9.

68. The method according to claim 33 wherein the blood lysis reagent further comprises cyclodextrin having a concentration between 0.1 mM and 20 mM.

69. (canceled)

70. The method according to claim 33 wherein the whole blood sample is collected and mixed with the blood lysis reagent at a collection site, and wherein the first mixture is received at a processing location that is remote from the collection site prior to contacting the first mixture with the alkaline buffer.

71-86. (canceled)
